(19) United States
(12) Patent Application Publication
(10) Pub. No.: US 2012/0027807 A1
(43) Pub. Date: Feb. 2, 2012

(54) TISSUE ENGINEERED MYOCARDIUM AND METHODS OF PRODUCTION AND USES THEREOF

(75) Inventors: Kenneth R. Chien, Cambridge, MA (US); Ibrahim J. Domian, Somerville, MA (US); Murali Chiravuri, Bridgeport, CT (US); Peter Van Der Meer, Groningen (NL); Kevin Kit Parker, Waltham, MA (US); Adam W. Feinberg, Pittsburgh, PA (US)

(73) Assignee: THE GENERAL HOSPITAL CORPORATION, Boston, MA (US)

(21) Appl. No.: 13/121,279

(22) PCT Filed: Oct. 9, 2009
(86) PCT No.: PCT/US2009/060224

§ 371 Date: Oct. 19, 2011

Related U.S. Application Data
(60) Provisional application No. 61/104,128, filed on Oct. 9, 2008.
(60) Provisional application No. 61/246,181, filed on Sep. 28, 2009.

(51) Int. Cl. A61K 35/34; A61K 9/00; C12Q 1/02; A61P 9/00; C12N 5/0775; C12N 5/10

(52) U.S. Cl. 424/400; 435/325; 435/366; 435/29; 424/93.7; 424/93.21

(57) ABSTRACT

The present invention generally relates to a population of committed ventricular progenitor (CVP) cells and their use to generate a tissue engineered myocardium, in particular two dimensional tissue engineered myocardium which is comparable to functional ventricular heart muscle. One embodiment of present invention provides a composition and methods for the production of a tissue engineered myocardium which has functional properties of cardiac muscle, such as contractibility (e.g. contraction force) and numerous properties of mature fully functional ventricular heart muscle tissue. In particular, in one embodiment, a composition comprising the tissue engineered myocardium comprises committed ventricular progenitor (CVP) cells seeded on a free-standing biopolymer structure to form functional ventricular myocardium tissue.

### CROSS REFERENCE TO RELATED APPLICATIONS

This application claims benefit under 35 U.S.C. 119(e) of U.S. Provisional Patent Application Ser. No. 61/104,128 filed on Oct. 9, 2008, and U.S. Provisional Patent Application 61/246,181 filed on Sep. 28, 2009, the contents of each are incorporated herein in their entity by reference.

### GOVERNMENT SUPPORT

This invention was made with Government support under Grant No: T32 HL002807 and HL079126 awarded by the National Institutes of Health (NIH). The Government has certain rights in the invention.

### FIELD OF THE INVENTION

The present invention generally relates to the field of tissue engineering, in particular, a tissue engineered composition comprising a scaffold and muscle tissue, such as cardiac muscle and/or myocardium, and methods for the production and use thereof.

### BACKGROUND OF THE INVENTION

Advanced heart failure is a major, unmet clinical need, arising from a loss of viable and/or fully functional cardiac muscle cells (37). Accordingly, designing new approaches to augment the number of functioning human cardiac muscle cells in the failing heart forms a foundation for modern regenerative cardiovascular medicine. Currently, a number of scientific studies and clinical trials have been designed to augment the number of functioning cardiac muscle cells via the transplantation of a diverse group of stem cells and progenitor cells outside of the heart, which might convert to functioning muscle and/or secondarily improve the function to cardiac muscle in the failing heart. However, while there have been encouraging early suggestions of a small therapeutic benefit, there has not been evidence for the robust regeneration of heart muscle tissue in these clinical studies (38, 39) thereby underscoring the need for new approaches.

One of the central challenges for cardiac cell based therapy has been the identification of an optimal cell type to drive robust cardiac myogenesis in cell-based therapy approaches. The ideal heart progenitor cell would have the properties of being isolated in sufficient quantities to drive clinically relevant levels of cardiac myogenesis, and also have the ability for renewal. In addition, it would be critical for the cell type of interest to be driven into a cardiomyogenic fate, as opposed to other closely related cardiovascular lineages, such as smooth muscle or conduction system muscle cells, that might carry electrophysiological side effects following their implantation.

Progenitor cells are marked by their ability for self-renewal and differentiation into various cardiac cell types. The progenitor cell is characterized by early-commitment from a pluripotent stem cell into a multipotent progenitor cell with the ability to differentiate into a unique subset of cell types. The advantage of using progenitor cells over standard embryonic stem (ES) cells is the pre-commitment towards a specific organ or tissue lineage, maximizing the differentiation rate into the cell-type of interest and prevention of teratomas formation.

Promising applications of these progenitor cells include regeneration of damaged tissue and its use in drug screening to assess functionality and potential toxicity. Previously cells from immortalized cell lines or primary tissues were used, however these had clear limitations in reproducibility and genetic abnormalities. Furthermore the use of these single cells in drug screening and regeneration is hampered by the inability to form tissue.

The properties that can be engineered depend on the cell/tissue/organ involved. It is a fact that a variety of the environmental factors and material properties are need to be controlled in concert. Further, the relative importance, magnitude and specificity of these elements that will direct differentiation are unique to each type of progenitor cell and also unique to the differentiated cell type sought for multipotent progenitors.

Progenitor cells can sometimes be used in cell-based therapies. In the case of treatment of cardiovascular conditions, existing methods are limited by several factors including viability of the progenitor cells and the ability of the progenitor cells to develop effectively into the desired cell phenotype, such as cardiac muscle, and/or to develop into functional tissue. Uncovering the pathways of developing functional cardiac tissue is a central question in cardiogenesis and has direct implications for cardiovascular regenerative medicine. In this regard, the inability to direct the differentiation of multipotent progenitors specifically to mature ventricular muscle remains a major obstacle for optimal in vivo cardiac myogenesis during cardiac repair following injury. Furthermore, while methods of cell based therapy using cells on scaffolds exist, their use is of limited benefit by their ability to support growth, differentiation and function of cells for a functional engineered cardiac tissue.

### SUMMARY

The present invention generally relates to a tissue engineered myocardium, in particular tissue engineered myocardium which is comparable to functional ventricular heart muscle. In particular, the present invention provides a composition and method of its production, of an improved tissue engineered myocardium that overcomes the limitations of existing tissue engineered myocardium, in that the tissue engineered myocardium of the present invention has functional properties of cardiac muscle, such as contractibility (e.g. contraction force) and has the properties of mature fully functional ventricular heart muscle tissue.

As disclosed herein, the inventors have discovered a method to produce a functional tissue engineered myocardium by seeding scaffolds or structures with a population of committed ventricular progenitor (CVP) cells to form functional tissue engineered myocardium and cardiac tissue which is capable of contracting. Accordingly, the inventors have discovered a method to produce tissue engineered cardiac tissue which will result in vastly superior cardiac muscle function as compared to existing tissue engineered cardiac tissue.

One aspect of the invention relates to a composition comprising a substantially pure population of committed ventricular progenitor (CVP) cells. Committed ventricular progenitor (CVP) are a subpopulation of second heart field (SHF) progenitors and are uniquely committed to the right ventricle (RV) and outflow tract (OFT). Thus, the inventors have discovered that CVP cells differentiate into ventricular myocytes. The inventors discovered CVP cells using a combination of a two color reporter system and fluorescently activated cell sorting (FACS) to identify and isolate discrete populations of cardiac progenitor cells which represent different sub-populations of first heart field (FHF) and second heart field (SHF) progenitors. In particular, the inventors identified and isolated three distinct unique populations of cardiac progenitors: (1) double labeled dsRed+/eGFP+(R+G+) population representing second heart field (SHF) progenitors which are committed to the right ventricle (RV) and outflow tract (OFT) progenitors, and herein is referred to as a committed ventricular progenitor (CVP), (2) single labeled dsRed+ negative (referred to herein as dsRed +/eGFP− or R+G−) population representing a different subpopulation of second heart field (SHF) progenitors which are committed to primitive isl1+ pharyngeal mesoderm (PM) progenitors, and (3) a single labeled eGFP+ (referred to herein as dsRed −/eGFP+ or R−G+) population representing first heart field (FHF) progenitors which are committed to the left ventricle (LV) and inflow tract progenitors. Accordingly, one aspect of the present invention relates to a population of CVP cells, or a substantially pure population of CVP cells, where a CVP cell are positive for at least two markers selected from the group of Mef2c, Nkx2.5, Tbx20, Isl1, miR-208, miR-143, miR-133a, miR-133b. In some embodiments, a CVP cell are positive for at least two, or at least 3, or at least 4, or at least 5 or at least 7 or at least 8 markers selected from the group of Mef2c, Nkx2.5, Tbx20, Isl1, miR-208, miR-143, miR-133a, miR-133b. In some embodiments, a CVP cell can express additional markers, such as at least 1, or at least two, or at least 3, or at least 4, or at least 5 or at least 7 or at least 8 or at least 9 or more markers selected from the group consisting of; GATA4, GATA6; Tropinin T, Troponin C, BMP7, BMP4, BMP2, miR-1, miR-143, miR-689. Furthermore, in combination with at least two or more of the above-listed positive expression markers, a CVP cell can be identified by their lack of, or low level expression of the following negative markers; the primary heart field marker Tbx5, and other markers, such as Snai2, miR-200a, miR-200b, miR-199a, miR-199b, miR-126-3p, miR-322, CD31.

Another aspect of the present invention relates to a composition comprising the tissue engineered myocardium, also referred to muscle thin film (MTF) as disclosed herein, comprising a scaffold and a substantially pure population of committed ventricular progenitor (CVP) cells, wherein a committed ventricular progenitor cell is a secondary heart field (SHF) progenitor which is capable of giving rise to mature ventricular cardiomyocytes. Accordingly, a substantially pure population of committed ventricular progenitors (CVPs) on an appropriate scaffold can result in a mature strip of fully functional cardiac muscle tissue, herein referred to a muscle thin film (MTF). The mature strip of fully functional cardiac muscle tissue as disclosed herein is capable of generating a force comparable to neonatal cardiomyocytes. As disclosed herein, the thin biological film seeded with a patterned layer of CVPs generates a fully functional ventricular muscle tissue that has the ability to generate force, tension and contractility that is quantitatively similar to biological thin films constructed from neonatal ventricular muscle tissue (16).

In one aspect of the invention, the tissue engineered myocardium in the form of muscular thin film (MTF), in which a population of committed ventricular progenitor (CVP) are plated on a scaffold, such a thin film of polydimethylsiloxane elastomer to create a muscular thin film (MTF) as described in Feinberg et al (2007) and disclosed in International Patent Application WO2008/045506, which is incorporated herein in its entirety by reference. The inventors have demonstrated that the MTF as disclosed herein can beat spontaneously at ˜20 beats/minute and that it can be paced by a field stimulator such that it could control the beats to a simulation, for example at 0.5-1.0 Hz to produce force as generated with biological thin films constructed from neonatal ventricular muscle tissue.

Another aspect of the present invention relates to methods of production of the tissue engineered myocardium disclosed herein, comprising coating a scaffold, such as a thin film of polydimethylsiloxane elastomer scaffold with a population of CVPs, where the CVPs are seeded onto the thin polydimethylsiloxane elastomer film in a particular pattern. In some embodiments, the pattern has been engineered on the substrate to create anisotropic uni-axial alignment of the seeded CVP cells, as discussed in further detail below.

Another aspect of the present invention relates to uses of the tissue engineered myocardium disclosed herein, for example, its use in assays to identify agents which affect (e.g. increase or decrease) the contractile force and/or contractibility of the tissue engineered myocardium in the presence of the agent as compared to a control agent or absence of an agent. Such an assay is useful to identify an agent which has a cardiotoxic effect, such as an agent which decreases contractile force, and/or cardiomyocyte atrophy, and/or results in another dysregulation of contractibility, such as arrhythmia or abnormal contraction rate. In another embodiment, such an assay is useful to identify an agent which has a cardiotoxic effects by increasing contractile force and/or other types of dysregulation such as an increase in contraction rate and could lead to the development of cardiac muscle hypertrophy.

In another embodiment, the tissue engineered myocardium disclosed herein can be used to study a cardiovascular disease. By way of an example only, the tissue engineered myocardium can comprise genetically modified cardiomyogenic progenitors, for example cardiomyogenic progenitors carrying a mutation, polymorphism or other variant of a gene (e.g. increased or decreased expression of a heterologous gene) which can be assessed to see the effects of such a gene variant on the contractile force and contractible ability of the tissue engineered myocardium. Such a tissue engineered myocardium comprising genetically modified cardiomyogenic progenitors can also be used to identify an agent which attenuates (e.g. decreases) any dysfunction in contractibility or contraction force as a result of the genetically modified cardiomyogenic progenitors, or alternatively can be used to identify an agent which augments (e.g. increases) any dysfunction in contractibility or contraction force as a result of the genetically modified cardiomyogenic progenitors.

In another embodiment, the tissue engineered myocardium as disclosed herein can be used for prophylactic and therapeutic treatment of a cardiovascular condition or disease. By way of an example only, in such an embodiment, a tissue engineered myocardium as disclosed herein can be administered to a subject, such as a human subject by way of transplantation, where the subject is in need of such treatment, for example, the subject has, or has an increased risk of developing a cardiovascular condition or disorder.

The compositions comprising the tissue engineered myocardium as disclosed herein are distinguished from other engineered cardiac tissue by virtue of the cells on the scaffold (e.g. the identity of the myocardial committed progenitors) present on the scaffold. The cardiomyogenic progenitor cells, such as the ventricular myogenic progenitor cells of the engineered cardiac tissue can be identified by cell specific markers. The identity of a cardiomyogenic progenitor cell can be detected by reacting with an agent which specifically binds to a protein and/or nucleic acid of such a marker expressed by the cardiomyogenic progenitor cell. Detection is accomplished using standard techniques such as electron, fluorescent and/or atomic force microscopy, as well as fluorescent cell sorting (FACS) and other cell sorting methodologies.

### BRIEF DESCRIPTION OF THE DRAWINGS

FIG. 1 shows a the generation of SHF-dsRed/Nkx2.5-eGFP double transgenic mouse embryonic stem cell lines. FIG. 2A shows a schematic flow diagram of the strategy to generate the SHF-dsRed/Nkx2.5-eGFP double transgenic mouse embryonic stem cell lines; SHF-dsRed mice were interbred with Nkx2.5-eGFP, ED3.5 blastocysts were isolated and cultured on irradiated mouse embryonic fibroblasts in the presence of Leukemia Inhibitory Factor (LIF) to generate double transgenic ESC.

FIGS. 2A-2D show characterization of cardiac progenitors isolated form developing double transgenic mouse embryos (ED9.5) mouse embryos. Double transgenic mouse embryos (ED9.5) were trypsinized into single cell suspension and were FACS sorted. FIGS. 2A-2C show flow cytometry images of the three populations of cells were isolated; FIG. 2A shows the dsRed+/eGFP+(R+G+) cells representing the RV and the outflow tract, FIG. 2B shows the R+G− cells representing the pharyngeal mesoderm, and FIG. 2C shows R−G+ cells representing the LV and the inflow tract.

FIGS. 3A-3E show genome wide transcriptional profiling of ESC derived and embryonic cardiac progenitors. FIG. 3A shows a representative flow cytometry plot of double-labeled SHF-dsRed/Nkx2.5-eGFP ESC lines which were differentiated by hanging droplet formation and were dissociated into single cell suspension on EB day 6. FACS sorting revealed 4 populations of cells, double negative (NEG), dsRed+/eGFP+(R+G+), dsRed+ single positive (R+G−), and eGFP+ single positive (R−G+). FIG. 4B shows a tree-structured dendrogram to demonstrate hierarchical clustering of gene expression and revealed distinct patterns of gene expression of known and novel cardiac markers. The gene expression analysis was performed on total RNA from FACS sorted cardiac progenitors was isolated and arrayed on the Affymetrix 430.20 chip. FIG. 3C shows 4 populations of cells obtained from ED9.5 double transgenic embryos dissociated into single cell suspension. FACS sorting revealed 4 populations of cells, double negative, dsRed+/eGFP+(R+G+) representing the RV and OFT, dsRed+(R+G−) representing the PM, and eGFP+ (R−G+) representing the LV and inflow tract. A representative flow cytometry plot is shown. FIG. 3D shows quantitative PCR analysis on RNA isolated from embryonic progenitors confirmed a distinct pattern of gene expression. FIG. 3E shows a table of primers used for qRT-PCR analysis.

FIGS. 4A-4B show genome wide profiling of miRNA in cardiac progenitor populations. FIG. 4A shows a tree-structured dendrogram to demonstrate hierarchical clustering of gene expression from total RNA from R+G+, R+G−, R+G+, and R−G− (negative) cells was arrayed using a miRCURY™ LNA Array (v.9.2). One-way hierarchical clustering of miRNAs and progenitor populations revealed distinct patterns of miRNA expression in the different cardiac progenitor populations. FIG. 4B shows results from quantitative PCR analysis on total RNA isolated from embryonic progenitors confirmed a distinct pattern of miRNA expression of known and novel cardiac specific miRNA.

FIG. 5 is a schematic representation of the process used to tissue engineer myocardium from ES cell derived cardiac progenitors. Step 1, ES cells are cultured using standard methods to allow population doublings and then grown into embryoid bodies where the ES cells enter a progenitor state. Step 2, the embryoid bodies are digested into a single-cell suspension and a FACS system is used to isolate the progenitor cell populations based on the fluorescent reporter system genetically engineered into the cells. Step 3, the progenitor cell population of interest is seeded onto a scaffold or surface engineered to direct differentiation be controlling cell-cell, cell-surface and cell-medium interactions. Step 4, progenitor cells differentiate into a neo-myocardium at which point they may be used as grown or harvested for other cell-based applications.

FIGS. 6A-6C show examples of flow cytometry images of purified myogenic cardiac progenitors isolated from embryonic stem cells differentiating in vitro. Embryoid bodies were allowed to differentiate in vitro for 6 days. FIGS. 6A-6C show the results of the isolation of positive GFP/dsRed (R+G+) from total EBs which were dissociated into single cell and isolated by Flow Cytometry. FIG. 6D shows an example photomicrograph image of immunostaining the purified committed ventricular progenitor cells (CVP) (positive GFP/dsRed, R+G+) plated on micropatterned tissue engineered surfaces, which results in the formation of organized myocardial fibrils. The committed ventricular progenitor (CVP) cells (positive GFP/dsRed, R+G+) plated on micropatterned tissue engineered surfaces were immunostained for nuclei, SM-MHC and sarcomeric a-actinin.

FIG. 7 shows quantitative RT-PCR for Troponin T (TnT) 5 days after culturing. Ds-Red/GFP (R+G+) labeled cells show the highest TnT content.

FIGS. 8A-8C show the cell cycle analysis with Hoechst DNA staining. FIG. 8A shows the cell cycle analysis of total ES cells at EB day 6. FIG. 8B shows the cell cycle analysis of dsRed+/eGFP+ (R+G+) cells at EB day 6, and FIG. 8C shows the cell cycle analysis of dsRed+/eGFP+(R+G+) cells after 5 days of culturing, showing that most committed ventricular progenitor are differentiated and become senescent.

FIGS. 9A-9F show functional engineered tissue derived from Nkx2.5-eGFP/SHF-dsRed myocardial progenitor cells. FIG. 9A shows dsRed+/eGFP (R+G+), dsRed+(R+G−), eGFP+(R−G+), and R−G− (negative) cells which were FACS sorted from double transgenic ED 9.5 embryos and plated on micro-patterned substrate consisting of alternating layers of fibronectin and pluronics and allowed to differentiate an additional 6-7 days to generate a muscular thin film (MTF, as described in herein in the methods section of the Examples). Alpha actinin and smMHC staining revealed that R+G+ progenitors gave rise to 95% (+/−1.6%) cardiac myocytes (CM) and 4% (+/−1%) smooth muscle (SM). R−G+ progenitors gave rise to 67% (+/−9%) cardiac myocytes and 17% (+/−6%) smooth muscle. R+G− progenitors gave rise to 38% (+/−20%) CM and 10% (+/−4%) SM. R−G− (negative) cells gave rise to 6% (+/−2%) cardiac myocytes and 15% (+/−2%) smooth muscle. Representative fluorescence microscopy images of smMHC and sarcomeric α-actinin immunostaining is shown. To generate ES derived ventricular myocyte strips, double transgenic ESC were differentiated in vitro and R+G+ progenitors were FACS sorted on EB day 6 and plated on the MTF. FIG. 9B shows fluorescence microscopy images of ES derived ventricular myocyte strips which demonstrates the linear arrangement of mature ventricular cardiomyocytes with clearly visible striations. Representative fluorescence microscopy images for immunostaining for smMHC and sarcomeric α-actinin is shown. FIG. 9C shows an image of a representative profile of a spontaneous action potentials recorded from ES derived MTF. R+G+ derived cardiomyocytes revealed a typical ventricular profile in 11 of 12 consecutive cells. FIG. 9D shows a bar graph demonstrating the replicative capacity of cells the R+G+ derived cardiomyocytes, as determined by hoechst staining and FACS analysis were used to perform cell cycle analysis of undifferentiated ESC, EB day 6 cardiac progenitors, and their differentiated progeny. Both undifferentiated ESC and EB day 6 cardiac progenitors had a high replicative capacity (40-60% of cells in S/G2 phase) but the differentiated progeny had a low replicative capacity (<10% S/G2 phase). FIG. 9E shows R+G+ES derived cardiac progenitors which were plated on a thin film of polydimethylsiloxane elastomer to create a muscular thin film (MTF) as described herein and also described in Feinberg et al (2007) (16). Field stimulation was used to induce cyclical contraction of the MTF that result in MTF bending. A typical contraction from the end of diastole to peak systole and back to diastole lasts ˜500 ms. FIG. 9E also shows the amount of bending of the MTF due to cyclical contraction at 0 ms, 120 ms, 240 ms, 360 ms and 480 ms. FIG. 9E shows that the MTF bending can be used to calculate the contractile force generated by ES-derived the tissue engineered myocardium as described herein in the Materials and Methods section of the Examples. In this example, the peak systolic stress generated is ˜13 kPa at 0.5 Hz pacing, comparable to the peak systolic stress generated by MTFs engineered from neo-natal mouse ventricular cardiomyocytes. MTF bending was used to calculate the contractile force generated by the ES derived myocardial tissue. A peak systolic stress generated is ˜13 kPa at 0.5 Hz pacing, comparable to the peak systolic stress generated by MTFs engineered from neo-natal ventricular cardiomyocytes (Feinberg et al (2007) (16).

FIGS. 10A-10D show the tissue engineered myocardium on a muscular thin film derived from cardiac progenitor cells. FIG. 10A shows an image of the MTF when heptanol washed IN to block gap junctions, and FIG. 10B shows the contractile force over time after heptanol was washed in. FIG. 10 shows an image of the same MTF when heptanol is washed out to remove the blocking of the gap junctions, and FIG. 10D shows the contractile force over time after heptanol was washed out, showing partial washout restores contractions. Coupling of cardiomyocytes by gap junctions (e.g. Connexin 43) was reversibly blocked by the wash-in and then wash-out of heptanol. All the heptanol was not washed-out resulting in the reduced contractility after cell-cell electrical coupling was restored.

FIG. 11 shows a schematic diagram demonstrating the identification of a fully committed ventricular cardiac progenitor cell in the Islet-1 lineage that is capable of limited differentiation into ventricular cardiomyocytes or ventricular myocardium.

FIG. 12 shows a schematic diagram of a consensus clustering of biological triplicates of total RNA was arrayed on the Affymetrix 430.20 chip. Microarray expression profiling on RNA isolated from 3 distinct populations of cardiac progenitors. The double transgenic ES cell line was allowed to differentiate in vitro and FACS sorting was performed on EB day 6. 1,000,000 cells were isolated from each of the 4 populations of cells. Experiments were repeated in biological triplicates for a total of 12 microarrays. Total RNA was arrayed on the Affymetrix 430.20 chip. The labeling, hybridization, and scanning of the microarray experiments were performed at the Dana Farber Cancer Institute Microarray Core Facility. Data analysis was performed on the GenePattern software package. Consensus clustering was performed using a hierarchical clustering algorithm (kₘₐₓ=5). This revealed that the genome wide transcriptional profile of each of the 4 populations of cells clustered together in replicate experiments, validating the experimental reproducibility.

FIGS. 13A-13C show representative image profiles of spontaneous action potentials from anisotropic ESC-derived tissue. FIG. 13A shows a representative action potential of tissue derived from dsRed+progenitors which demonstrate an action potential immature phenotype. FIG. 13B shows a representative action potential of tissue derived from eGFP+progenitors which demonstrates a triggered ventricular phenotype. FIG. 13C shows a representative action potential of tissue derived from dsRed+/eGFP+progenitors, which demonstrates a mature ventricular phenotype.

FIGS. 14A-14B show the level of expression of cardiac transcription factors and structural proteins during in vitro differentiation. The double transgenic ES cell line was allowed to differentiate in vitro and FACS sorting was performed on EB day 6. dsRed+/eGFP+(R+G+), dsRed+(R−G+), and eGFP+(R−G+) cardiac progenitor were isolated and plated on MTF. Cells were then allowed to differentiate for an additional 2 days (EB d6+2) or an additional 5 days (EB d6+5). Undifferentiated ESC, EB day 6 cardiac progenitors, and their differentiated progeny) were harvested and RNA was isolated and assayed for expression of isl1 or Tbx5 was performed by real-time PCR analysis. FIG. 14A shows that in the eGFP+/dsRed+(R+G+) population and the dsRed+(R+G−) population, Isl1 is expressed at peak levels on EB day 6 and this decreases with further expansion and differentiation such that it is turned off completely by day 10 of differentiation. FIG. 14B shows that in the eGFP+(R−G+) population, Tbx5 is expressed at peak levels on EB day 6 and this decreases with further expansion and differentiation.

FIG. 15 shows gene expression analysis of isolated cardiac progenitors. DAPI and Ki67 staining was performed on ESC derived cardiac progenitors which were cultured 5 days, to quantify total cell number and proportion of cycling cells (Ki67+cells/total cells) in R+G+, R−G+ and R+G−populations. SD shown (n=4).

FIG. 16A-16B shows differentiation potential of cardiac progenitors. Embryonic and ESC-derived cardiac progenitors were cultured on fibronectin coated slides (fibronectin) or micro-patterns for five days. FIG. 16A shows the results from cell counting analysis to quantify the relative number of cardiomyocytes (CM) (sarcomeric-actinin positive) or smooth muscle (SM) (smMHC positive) derived from embryonic progenitors. FIG. 16B shows the results from cell counting analysis to quantify the relative number of CM (sarcomeric_-actinin positive) or SM (smMHC positive) derived from ESC progenitors. R+G+populations resulted in the most CM (p<0.001). No significant differences were observed in SM differentiation (p=0.38−1.0). P-values for the differences in CM differentiation are displayed.

FIGS. 17A-17B show Engineered ventricular tissue from R+G+progenitors. FIG. 17A shows R+G+(n=12), R+G−(n=5), and R−G+(n=5) progenitors were allowed to differentiate and single cell patch clamp recordings were performed. AP morphology was assessed for typical four-phase ventricular action potential. FIG. 17B shows a representative spontaneous AP from R+G+ derived cardiomyocytes.

FIGS. 18A-18B show FACS re-analysis of purified progenitor populations. FIG. 18A shows double-labeled SHF-dsRed/Nkx2.5-eGFP ESC lines were differentiated by hanging droplet formation and were dissociated into single cell suspension on EB day 6. FACS sorting was performed to isolate 4 populations of cells: R+G+, R+G−, R−G+, and R−G− cells. FACS reanalysis was then performed to determine the purity of sorted cells. FIG. 18B shows FACS analysis of ED9.5 double transgenic embryos, which were dissociated into single cell suspension. R+G+, R+G−, R−G+, and R−G− cells were FACS purified as with the ESC. FACS reanalysis was performed to determine the purity of sorted cells. Representative FACS plots are shown.

FIGS. 19A-19B show a comparison of transcriptional profile of embryonic vs. ESC-derived cardiac progenitors. ED9.5 double transgenic embryos were dissociated into single cell suspension. FACS sorting was performed to isolate 4 populations of cells as follows: R+G+, R+G−, R−G+, and R−G−. qPCR analysis was performed on 100 structural and regulatory genes that were overexpressed in cardiac progenitor populations. 50 of the structural and regulatory genes are shown in FIG. 19A, and 50 of the structural and regulatory genes which were analyzed are shown in FIG. 19A. All values were normalized against the R−G− population, defined as “1”. Hierarchical clustering was then performed with the Hierarchical Clustering Module of GenePattern (M. Buckingham, S. Meilhac, S. Zaffran, Nat Rev Genet 6, 826 (November 2005), which is incorporated herein in its entirety by reference) with un-centered correlation and pairwise complete-linkage of log2 transformed expression levels. A tree-structured dendrogram was then generated and revealed distinct patterns of gene expression in embryonic and ESC derived progenitors. Red color represents an expression level above the mean and blue color represents expression lower than the mean. Overall, most genes that were over-expressed in the ESC derived progenitors were also overexpressed in embryonic progenitors; the patterns were not identical, however. These differences are likely due to the differences between ESC in vitro differentiation and true embryonic development. Nonetheless, the ESC based system does allow for the purification of a far greater number of progenitor cells from a renewable cell source. The ESC based system is therefore ideally suited for applications that require a large number of cells such as tissue engineering applications.

FIG. 20 shows marker expression during in vitro determination of cardiac progenitors. Cardiac progenitors were isolated from EB day6 and cultured for an additional five days. Total RNA was isolated on EB day6, after two days of additional culture (EB day 6+2), and after 5 days of additional culture (EB day 6+5). Expression of Isl1 (R+G+ and R+G−) or Tbx5 (R−G+) as well as Troponin T (all populations) was assayed by qPCR.

FIGS. 21A-21B shows patch-clamp analysis of the differentiated progeny of ESC derived cardiac progenitors. The double transgenic ESC line was allowed to differentiate in vitro and FACS sorting was performed on EB day 6. R+G+, R+G−, and R−G+ cardiac progenitors were isolated and plated on a micro-patterned surface. Cells were allowed to differentiate for an additional 5 days and patch clamp analysis was performed as described in the supplementary materials and methods. Two representative action potentials of contracting cells derived from R+G+ (FIG. 21A), R+G− (FIG. 21B) and R−G+ (FIG. 21C) progenitors are shown. The R+G+ population gave rise to a homogenous population with ventricular like four phase action potentials. The R+G− population gave rise to immature appearing action potentials. The R-G+ population gave rise to a heterogeneous population that included both types of morphologies.

FIG. 22 shows the effect of Tetrododoxin (TTX) on the transmembrane action potential of R+G+ derived ventricular myocytes. Double transgenic ESC line was allowed to differentiate in vitro and R+G+progenitors were FACS isolated on EB day 6. Progenitors were then allowed to differentiate for an additional 5 days and patch clamp analysis was performed as above. Single cell patch clamp was performed and tetrodotoxin (TTX), a potent sodium channel inhibitor (S. Martin-Puig, Z. Wang, K. R. Chien, Cell Stem Cell 2, 320 (2008), which is incorporated herein in its entirety by reference), was applied by constant perfusion catheter to the patched cells (arrow head). After 60 seconds of perfusion the TTX was washed off (open arrow). TTX ablated the action potential, consistent with the sodium dependency of ventricular action potentials. Experiments were repeated on four individual cells, with the same result. A representative sample is shown. The ablation of AP was observed at the single cell level in the culture conditions described below and this may vary according with culture conditions.

FIG. 23 shows the radius of Curvature of ESC-derived MTF. The radius of curvature plot is plotted as a function of time to demonstrate the bending of the MTF that occurs during 0.5 Hz paced contractions at the tissue scale. ESC derived 2-dimensional myocardial tissue contracted synchronously. The change in radius of curvature is inversely proportional to cardiomyocyte stress generation along the longitudinal axis and was calculated using a modified Stoney's equation as described in (E. Dodou, S. M. Xu, B. L. Black, Mech Dev 120, 1021 (September 2003), which is incorporated herein in its entirety by reference). The stress generated by progenitor derived cardiac tissue at peak systole was measured at ˜5 kPa.

FIG. 24 shows a table of the normalized (log2 transformed) ratios of miRNA expression level in cardiac progenitors. Total RNA from R+G+, R+, G+, and R−G− cells was arrayed on a miRCURY™ LNA Array (v.9.2). The relative expression level of progenitor samples was normalized against a pooled control sample. The log2 of the ratio is shown as the median of replicated measurements of the miRNA. “NA” in the row containing an miRNA indicates that 2 or more of the 4 replicated measures of this miRNA were below the background detected by the image analysis software. miR199 was only detectable in the R+G− population and this resulted in an inability to calculate a SD and exclusion from the heat map in FIG. 19A and 19B.

FIG. 25 shows a table of the statistical analysis of embryonic progenitor mRNA profile. P-values are reported for multiple comparisons of mRNA expression profiles between the different embryonic progenitor cells. The inventors used Bonferroni post-hoc testing to correct for multiple comparisons.

FIG. 26 shows a table of statistical analysis of embryonic progenitor miRNA profile. P-values are reported for multiple comparisons of miRNA expression profiles between the different embryonic progenitor cells. The inventors Bonferroni post-hoc testing to correct for multiple comparisons.

FIG. 27 shows a table S4 of the action potential (AP) properties of ES derived cells. FACs sorted R+G+, R+G−, and R−G+progenitors were plated and allowed to differentiate on micro-patterned surfaces and then subjected to patch clamp analysis as described in Figure S8. Values are represented as means+/−SD. Observations are the average of 5 to 6 recordings for each cell population. AMP, amplitude; APD 50 and APD 90, action potential durations at 50 and 90% depolarization respectively; Vmax, maximum upstroke velocity. The mean Vmax and APD durations were lower for the immature R+G− population compared to the ventricular-like R+G+ cells. The indices of the R−G+ cells showed more variability than the R+G+ subset, reflective of more heterogeneity than the cells of the R+G+ population, which showed a more uniform ventricular-like AP morphology (see also FIG. 20).

### DETAILED DESCRIPTION

As disclosed herein, the inventors have discovered a method to produce a functional tissue engineered myocardium by seeding scaffolds or structures with a population of committed ventricular progenitor (CVP) cells. Accordingly, the inventors have discovered a method to produce functional tissue engineered myocardium a which is capable of contracting and has vastly superior cardiac muscle function as compared to existing tissue engineered cardiac tissue.

One aspect of the present invention relates to a composition of a tissue engineered myocardium comprising a substantially pure population of committed ventricular progenitors (CVPs) on an appropriate scaffold to generate a mature strip of fully functional cardiac muscle tissue, herein referred to a muscular thin film (MTF). The substantially pure population of committed ventricular progenitor (CVP) cells used to generate the tissues engineered myocardium is a population of secondary heart field (SHF) progenitors, and are capable of giving rise to mature ventricular cardiomyocytes.

One aspect of the present invention relates to the use of the CVPs in combination with engineered substrates and scaffolds for controlled differentiation of the CVPs into mature ventricular cardiomyocytes resulting in the generation of functional cardiac tissue.

One aspect of this invention relates to the discovery of methods to isolate the CVPs from other secondary heart field (SHF) progenitors for use in generating functional cardiac tissue such as the MTF tissue and tissue engineered myocardium as disclosed herein.

In some embodiments, the scaffold used to generate the MTF tissue as disclosed herein is patterned, for example the scaffold is engineered so that the cellular environment at multiple spatial scales (nanometer to meter) is modified in order to direct progenitor cells down specific differentiation pathways and to subsequently organize the CVP cells into two-dimensional (2D) and three-dimensional (3D) myocardial tissue structures.

In some embodiments, the inventors demonstrate by using a population of ES-derived committed ventricular progenitor (CVP) cells, the methodology to differentiate the CVPs into mature ventricular cardiomyocytes and the formation of engineered cardiac muscle, such as engineered myocardium. The inventors demonstrate that the functional performance of MTF tissue generated from ES-derived CVP cells is comparable to myocardial tissue constructed from neonatal cardiomyocytes.

This invention represents a key advancement in the strategy for engineering functional myocardium from an embryonic stem (ES) cell source. This technology is based on two key capabilities. First advance is the ability to maintain ES cells where the cells can proliferate indefinitely while maintaining their pluripotency. Then, allowing the ES cells to differentiate in vitro and isolating sub-populations of the differentiated cells that express specific markers for cardiac progenitor cells. These cardiac subpopulations have a restricted tri-potency destined to form differentiated cells (cardiomyocyte, endothelial, and smooth muscle cells) for cardiac tissue overcoming issues of teratoma formation. The second advance is the integration of these progenitor cells into an engineered scaffold or substrate where the environmental cues have been controlled to direct differentiation. The cellular environment is engineered from the nanometer to micrometer to millimeter to macroscopic length cells. Factors that are engineered include but are not limited to material mechanical properties, material solubility, spatial patterning of bioactive compounds, spatial patterning of topological features, soluble bioactive compounds, mechanical perturbation (cyclical or static strain, stress, shear, etc . . . ), electrical stimulation, and thermal perturbation.

In concert, these two advancements allow a multipotent progenitor cell population to be isolated from ES cells and driven towards a differentiated cell type at a high-efficiency that surpasses all current methodologies. Further, experimental results demonstrate unequivocally that the differentiated myocardium derived from ES derived CVPs have functional properties (contractile force) comparable to myocardium from neonatal cardiomyocytes. Accordingly, any CVP which is derived from an ES cell or other source, such as induced pluripotent stem (IPS) cells, or the reprogramming of somatic cells can be used in the present invention to generate tissue engineered myocardium and MTF tissue as disclosed herein. Accordingly, the present invention provides the capability to generate functional myocardium from a renewable cell source. In some embodiments, use of a population of CVPs are ES derived or derived by some from some other renewable cell source, such as from reprogrammed cells such as iPS cells, enables the MTF to be generated from patient-specific CVPs populations. Such patient-specific MTF are valuable in the use of the MTF for advanced assays for drug screening, as well as for therapeutic purposes such as regeneration and prognostication of disease states.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “cardiomyocyte” as used herein broadly refers to a muscle cell of the heart. The term cardiomyocyte includes smooth muscle cells of the heart, as well as cardiac muscle cells, which include also include striated muscle cells, as well as spontaneous beating muscle cells of the heart.

The term “first heart field linage” and “FHF lineage” are used interchangeably herein and refers to cell which is capable of giving rise to progeny that differentiate into cardiac tissue in the anatomically located primitive left ventricle (LV) and inflow tract.

The terms “first heart field progenitor” and “primary heart field progenitor” are used interchangeably herein and refers to a progenitor cell which typically is Is11 negative and give rise to cardiac tissue of the left ventricle (LV) and inflow tract (IT).

The terms “second heart field linage” and “anterior heart field linage” or “SHF linage” are used interchangeably herein and refers to a cell, such as progenitor cell, which are capable (without dedifferentiating or reprogramming) of giving rise to progeny that includes a variety of cardiac tissues, including cardiomyocytes, smooth muscle cells, pacemaker and conduction systems and endothelial cells. Progenitors which belong to the secondary heart field linage are typically multipotent Isll+ multipotent progenitors which co-express Nkx2.5 and can undergo self-renewal.

The terms “second heart field progenitor” and “anterior heart field progenitor” and “SHF progenitor” are used interchangeably herein and refer to a progenitor cell of the second heart field, or anterior heart field, and is typically a multipotent Isll+ multipotent progenitor which co-expresses Nkx2.5 and can undergo self-renewal and is also capable (without dedifferentiating or reprogramming) of giving rise to progeny that include cardiomyocytes, smooth muscle cells, pacemaker and conduction systems and endothelial cells. Secondary heart field progenitors can be subdivided into categories, (i) a secondary heart field progenitor subtype which gives rise to pharyngeal mesoderm (PM) tissue and are characterized by positive expression for markers Mef2c, IsL1+, Snai2 and (ii) a secondary heart field progenitor subtype, herein termed a “committed ventricular progenitor” which gives rise to the right ventricle (RV) and outflow tract (OFT) as discussed herein.

The term “ventricular myogenic progenitor” is used interchangeably herein with the term “Committed Ventricular Progenitor” or “CVP” as used herein, refers to a progenitor cell which is capable of proliferation and giving rise to more progenitor cells having the ability to generate a large number of mother cells that can in turn give rise to differentiated, or differentiable daughter cells which can eventually terminally differentiate primarily into ventricular cardiomyocytes. In particular, CVPs are a subset of secondary heart field (SHF) progenitors and are capable (without dedifferentiating or reprogramming) of giving rise to right ventricle (RV) and outflow tract (OFT) progenitors and differentiating into cardiomyocytes, in particular ventricular cardiomyocytes to give rise to ventricular cardiac muscle. A CVP cell is capable of expanding in culture and assemble into fully mature, rod shaped ventricular cardiac muscle cells. Stated another way, a CVP cell is capable of differentiating into a ventricular cardiomyocyte and giving rise to cardiac tissue in the anatomically located primitive right ventricle (RV) and outflow tract (OFT). For example, a substantially pure population of CVPs will give rise to approximately about at least 75% . . . , or at least about 80% . . . , or at least about 85% . . . , or at least about 90% . . . , or at least about 95% or higher than 95% population of ventricular cardiomyocytes, or any percentage integer between 75% and 100% population of ventricular cardiomyocytes. A population of CVP cells is therefore capable of generating ventricular cardiomyocytes which are capable of generating fully mature ventricular muscle tissue that has the ability to generate force, tension and contractibility similar to neonatal myocardium or neonatal myocardiocytes. As used herein the CVP cells that are ventricular myogenically committed progenitor cells can be identified by being positive for at least one or at least two of the following markers selected from the group comprising; developmentally regulated cardiogenic transcription factors; Mef2c, Nkx2.5, Tbx20, IsL1+, GATA4, and GATA6; myocardial markers Tropinin T, Troponin C, BMP signalling molecules; BMP7, BMP4, BMP2 and miRNA molecules; miR-208, miR-143, miR-133a, miR-133b, miR-1, miR-143, miR-689. Furthermore, in combination with at least two or more of the above-listed positive expression markers, the CVP cells can be identified by their lack of, or low level expression of the following negative markers; the primary heart field marker Tbx5, and other markers, such as Snai2, miR-200a, miR-200b, miR-199a, miR-199b, miR-126-3p, miR-322, CD31. A ventricular myogenic progenitor cell as referred to herein is also referred to as a ventricular myogenically committed progenitor cell.

The term “neonatal mouse ventricular cardiomyocytes” as used herein refers to a cardiomyocyte obtained from a mouse which is obtained from the second heart field of ventricular tissue.

The term “myogenically committed” or “myogenic committed” refers to a cell, such as a progenitor cell, such as a ventricular myogenic progenitor cell, which differentiated into a substantially pure population of cardiac muscle cells such as cardiomyocytes.

The term “cardiomyocyte” refers to a muscle cell of the heart (e.g. a cardiac muscle cell). A cardiomyocyte will generally express on its cell surface and/or in the cytoplasm one or more cardiac-specific marker. Suitable cardiomyocyte-specific markers include, but are not limited to, cardiac troponin I, cardiac troponin-C, tropomyosin, caveolin-3, GATA-4, myosin heavy chain, myosin light chain-2a, myosin light chain-2v, ryanodine receptor, and atrial natriuretic factor.

The terms “enriching” or “enriched” are used interchangeably herein and mean that the yield (fraction) of cells of one type is increased by at least 10% over the fraction of cells of that type in the starting culture or preparation.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a preparation of one or more partially and/or terminally differentiated cell types, refer to a population of cells that contain fewer than about 20%, more preferably fewer than about 15%, 10%, 8%, 7%, most preferably fewer than about 5%, 4%, 3%, 2%, 1%, or less than 1%, of cells that are not stem cells or stem cell progeny.

A “marker” as used herein describes the characteristics and/or phenotype of a cell. Markers can be used for selection of cells comprising characteristics of interest. Markers vary with specific cells. Markers are characteristics, whether morphological, functional or biochemical (enzymatic) characteristics particular to a cell type, or molecules expressed by the cell type. Preferably, such markers are proteins, and more preferably, possess an epitope for antibodies or other binding molecules available in the art. A marker may consist of any molecule found in, or on the surface of a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method commonly available to one of skill in the art.

A “reporter gene” as used herein encompasses any gene that is genetically introduced into a cell that adds to the phenotype of the stem cell. Reporter genes as disclosed in this invention are intended to encompass fluorescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny.

The term “engineered” with respect to myocardium or neo-myocardium as used herein refers to the artificial creation (or de novo generation) of myocardial tissue. In the instant disclosure, engineered myocardium refers to the artificial creation of myocardial tissue from components of CVP and an appropriate scaffold such as biopolymer scaffolds as disclosed herein. Without being limited to theory, tissue engineering is the use of a combination of cells, engineering and materials methods, and suitable biochemical and physio-chemical factors for the de novo generation of tissue or tissue structures. Such engineered tissue or tissue structures are useful for therapeutic purposes to improve or replace biological functions. Engineered tissue covers a broad range of applications, including but not limited to utility in the repair or replace portions of, or whole tissues (e.g., heart, cardiac tissue, ventricular myocardium and other tissues such as bone, cartilage, blood vessels, bladder, etc.) or assays for identifying agents which modify the function of parts of, or entire organs without the need to obtain such organs from a subject. Engineered tissue that is generated typically has desired certain mechanical and structural properties for proper functioning.

The term “tissue engineered myocardium” refers to the artificial creation of myocardial tissue from components such as CVP and an appropriate scaffold such as biopolymer scaffolds as disclosed herein.

The term “derived from” used in the context of a cell derived from another cell means that a cell has stemmed (e.g. changed from or produced by) a cell which is a different cell type. In some instances, for e.g. a cell derived from an iPS cell refers to a cell which has differentiated from an iPS cell. Alternatively, a cell can be converted from one cell type to a different cell type by a process referred to as transdifferention or direct reprogramming. Alternatively, in the terms of iPS cells, a cell (e.g. iPS cell) can be derived from a differentiated cell by a process referred to in the art as dedifferentiation or reprogramming.

The terms “muscular thin film” and “MTF” are used interchangeably herein and refer to a two-dimensional biopolymer scaffolds comprising CVP cells stacked to form a three-dimensional (3D) structure tissue engineered myocardial composition. The 2D biopolymer scaffold can be seed with CVP cells before or after the stacking to form a 3D structure. Typically, the MTF is used in methods for therapeutic use or for screening agents, as disclosed herein.

The term “biodegradable” as used herein denotes a composition that is not biologically harmful and can be chemically degraded or decomposed by natural effectors (e.g., weather, soil bacteria, plants, animals).

The term “bioresorbable” refers to the ability of a material to be reabsorbed over time in the body (e.g. in vivo) so that its original presence is no longer detected once it has been reabsorbed.

The term “bioreplaceable” as used herein, and when used in the context of an implant, refers to a process where de novo growth of the endogenous tissue replaces the implant material. A bioreplacable material as disclosed herein does not provoke an immune or inflammatory response from the subject and does not induce fibrosis. A bioreplaceable material is distinguished from bioresorbable material in that bioresorbable material is not replaced by de novo growth by endogenous tissue.

The terms “processed tissue matrix” and “processed tissue material” are used interchangeably herein, to refer to native, normally cellular tissue that as been procured from an animal source, for example a mammal, and mechanically cleaned of attendant tissues and chemically cleaned of cells and cellular debris, and rendered substantially free of non-collagenous extracellular matrix components. In some embodiments, the processed tissue matrix can further comprise non-cellular material naturally secreted by cells, such as intestinal submucosa cells, isolated in their native configuration with or without naturally associated cells.

As used herein the term “submucosal tissue” refers to natural extracellular matrices, known to be effective for tissue remodelling, that have been isolated in their native configuration. The submucosal tissue can be from any animal, for example a mammal, such as but not limited to, bovine or porcine submucosal tissue. In some embodiments, the submucosal tissue is derived from a human, such as the subject into which it is subsequently implanted (e.g. autograft transplantation) or from a different human donor (e.g. allograft transplantation). The submucosa tissue can be derived from intestinal tissue (autograft, allograft, and xenograft), stomach tissue (autograft, allograft, and xenograft), bladder tissue (autograft, allograft, and xenograft), alimentary tissue (autograft, allograft, and xenograft), respiratory tissue (autograft, allograft, and xenograft) and genital tissue (autograft, allograft, and xenograft), and derivatives of liver tissue (autograft, allograft, and xenograft), including for example liver basement membrane and also including, but not limited to, dermal extracellular matrices (autograft, allograft, and xenograft) from skin tissue.

The term “substantially” as used herein means a proportion of at least about 60%, or preferably at least about 70% or at least about 80%, or at least about 90%, at least about 95%, at least about 97% or at least about 99% or more, or any interger between 70% and 100%.

The term “cardiac progenitor cell” and “CPC” are used interchangeably herein refers to a progenitor cell which is capable of proliferation and giving rise to more progenitor cells having the ability to generate a large number of mother cells that can in turn give rise to differentiated, or differentiable daughter cells which can eventually terminally differentiate primarily into cells of the heart tissue, including endothelial lineages, muscle lineages (smooth, cardiac and skeletal muscles).

The term “contractibility” is used interchangeably herein with “cell contractility” and a refers to the force (or contraction force) generated by unified coordinated contraction a collection of cells, such as CVP cells or CVP-derived cells. The contractility of a plurality of cells is measured by biophysical and biomechanical properties of the force transmission.

The term “phenotype” refers to one or a number of total biological characteristics that define the cell or organism under a particular set of environmental conditions and factors, regardless of the actual genotype.

The term “contacting” or “contact” as used herein as in connection with contacting a CVP cell, either present on a support, or absence of a support, with an agent as disclosed herein, includes subjecting the cell to a culture media which comprises that agent. Where the differentiated cell is in vivo, contacting the differentiated cell with a compound includes administering the compound in a composition to a subject via an appropriate administration route such that the compound contacts the differentiated cell in vivo.

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. In some embodiments, a pluripotent cell is an undifferentiated cell.

The term “pluripotency” or a “pluripotent state” as used herein refers to a cell with the ability to differentiate into all three embryonic germ layers: endoderm (gut tissue), mesoderm (including blood, muscle, and vessels), and ectoderm (such as skin and nerve), and typically has the potential to divide in vitro for a long period of time, e.g., greater than one year or more than 30 passages.

The term “multipotent” when used in reference to a “multipotent cell” refers to a cell that is able to differentiate into some but not all of the cells derived from all three germ layers. Thus, a multipotent cell is a partially differentiated cell. Multipotent cells are well known in the art, and examples of muiltipotent cells include adult stem cells, such as for example, hematopoietic stem cells and neural stem cells. Multipotent means a stem cell may form many types of cells in a given lineage, but not cells of other lineages. For example, a multipotent blood stem cell can form the many different types of blood cells (red, white, platelets, etc . . . ), but it cannot form neurons.

The term “multipotency” refers to a cell with the degree of developmental versatility that is less than totipotent and pluripotent.

The term “totipotency” refers to a cell with the degree of differentiation describing a capacity to make all of the cells in the adult body as well as the extra-embryonic tissues including the placenta. The fertilized egg (zygote) is totipotent as are the early cleaved cells (blastomeres). As indicated above, there are different levels or classes of cells falling under the general definition of a “stem cell.” These are “totipotent,” “pluripotent” and “multipotent” stem cells. The term “totipotent” refers to a stem cell that can give rise to any tissue or cell type in the body. “Pluripotent” stem cells can give rise to any type of cell in the body except germ line cells. Stated another way, pluripotent refers to cells which can give rise to a mesoderm lineage, ectoderm lineage or endoderm lineage. iPS cells are pluripotent cells. Stem cells that can give rise to a smaller or limited number of different cell types are generally termed “multipotent.” Thus, totipotent cells differentiate into pluripotent cells that can give rise to most, but not all, of the tissues necessary for fetal development. Pluripotent cells undergo further differentiation into multipotent cells that are committed to give rise to cells that have a particular function. For example, multipotent hematopoietic stem cells give rise to the red blood cells, white blood cells and platelets in the blood.

As used herein, the term “somatic cell” refers to any cell other than a germ cell, a cell present in or obtained from a pre-implantation embryo, or a cell resulting from proliferation of such a cell in vitro. Stated another way, a somatic cell refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for reprogramming a differentiated cell can be performed both in vivo and in vitro (where in vivo is practiced when an differentiated cell is present within a subject, and where in vitro is practiced using isolated differentiated cell maintained in culture). In some embodiments, where a differentiated cell or population of differentiated cells are cultured in vitro, the differentiated cell can be cultured in an organotypic slice culture, such as described in, e.g., meneghel-Rozzo et al., (2004), Cell Tissue Res, 316(3);295-303, which is incorporated herein in its entirety by reference.

As used herein, the term “adult cell” refers to a cell found throughout the body after embryonic development.

As used herein, the terms “iPS cell” and “induced pluripotent stem cell” are used interchangeably and refers to a pluripotent cell artificially derived (e.g., induced by complete or partial reversal) from an undifferentiated cell (e.g. a non-pluripotent cell).

The term “progenitor cell” is used herein to refer to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art.

The term “embryonic stem cell” is used to refer to the pluripotent stem cells of the inner cell mass of the embryonic blastocyst (see U.S. Pat. Nos. 5,843,780, 6,200,806, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (e.g., sequences naturally found in the cells) but in a non-naturally occurring arrangement (e.g., a coding region linked to a promoter from a different gene), or altered versions of native sequences, etc. The process of transferring the nucleic into the cell is referred to as “transducing a cell” and can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell.

The term “isolated” or “enriching” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “enriching” is used synonymously with “isolating” cells, and means that the yield (fraction) of cells of one type is increased by at least 10% over the fraction of cells of that type in the starting culture or preparation.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from. In some embodiments, the isolated population is an isolated population of reprogrammed cells which is a substantially pure population of reprogrammed cells as compared to a heterogeneous population of cells comprising reprogrammed cells and cells from which the reprogrammed cells were derived.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of reprogrammed cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not reprogrammed cells or their progeny as defined by the terms herein. In some embodiments, the present invention encompasses methods to expand a population of reprogrammed cells, wherein the expanded population of reprogrammed cells is a substantially pure population of reprogrammed cells.

The terms “renewal” or “self-renewal” or “proliferation” are used interchangeably herein, and refers to a process of a cell making more copies of itself (e.g. duplication) of the cell. In some embodiments, reprogrammed cells are capable of renewal of themselves by dividing into the same undifferentiated cells (e.g. pluripotent or non-specialized cell type) over long periods, and/or many months to years. In some instances, proliferation refers to the expansion of reprogrammed cells by the repeated division of single cells into two identical daughter cells.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “lineages” as used herein refers to a term to describe cells with a common ancestry, for example cells that are derived from the same cardiovascular stem cell or other stem cell, or cells with a common developmental fate. By way of an example only, a cell that is of endoderm origin or is “endodermal linage” this means the cell was derived from an endodermal cell and can differentiate along the endodermal lineage restricted pathways, such as one or more developmental lineage pathways which give rise to definitive endoderm cells, which in turn can differentiate into liver cells, thymus, pancreas, lung and intestine.

The term “cell line” or “clonal cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time) and in some instances has the potential to propagate indefinitely. It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. A clonal cell line can be a stem cell line or be derived from a stem cell, and where the clonal cell line is used in the context of a clonal cell line comprising stem cells, the term refers to stem cells which have been cultured under in vitro conditions that allow proliferation without differentiation for months to years. Such clonal stem cell lines can have the potential to differentiate along several lineages of the cells from the original stem cell.

The term “modulate” is used consistently with its use in the art, e.g., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”,“increase” or “enhance” or “activate” are all used herein to generally mean an increase by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “progenitor cells” is used synonymously with “stem cell.” Generally, “progenitor cells” have a cellular phenotype that is more primitive (e.g., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate. It is possible that cells that begin as progenitor cells might proceed toward a differentiated phenotype, but then “reverse” and re-express the progenitor cell phenotype.

The term “reprogramming” as used herein refers to the transition of a differentiated cell to become a pluripotent progenitor cell. Stated another way, the term reprogramming refers to the transition of a differentiated cell to an earlier developmental phenotype or developmental stage. A “reprogrammed cell” is a cell that has reversed or retraced all, or part of its developmental differentiation pathway to become a progenitor cell. Thus, a differentiated cell (which can only produce daughter cells of a predetermined phenotype or cell linage) or a terminally differentiated cell (which can not divide) can be reprogrammed to an earlier developmental stage and become a progenitor cell, which can both self renew and give rise to differentiated or undifferentiated daughter cells. The daughter cells themselves can be induced to proliferate and produce progeny that subsequently differentiate into one or more mature cell types, while also retaining one or more cells with parental developmental potential. The term reprogramming is also commonly referred to as retrodifferentiation or dedifferentiation in the art. A “reprogrammed cell” is also sometimes referred to in the art as an “induced pluripotent stem” (iPS) cell.

In the context of cell ontogeny, the term “differentiated”, or “differentiating” is a relative term. A “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an atrial precursor), and then to an end-stage differentiated cell, such as atrial cardiomyocytes or smooth muscle cells which plays a characteristic role in a certain tissue type, and may or may not retain the capacity to proliferate further. The term “differentiated cell” is meant any primary cell that is not, in its native form, pluripotent as that term is defined herein. The term a “differentiated cell” also encompasses cells that are partially differentiated, such as multipotent cells, or cells that are stable non-pluripotent partially reprogrammed cells. In some embodiments, a differentiated cell is a cell that is a stable intermediate cell, such as a non-pluripotent partially reprogrammed cell. It should be noted that placing many primary cells in culture can lead to some loss of fully differentiated characteristics. Thus, simply culturing such cells are included in the term differentiated cells and does not render these cells non-differentated cells (e.g. undifferentiated cells) or pluripotent cells. The transition of a differentiated cell (including stable non-pluripotent partially reprogrammed cell intermediates) to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture. In some embodiments, the term “differentiated cell” also refers to a cell of a more specialized cell type derived from a cell of a less specialized cell type (e.g., from an undifferentiated cell or a reprogrammed cell) where the cell has undergone a cellular differentiation process.

The term “differentiation” as referred to herein refers to the process whereby a cell moves further down the developmental pathway and begins expressing markers and phenotypic characteristics known to be associated with a cell that are more specialized and closer to becoming terminally differentiated cells. The pathway along which cells progress from a less committed cell to a cell that is increasingly committed to a particular cell type, and eventually to a terminally differentiated cell is referred to as progressive differentiation or progressive commitment. Cell which are more specialized (e.g., have begun to progress along a path of progressive differentiation) but not yet terminally differentiated are referred to as partially differentiated. Differentiation is a developmental process whereby cells assume a more specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, in the context of this specification, the terms “differentiation” or “differentiated” refer to cells that are more specialized in their fate or function than at one time in their development. For example in the context of this application, a differentiated cell includes a ventricular cardiomyocyte which has differentiated from a CVP cell, where such CVP can in some instances be derived from the differentiation of an ES cell, or alternatively from the reprogramming of an induced pluripotent stem (iPS) cell, or in some embodiments from a human ES cell line. Thus, while such a ventricular cardiomyocyte cell is more specialized than the time in which it had the phenotype of a CVP, it can also be less specialized as compared to when the cell existed as a mature cell from which the iPS cell was derived (e.g. prior to the reprogramming of the cell to form the iPS cell).

The development of a cell from an uncommitted cell (for example, a stem cell), to a cell with an increasing degree of commitment to a particular differentiated cell type, and finally to a terminally differentiated cell is known as progressive differentiation or progressive commitment. A cell that is “differentiated” relative to a progenitor cell has one or more phenotypic differences relative to that progenitor cell. Phenotypic differences include, but are not limited to morphologic differences and differences in gene expression and biological activity, including not only the presence or absence of an expressed marker, but also differences in the amount of a marker and differences in the co-expression patterns of a set of markers.

As used herein, “proliferating” and “proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The terms “mesenchymal cell” or “mesenchyme” are used interchangeably herein and refer in some instances to the fusiform or stellate cells found between the ectoderm and endoderm of young embryos; most mesenchymal cells are derived from established mesodermal layers, but in the cephalic region they also develop from neural crest or neural tube ectoderm. Mesenchymal cells have a pluripotential capacity, particularly embryonic mesenchymal cells in the embryonic body, developing at different locations into any of the types of connective or supporting tissues, to smooth muscle, to vascular endothelium, and to blood cells.

The term “tissue” refers to a group or layer of similarly specialized cells which together perform certain special functions. The term “tissue-specific” refers to a source or defining characteristic of cells from a specific tissue.

The term “genetically modified” as used herein refers to a cell or entity, by human manipulation such as chemical, physical, viral or stress-induced or other means that has undergone mutation or selection; or that an exogenous nucleic acid has been introduced to the cell or entity through any standard means, such as transfection; such that the cell or entity gas acquired a new characteristic, phenotype, genotype, and/or gene expression product, including but not limited to a gene marker, a gene product, and/or a mRNA, to endow the original cell or entity, at a genetic level, with a function, characteristic, or genetic element not present in non-genetically modified, non-selected counterpart cells or entities.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied. The terms “peptide(s)”, “protein(s)” and “polypeptide(s)” are used interchangeably herein.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (e.g., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent). The term mutation is used interchangeably herein with polymorphism in this application.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

As used herein, the term “gene” or “recombinant gene” refers to a nucleic acid comprising an open reading frame encoding a polypeptide, including both exon and (optionally) intron sequences.

The term “recombinant,” as used herein, means that a protein is derived from a prokaryotic or eukaryotic expression system.

As used herein, the term “vector” refers to a nucleic acid molecule capable of transporting another nucleic acid to which it has been linked Preferred vectors are those capable of autonomous replication and/or expression of nucleic acids to which they are linked Vectors capable of directing the expression of genes to which they are operatively linked are referred to herein as “expression vectors”.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g EPV and EBV vectors.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “regulatory sequence” and “promoter” are used interchangeably herein, refers to a generic term used throughout the specification to refer to nucleic acid sequences, such as initiation signals, enhancers, and promoters, which induce or control transcription of protein coding sequences with which they are operatively linked In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, e.g., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

The terms “subject” and “individual” are used interchangeably herein, and refer to an animal, for example a human, to whom treatment, including prophylactic treatment, with methods and compositions described herein, is or are provided. For treatment of those infections, conditions or disease states which are specific for a specific animal such as a human subject, the term “subject” refers to that specific animal. The terms “non-human animals” and “non-human mammals” are used interchangeably herein, and include mammals such as rats, mice, rabbits, sheep, cats, dogs, cows, pigs, and non-human primates.

The term “regeneration” means regrowth of a cell population, organ or tissue after disease or trauma.

As used herein, the phrase “cardiovascular condition, disease or disorder” is intended to include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The term “disease” or “disorder” is used interchangeably herein, and refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, indisposition or affection.

The term “pathology” as used herein, refers to symptoms, for example, structural and functional changes in a cell, tissue or organs, which contribute to a disease or disorder. For example, the pathology may be associated with a particular nucleic acid sequence, or “pathological nucleic acid” which refers to a nucleic acid sequence that contributes, wholly or in part to the pathology, as an example, the pathological nucleic acid may be a nucleic acid sequence encoding a gene with a particular pathology causing or pathology-associated mutation or polymorphism. The pathology may be associated with the expression of a pathological protein or pathological polypeptide that contributes, wholly or in part to the pathology associated with a particular disease or disorder. In another embodiment, the pathology is for example, is associated with other factors, for example ischemia and the like.

As used herein, the terms “treat” or “treatment” or “treating” refers to both therapeutic treatment and prophylactic or preventative measures, wherein the object is to prevent or slow the development of the disease, such as slow down the development of a cardiac disorder, or reducing at least one adverse effect or symptom of a cardiovascular condition, disease or disorder, e.g., any disorder characterized by insufficient or undesired cardiac function. Adverse effects or symptoms of cardiac disorders are well-known in the art and include, but are not limited to, dyspnea, chest pain, palpitations, dizziness, syncope, edema, cyanosis, pallor, fatigue and death. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

The term “effective amount” as used herein refers to the amount of therapeutic agent of pharmaceutical composition to reduce at least one or more symptom(s) of the disease or disorder, and relates to a sufficient amount of pharmacological composition to provide the desired effect. The phrase “therapeutically effective amount” as used herein, e.g., of population of atrial progenitors or atrial myocytes as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to effect a therapeutically or prophylatically significant reduction in a symptom or clinical marker associated with a cardiac dysfunction or disorder when administered to a typical subject who has a cardiovascular condition, disease or disorder.

A therapeutically or prophylatically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, that the total daily usage of the compositions and formulations as disclosed herein will be decided by the attending physician within the scope of sound medical judgment. The exact amount required will vary depending on factors such as the type of disease being treated.

With reference to the treatment of a cardiovascular condition or disease in a subject, the term “therapeutically effective amount” refers to the amount that is safe and sufficient to prevent or delay the development or a cardiovascular disease or disorder. The amount can thus cure or cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, for example treatment of a rodent with acute myocardial infarction or ischemia-reperfusion injury, and any treatment or administration of the compositions or formulations that leads to a decrease of at least one symptom of the cardiovascular disease or disorder as disclosed herein, for example, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment. In embodiments where the compositions are used for the treatment of a cardiovascular disease or disorder, the efficacy of the composition can be judged using an experimental animal model of cardiovascular disease, e.g., animal models of ischemia-reperfusion injury (Headrick JP, Am J Physiol Heart circ Physiol 285;H1797;2003) and animal models acute myocardial infarction. (Yang Z, Am J Physiol Heart Circ. Physiol 282:H949:2002; Guo Y, J Mol Cell Cardiol 33;825-830, 2001). When using an experimental animal model, efficacy of treatment is evidenced when a reduction in a symptom of the cardiovascular disease or disorder, for example, a reduction in one or more symptom of dyspnea, chest pain, palpitations, dizziness, syncope, edema, cyanosis, pallor, fatigue and high blood pressure which occurs earlier in treated, versus untreated animals. By “earlier” is meant that a decrease, for example in the size of the tumor occurs at least 5% earlier, but preferably more, e.g., one day earlier, two days earlier, 3 days earlier, or more.

As used herein, the term “treating” when used in reference to a cancer treatment is used to refer to the reduction of a symptom and/or a biochemical marker of cancer, for example a reduction in at least one biochemical marker of cancer by at least about 10% would be considered an effective treatment. Examples of such biochemical markers of cardiovascular disease include a reduction of, for example, creatine phosphokinase (CPK), aspartate aminotransferase (AST), lactate dehydrogenase (LDH) in the blood, and/or a decrease in a symptom of cardiovascular disease and/or an improvement in blood flow and cardiac function as determined by someone of ordinary skill in the art as measured by electrocardiogram (ECG or EKG), or echocardiogram (heart ultrasound), Doppler ultrasound and nuclear medicine imaging. A reduction in a symptom of a cardiovascular disease by at least about 10% would also be considered effective treatment by the methods as disclosed herein. As alternative examples, a reduction in a symptom of cardiovascular disease, for example a reduction of at least one of the following; dyspnea, chest pain, palpitations, dizziness, syncope, edema, cyanosis etc. by at least about 10% or a cessation of such systems, or a reduction in the size one such symptom of a cardiovascular disease by at least about 10% would also be considered as affective treatments by the methods as disclosed herein. In some embodiments, it is preferred, but not required that the therapeutic agent actually eliminate the cardiovascular disease or disorder, rather just reduce a symptom to a manageable extent.

Subjects amenable to treatment by the methods as disclosed herein can be identified by any method to diagnose myocardial infarction (commonly referred to as a heart attack) commonly known by persons of ordinary skill in the art are amenable to treatment using the methods as disclosed herein, and such diagnostic methods include, for example but are not limited to; (i) blood tests to detect levels of creatine phosphokinase (CPK), aspartate aminotransferase (AST), lactate dehydrogenase (LDH) and other enzymes released during myocardial infarction; (ii) electrocardiogram (ECG or EKG) which is a graphic recordation of cardiac activity, either on paper or a computer monitor. An ECG can be beneficial in detecting disease and/or damage; (iii) echocardiogram (heart ultrasound) used to investigate congenital heart disease and assessing abnormalities of the heart wall, including functional abnormalities of the heart wall, valves and blood vessels; (iv) Doppler ultrasound can be used to measure blood flow across a heart valve; (v) nuclear medicine imaging (also referred to as radionuclide scanning in the art) allows visualization of the anatomy and function of an organ, and can be used to detect coronary artery disease, myocardial infarction, valve disease, heart transplant rejection, check the effectiveness of bypass surgery, or to select patients for angioplasty or coronary bypass graft.

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

As used herein, the terms “administering,” “introducing” and “transplanting” are used interchangeably and refer to the placement of the cardiac myocytes as described herein into a subject by a method or route which results in at least partial localization of the cardiovascular stem cells at a desired site. The cardiovascular stem cells can be administered by any appropriate route which results in effective treatment in the subject, e.g. administration results in delivery to a desired location in the subject where at least a portion of the cells or components of the cells remain viable. The period of viability of the cells after administration to a subject can be as short as a few hours, e. g. twenty-four hours, to a few days, to as long as several years.

The phrases “parenteral administration” and “administered parenterally” as used herein mean modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of atrial progenitors or atrial myocytes and/or their progeny and/or compound and/or other material other than directly into the cardiac tissue, such that it enters the animal's system and, thus, is subject to metabolism and other like processes.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. The pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable ingredients. The carrier can be in the form of a solid, semi-solid or liquid diluent, cream or a capsule.

The terms “composition” or “pharmaceutical composition” used interchangeably herein refer to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, ocular parenteral, intravenous, intraarterial, subcutaneous, intranasal, sublingual, intraspinal, intracerebroventricular, and the like. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

The term “drug” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “agent” refers to any entity which is normally not present or not present at the levels being administered to a cell, tissue or subject. Agent can be selected from a group comprising: chemicals; small molecules; nucleic acid sequences; nucleic acid analogues; proteins; peptides; aptamers; antibodies; or functional fragments thereof. A nucleic acid sequence can be RNA or DNA, and can be single or double stranded, and can be selected from a group comprising: nucleic acid encoding a protein of interest; oligonucleotides; and nucleic acid analogues; for example peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. An gent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The articles “a” and an are used herein to refer to one or to more than one (e.g., to at least one) of the grammatical object of the article. By way of example, an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean ±1%. The present invention is further explained in detail by the following examples, but the scope of the invention should not be limited thereto.

As used herein, the term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the present invention related to the herein described compositions, methods, and respective component(s) thereof, as essential to the invention, yet open to the inclusion of unspecified elements, essential or not (“comprising”).

The term “consisting essentially of means “including principally, but not necessary solely at least one”, and as such, is intended to mean a “selection of one or more, and in any combination.” Stated another way, other elements can be included in the description of the composition, method or respective component thereof provided the other elements are limited to those that do not materially affect the basic and novel characteristic(s) of the invention (“consisting essentially of”). This applies equally to steps within a described method as well as compositions and components therein.

The term “consisting of” as used herein as used in reference to the inventions, compositions, methods, and respective components thereof, is intended to be exclusive of any element not deemed an essential element to the component, composition or method.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Cardiac Progenitor Cells (CPC)

As disclosed herein, using transgenic using a two color system and fluorescently activated cell sorting (FACS) sorting, the inventors have identified and isolated discrete populations of cardiac progenitor cells which represent a sub-populations of first heart field (FHF) and second heart field (SHF) progenitors. In particular, the inventors have identified and isolated three distinct unique populations of cardiac progenitors: (1) double labeled dsRed +/eGFP+(R+G+) population representing second heart field (SHF) progenitors which are committed to the right ventricle (RV) and outflow tract (OFT) progenitors, and herein is referred to as a committed ventricular progenitor (CVP), (2) single labeled dsRed+ negative (referred to herein as dsRed +/eGFP− or R+G−) population representing second heart field (SHF) progenitors which are committed to primitive isl1+ pharyngeal mesoderm (PM) progenitors, and (3) single labeled eGFP+ (referred to herein as dsRed−/eGFP+ or R−G+) population representing first heart field (FHF) progenitors which are committed to the left ventricle (LV) and inflow tract progenitors. These progenitors were compared to the reference non-cardiac progenitors which expressed neither dsRed nor eGFP (referred to herein as dsRed−/eGFP− or R−G−).

Accordingly, one aspect of the invention relates to the identification, isolation and characterization of a sub-population of second heart field (SHF) progenitors referred to herein as committed ventricular progenitor (CVP) cells, which are committed differentiating into right ventricle (RV) and outflow tract (OFT) progenitors which also give rise to ventricular cardiomyocytes. In particular the present invention provides methods for isolating CVP cells capable of contributing to ventricular myocardium, in particular to functional ventricular myocardium. A CVP cell can be identified by expression of at least two, or at least 3 of the following positive markers selected from the group comprising; Mef2c, Nkx2.5, Tbx20, Isl1+, GATA4, and GATA6; myocardial markers Tropinin T, Troponin C, BMP signalling molecules; BMP7, BMP4, BMP2 and miRNA molecules; miR-208, miR-143, miR-133a, miR-133b, miR-1, miR-143, miR-689. Furthermore, in combination with at least two or more of the above-listed positive expression markers, a CVP cell can be identified by their lack of, or low level expression of the following negative markers; the primary heart field marker Tbx5, and other markers, such as Snai2, miR-200a, miR-200b, miR-199a, miR-199b, miR-126-3p, miR-322, CD31. Furthermore, the identification of CVP (R+G+) can be distinguished from other secondary heart progenitors, such as dsRed+/eGFP−(R+G−) or first heart field progenitors (i.e dsRed−/eGFP+, R−G+) based on the molecular marker profile as disclosed in Table 1.

Accordingly, also encompassed within the scope of the present invention are methods for the identification and isolation of such committed ventricular progenitor (CVP) cells by at least one agent which is reactive to at least Mef2c and Nkx2.5. One of ordinary skill in the art can identify and isolate a CVP cell as disclosed herein using agents reactive to any combination of positive and/or negative markers listed in Table 1. For example, a cell or population of cells which react positively the expression of Mef2c, Nkx2.5 and Isl1 can identify CVP (dsRed+/eGFP+, R+G+) cells, which can be distinguished from cells which react positively to the expression of Mef2c and Isl1, but negative for the expression of Nkx2.5, and thus identify dsRed+/eGFP−, R+G−) cells.

In one embodiment, an agent which is reactive to one of the markers listed in Table 1 is an agent which react to the nucleic acid encoding such marker protein, for example an agent can specifically hybridize under stringent conditions to nucleic acids, such as mRNA encoding a marker polypeptide. In other embodiments, an agent which is reactive to one of the markers listed in Table 1 is an agent which react to the marker protein, for example an agent can specifically bind to a marker protein, or fragment thereof. Another embodiment encompasses methods for the identification and/or isolation of CVP cells comprising Mef2c and Nkx2.5 markers using a marker or reporter gene, as those terms are defined herein, which is operatively linked to a promoter or region thereof which controls the transcription of the Mef2c gene, and a promoter or region thereof which controls the transcription of Nkx2.5 or homologues or variants thereof, as disclosed in the Examples herein. By way of a non-limiting example and as disclosed herein, the inventors demonstrate identification and isolation of CVP cell by FACS and selecting for cells which express both DsRed and eGFP (R+G+), where DsRed is a reporter gene operatively linked to the Mef2c promoter and where the eGFP is a reporter gene operatively linked to the Nkx2.5 promoter. Therefore when DsRed is expressed, it concomitantly identifies the expression of the Mef2c gene, and similarly when eGFP is expressed, it concomitantly identifies the expression of the eGFP gene.

In some embodiments, a CVP cell can be identified and isolated by using agents reactive to other markers typical of the CVP lineage, including but without limitation those which are disclosed in Table 1. For example, a CVP cell in a population of cells can be selected based on the positive expression of Mef2c and Nxk2.5 and at least one of the following positive markers; Tbx20, Isl1, GATA4, GATA6; Tropinin T (TnT), Troponin C (TnI), BMP7, BMP4, BMP2, miR-208, miR-143, miR-133a, miR-133b, miR-1, miR-143, miR-689 and smooth muscle actin (smActin), or homologues or variants thereof. Alternatively, a CVP cell in a population of cells can be selected based on the positive expression of Mef2c and Nxk2.5 and the negative expression of a negative marker gene including but without limitation those as disclosed in Table 1. For example, a CVP cell in a population of cells can be selected based on the positive expression of Mef2c and Nxk2.5 and at least one of the negative marker, where the cell lacks the expression or has low level expression of at least one of the following markers; Tbx5, Snai2, miR-200a, miR-200b, miR-199a, miR-199b, miR-126-3p, miR-322 and CD31 or homologues or variants thereof.

Typically, conventional methods to isolate a CVP cell involves positive and negative selection using markers of interest. For example, agents can be used to recognize markers present on the CVP cells, for instance labeled antibodies that recognize and bind to cell-surface markers or antigens on a CVP cell which can be used to separate and isolate a CVP cell from a population of non-CVP cells using fluorescent activated cell sorting (FACS), panning methods, magnetic particle selection, particle sorter selection and other methods known to persons skilled in the art, including density separation (Xu et al. (2002) Circ. Res. 91:501; U.S. patent application Ser. No. 20030022367) and separation based on other physical properties (Doevendans et al. (2000) J. Mol. Cell. Cardiol. 32:839-851). Alternatively, genetic selection methods can be used, where a CVP cell can be genetically engineered to express a reporter protein operatively linked to a tissue-specific promoter and/or a specific gene promoter, therefore the expression of the reporter can be used for positive selection methods to isolate and enrich for a population of CVP cells. For example, a fluorescent reporter protein can be expressed in the desired stem cell by genetic engineering methods to operatively link the marker protein to the promoter expressed in a desired stem cell (Klug et al. (1996) J. Clin. Invest. 98:216-224; U.S. Pat. No. 6,737,054). Other means of positive selection include drug selection, for instance such as described by Klug et al, supra, involving enrichment of desired cells by density gradient centrifugation. Negative selection can be performed and selecting and removing cells with undesired markers or characteristics, for example fibroblast markers, epithelial cell markers etc.

In some embodiments, isolation of CVP cells comprises a separation step involving contacting a heterologous population of cells (e.g. CVP cells and non-CVP cells) with an antibody specific for at least one, or at least two or at least three CVP-specific markers.

Separation can be carried out using any of a number of well-known methods, including, e.g., any of a variety of sorting methods, e.g., fluorescence activated cell sorting (FACS), negative selection methods, etc. The selected cells are separated from non-selected cells, generating a population of selected (“sorted”) cells. A selected cell population can be at least about 75%, at least about 80%, at least about 85%, at least about 90%, at least about 95%, at least about 98%, at least about 99%, or greater than 99% cardiomyocytes.

Cell sorting (separation) methods are well known in the art. Procedures for separation may include magnetic separation, using antibody-coated magnetic beads, affinity chromatography and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. Dead cells may be eliminated by selection with dyes associated with dead cells (propidium iodide [PI] LDS). Any technique may be employed which is not unduly detrimental to the viability of the selected cells. Where the selection involves use of one or more antibodies, the antibodies can be conjugated with labels to allow for ease of separation of the particular cell type, e.g. magnetic beads; biotin, which binds with high affinity to avidin or streptavidin; fiuorochromes, which can be used with a fluorescence activated cell sorter; haptens; and the like. Multi-color analyses may be employed with the FACS or in a combination of immunomagnetic separation and flow cytometry.

In some embodiments, the CVP cells as disclosed herein can differentiate into mature ventricular cardiomyocytes, and can develop into functional ventricular tissue which comprises spontaneous periodic contractile activity. In some embodiments, the functional ventricular tissue can be evoked to contract upon appropriate stimulation. Spontaneous contraction generally means that, when cultured in a suitable tissue culture environment with an appropriate Ca²⁺ concentration and electrolyte balance, the cells can be observed to contract in a periodic fashion across one axis of the cell, and then release from contraction, without having to add any additional components to the culture medium. Non-spontaneous contraction may be observed, for example, in the presence of pacemaker cells, or other stimulus.

TABLE 1
Summary of markers expressed by the three cardiac progenitors identified herein; (i)
dsRed+/eGFP+ (R+G+) (CVP cells) (ii) dsRed+/eGFP− (R+G−) (iii) and dsRed−/eGFP+ (R+G−)

| | Cardiac | Give rise | Differentiate | POSITIVE | |
|---|---|---|---|---|---|
| | progenitor | to heart | into tissue and | expression | NEGATIVE |
| Lineage | subtype | structures: | cell types: | Markers | expression Markers |
| SHF | dsRed+/eGFP+ | RV and | Ventricular | Mef2c, Nkx2.5, | Tbx5, Snai2, miR- |
| | (R+G+) | OFT | cardiomyocytes | Tbx20, Isl1, | 200a, miR-200b, |
| | (Committed | | | GATA4, GATA6; | miR-199a, miR- |
| | ventricular | | | Tropinin T (TnT), | 199b, miR-126-3p, |
| | progenitors | | | Troponin C (TnI), | miR-322, CD31 |
| | (CVP) cells) | | | BMP7, BMP4, | |
| | | | | BMP2, miR-208, | |
| | | | | miR-143, miR- | |
| | | | | 133a, miR-133b, | |
| | | | | miR-1, miR-143, | |
| | | | | miR-689, smActin | |
| Primitive | dsRed+/eGFP− | PM | Endothelial cells, | Mef2c, Isl1, | Nkx2.5, TnT, TnI, |
| SHF | (R+G−) | | smooth muscle | Snai2, | Tbx5, GATA4, |
| | | | cells and cardiac | miRNA199a, | Tbx20, CD31, |
| | | | muscle cells | miRNA199b, | BMP2, smActin, |
| | | | | BMP4 | miR-200a, miR- |
| | | | | | 200b, miR-143, miR- |
| | | | | | 133a, miR-1 |
| FHF | dsRed−/eGFP+ | LV and | Smooth muscle | Nkx2.5, Tbx5, | Mef2c, Isl1, Tbx20, |
| | (R−G+) | inflow tract | and cardiac | TnT, TnI, GATA6, | Snai2, BMP4, CD31, |
| | | | myocytes | GATA4, BMP7, | miR-199a, miR- |
| | | | | BMP2, smActin, | 199b, miR-322, miR- |
| | | | | miRNA200a, | 143 |
| | | | | miRNA200b, | |
| | | | | miR-126-3p, miR- | |
| | | | | 208, miR-133a, | |
| | | | | miR-1 | |
| Non- | dsRed−/eGFP− | Non- | | | Mef2c, Nkx2.5, Isl1, |
| cardiac | (R−G−) | cardiac | | | CD31, Tbx5, Snai2, |
| | | progenitors | | | BMP7, BMP5, |
| | | | | | BMP4, BMP2 |

Methods to Identify and Isolate CVP Cells

Methods to determine the expression, for example the expression of RNA or protein expression of markers of CVP cells as disclosed herein, such as Mef2c and Nkx2.5 expression are well known in the art, and are encompassed for use in this invention. Such methods of measuring gene expression are well known in the art, and are commonly performed on using DNA or RNA collected from a biological sample of the cells, and can be performed by a variety of techniques known in the art, including but not limited to, PCR, RT-PCR, quantitative RT-PCR (qRT-PCR), hybridization with probes, northern blot analysis, in situ hybridization, microarray analysis, RNA protection assay, SAGE or MPSS. In some embodiments, the probes used detect the nucleic acid expression of the marker genes can be nucleic acids (such as DNA or RNA) or nucleic acid analogues, for example peptide-nucleic acid (PNA), pseudocomplementary PNA (pcPNA), locked nucleic acid (LNA) or analogues or variants thereof.

In other embodiments, the expression of the markers can be detected at the level of protein expression. The detection of the presence of nucleotide gene expression of the markers, or detection of protein expression can be similarity analyzed using well known techniques in the art, for example but not limited to immunoblotting analysis, western blot analysis, immunohistochemical analysis, ELISA, and mass spectrometry. Determining the activity of the markers, and hence the presence of the markers can be also be done, typically by in vitro assays known by a person skilled in the art, for example Northern blot, RNA protection assay, microarray assay etc of downstream signaling pathways of Mef2c and Nkx2.5. In particular embodiments, qRT-PCR can be conducted as ordinary qRT-PCR or as multiplex qRT-PCR assay where the assay enables the detection of multiple markers simultaneously, for example Mef2c and/or Nkx2.5., either together or separately from the same reaction sample.

One variation of the RT-PCR technique is the real time quantitative PCR, which measures PCR product accumulation through a dual-labeled fluorigenic probe (e.g., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996). Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994. TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™ The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data. 5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct). To minimize errors and the effect of sample-to-sample variation, RT-PCR is usually performed using an internal standard. The ideal internal standard is expressed at a relatively constant level among different tissues, and is unaffected by the experimental treatment. RNAs frequently used to normalize patterns of gene expression are mRNAs for the housekeeping genes glyceraldehyde-3-phosphate-dehydrogenase (GAPDH) and β-actin.

In some embodiments, the systems for real-time PCR uses, for example, Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the sequence of interest are generally sufficient. In addition, a standard curve is generated for the control sequence. This permits standardization of initial content of the nucleic acid of interest in a tissue sample to the amount of control for comparison purposes.

Other methods for detecting the expression of the marker gene are well known in the art and disclosed in patent application WO/200004194, incorporated herein by reference. In an exemplary method, the method comprises amplifying a segment of DNA or RNA (generally after converting the RNA to cDNA) spanning one or more known isoforms of the markers (such as Isl-1, Nkx2.5, flk1) gene sequences. This amplified segment is then subjected to a detection method, such as signal detection, for example fluorescence, enzymatic etc. and/or polyacrylamide gel electrophoresis. The analysis of the PCR products by quantitative mean of the test biological sample to a control sample indicates the presence or absence of the marker gene in the cardiovascular stem cell sample. This analysis may also be performed by established methods such as quantitative RT-PCR (qRT-PCR).

The methods of RNA isolation, RNA reverse transcription (RT) to cDNA (copy DNA) and cDNA or nucleic acid amplification and analysis are routine for one skilled in the art and examples of protocols can be found, for example, in the Molecular Cloning: A Laboratory Manual (3-Volume Set) Ed. Joseph Sambrook, David W. Russel, and Joe Sambrook, Cold Spring Harbor Laboratory; 3rd edition (Jan. 15, 2001), ISBN: 0879695773. Particularly useful protocol source for methods used in PCR amplification is PCR (Basics: From Background to Bench) by M. J. McPherson, S. G. Moller, R. Beynon, C. Howe, Springer Verlag; 1st edition (Oct. 15, 2000), ISBN: 0387916008. Other methods for detecting expression of the marker genes by analyzing RNA expression comprise methods, for example but not limited to, Northern blot, RNA protection assay, hybridization methodology and microarray assay etc. Such methods are well known in the art and are encompassed for use in this invention.

Primers specific for PCR application can be designed to recognize nucleic acid sequence encoding Mef2c and Nkx2.5, are well known in the art. For purposes of an example only, the nucleic acid sequence encoding human Mef2c can be identified by accession number: AL833268 (SEQ ID NO:1) or NM_{—}002397 (SEQ ID NO:2). For purposes of an example, the nucleic acid sequence encoding human Nkx2.5 can be identified by GenBank Accession No: AB021133 (SEQ ID NO:3) or NM_{—}004387 (SEQ ID NO: 4).

Nkx2-5 is a cardiac transcription factor that binds the atrial natriuretic factor promoter. Durocher et al. (1997) EMBO J. 16:5687. Amino acid sequences of Nkx2-5 polypeptides are known in the art. See, e.g., Turbay et al. (1996) MoI. Med. 2:86; GenBank Accession No. NP_{—}004378 {Homo sapiens Nkx2-5); GenBank Accession No. AAC97934; Mus musculus Nkx2-5); and GenBank Accession No. AAB62696 (Rattus norvegicus Nkx2-5). Nkx2-5 polypeptides include a polypeptide having at least about 75%, at least about 80%, at least about 85%, at least about 90%, at least about 95%, at least about 98%, at least about 99%, or 100%, amino acid sequence identity to the amino acid sequence set forth in GenBank Accession No. NP_{—}004378. The term “Nkx2-5 polypeptide” includes polypeptides having at least about 75%, at least about 80%, at least about 85%, at least about 90%, at least about 95%, at least about 98%, at least about 99%, or 100%, amino acid sequence identity over a contiguous stretch of from about 300 amino acids to about 305 amino acids, from about 305 amino acids to about 310 amino acids, from about 310 amino acids to about 315 amino acids, or from about 315 amino acids to about 324 amino acids. An Nkx2-5 polypeptide can have a length of from about 300 amino acids to about 305 amino acids, from about 305 amino acids to about 310 amino acids, from about 310 amino acids to about 315 amino acids, from about 315 amino acids to about 318 amino acids, or from about 318 amino acids to about 324 amino acids.

The term “Nkx2-5 polypeptide” includes fusion polypeptides comprising a Nkx2-5 polypeptide and a non-Nkx2-5 polypeptide (e.g., a “fusion partner” or a “heterologous polypeptide”). Suitable fusion partners include, e.g., epitope tags, proteins that provide a detectable signal; proteins that aid in purification; and the like, as described in more detail below.

An “Nkx2-5 nucleic acid” comprises a nucleotide sequence encoding an Nkx2-5 polypeptide. Nucleotide sequences encoding Nkx2-5 polypeptides are known in the art. See, e.g., GenBank Accession No. NM_{—}004387 (encoding a Homo sapiens Nkx2-5 polypeptide); GenBank Accession No. AF091351 (encoding a Mus musculus Nkx2-5 polypeptide); and GenBank Accession No. AF006664 (encoding a Rattus norvegicus Nkx2-5 polypeptide). Nkx2-5 nucleic acids suitable for use in a subject method include a nucleic acid comprising a nucleotide sequence having at least about 75%, at least about 80%, at least about 85%, at least about 90%, at least about 95%, at least about 98%, at least about 99%, or 100%, nucleotide sequence identity to a contiguous stretch of from about 900 nucleotides to about 925 nucleotides, from about 925 nucleotides to about 950 nucleotides, or from about 950 nucleotides to about 975 nucleotides.

Any suitable immunoassay format known in the art and as described herein can be used to detect the presence of and/or quantify the amount of marker, for example Mef2c or Nkx2.5, markers expressed by the cardiovascular stem cell. The invention provides a method of screening for the markers expressed by a CVP or population of CVP cells by immunohistochemical or immunocytochemical methods, typically termed immunohistochemistry (“IHC”) and immunocytochemistry (“ICC”) techniques. IHC is the application of immunochemistry on samples of tissue, whereas ICC is the application of immunochemistry to cells or tissue imprints after they have undergone specific cytological preparations such as, for example, liquid-based preparations. Immunochemistry is a family of techniques based on the use of a specific antibody, wherein antibodies are used to specifically recognize and bind to target molecules on the inside or on the surface of cells, for example Mef2c and/or Nkx2.5. In some embodiments, the antibody contains a reporter or marker that will catalyze a biochemical reaction, and thereby bring about a change color, upon encountering the targeted molecules. In some instances, signal amplification may be integrated into the particular protocol, wherein a secondary antibody, that includes the marker stain, follows the application of a primary specific antibody. In such embodiments, the marker is an enzyme, and a color change occurs in the presence and after catalysis of a substrate for that enzyme.

Immunohistochemical assays are known to those of skill in the art (e.g., see Jalkanen, et al., J. Cell. Biol. 101:976-985 (1985); Jalkanen, et al., J. Cell. Biol. 105:3087-3096 (1987). Antibodies, polyclonal or monoclonal, can be purchased from a variety of commercial suppliers, or may be manufactured using well-known methods, e. g., as described in Harlow et al., Antibodies: A Laboratory Manual, 2nd Ed; Cold. Spring Harbor Laboratory Press, Cold Spring Harbor, N.Y. (1988). In general, examples of antibodies useful in the present invention include anti-Islet1 or anti-SLN antibodies. Such antibodies can be purchased, for example, from Developmental Hybridoma Bank; BD PharMingen; Biomedical Technologies; Sigma; RDI; Roche and other commercially available sources. Alternatively, antibodies (monoclonal and polyclonal) can easily produced by methods known to person skilled in the art. In alternative embodiments, the antibody can be an antibody fragment, an analogue or variant of an antibody. In some embodiments, any antibodies that recognize Mef2c or Nkx2.5 can be used by any persons skilled in the art, and from any commercial source.

For detection of the makers by immunohistochemistry, the CVP cells may detected using antibodies which are labeled and can be subsequently FAC sorted according to methods known by a person of ordinary skill in the art. Commercially available antibodies can be used, and can be purchased from companies such as Cell Signalling, ABI, sigma, Stressgen, SantaCruz Biotechnology AbCam, Ad Serotec, Invitrogen and the like.

In some embodiments, the CVP cells are fixed prior to immunodetection by a suitable fixing agent such as alcohol, acetone, and paraformaldehyde prior to, during or after being reacted with (or probed) with an antibody. Conventional methods for immunohistochemistry are described in Harlow and Lane (Eds) (1988) In “Antibodies A Laboratory Manual”, Cold Spring Harbor Press, Cold Spring Harbor, N.Y.; Ausbel et al (Eds) (1987), in Current Protocols In Molecular Biology, John Wiley and Sons (New York, N.Y.). Biological samples appropriate for such detection assays include, but are not limited to, cells, tissue biopsy, whole blood, plasma, serum, sputum, cerebrospinal fluid, breast aspirates, pleural fluid, urine and the like. For direct labeling techniques, a labeled antibody is utilized. For indirect labeling techniques, the sample is further reacted with a labeled substance. Alternatively, immunocytochemistry may be utilized. In general, cells are obtained from a patient and fixed by a suitable fixing agent such as alcohol, acetone, and paraformaldehyde, prior to, during or after being reacted with (or probed) with an antibody. Methods of immunocytological staining of biological samples, including human samples, are known to those of skill in the art and described, for example, in Brauer et al., 2001 (FASEB J, 15, 2689-2701), Smith Swintosky et al., 1997. Immunological methods of the present invention are advantageous because they require only small quantities of biological material, such as a small quantity of cardiovascular stem cells. Such methods may be done at the cellular level and thereby necessitate a minimum of one cell.

In some embodiments, cells can be permeabilized to stain cytoplasmic molecules. In general, antibodies that specifically bind a differentially expressed polypeptide are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.) The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

In a different embodiment, antibodies (a term that encompasses all antigen-binding antibody derivatives and antigen-binding antibody fragments) that recognize the markers Mef2c or Nkx2.5 are used to detect cells that express the markers. The antibodies bind at least one epitope on one or more of the markers and can be used in analytical techniques, such as by protein dot blots, sodium dodecyl sulfate polyacrylamide gel electrophoresis (SDS-PAGE), or any other gel system that separates proteins, with subsequent visualization of the marker (such as Western blots). Antibodies can also be used, for example, in gel filtration or affinity column purification, or as specific reagents in techniques such as fluorescent-activated cell sorting (FACS). Other assays for cells expressing a specific marker can include, for example, staining with dyes that have a specific reaction with a marker molecule (such as ruthenium red and extracellular matrix molecules), identification specific morphological characteristics (such as the presence of microvilli in epithelia, or the pseudopodialfilopodia in migrating cells, such as fibroblasts and mesenchyme). Biochemical assays include, for example, assaying for an enzymatic product or intermediate, or for the overall composition of a cell, such as the ratio of protein to lipid, or lipid to sugar, or even the ratio of two specific lipids to each other, or polysaccharides. If such a marker is a morphological and/or functional trait or characteristic, suitable methods including visual inspection using, for example, the unaided eye, a stereomicroscope, a dissecting microscope, a confocal microscope, or an electron microscope are encompassed for use in the invention. The invention also contemplates methods of analyzing the progressive or terminal differentiation of a cell employing a single marker, as well as any combination of molecular and/or non-molecular markers.

Various methods can be utilized for quantifying the presence of the selected markers and or reporter gene. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

Also encompassed for use in this invention and as disclosed in the Examples is the isolation of CVP cells by the use of an introduced reporter gene that aids with the identification and selection of CVP cells from a mixed population of CVP cells and non-CVP cells. For example, a CVP cell can be genetically engineered to express a construct comprising a reporter gene which can be used for selection and identification purposes. For example, a CVP cell or population of CVP cells can be genetically engineered to comprise a reporter gene, for example but not limited to a fluorescent protein, enzyme or resistance gene, which is operatively linked to a particular promoter (for example, but not limited to Mef2c and/or Nkx2.5). In such an embodiment, when the cell expresses the gene to which the reporter of interest is operatively linked, it also expresses the reporter gene, for example the enzyme, fluorescent protein or resistance gene. Cells that express the reporter gene can be readily detected and in some embodiments positively selected for cells comprising the reporter gene or the gene product of the reporter gene. Other reporter genes that can be used include fluorescent proteins, luciferase, alkaline phosphatase, lacZ, or CAT.

This invention also encompasses the generation of useful clonal reporter cell lines, such as embryonic stem (ES) cell lines as disclosed herein, where a ES cell line is genetically altered to comprise multiple reporter genes to aid in the identification of ES cell that has differentiated along to become a CVP cell. Cells expressing these reporters could be easily purified by FACS, antibody affinity capture, magnetic separation, or a combination thereof. The purified or substantially pure population of CVP cells, such as EP-derived CVP cells as disclosed herein can be used for genomic analysis by techniques such as microarray hybridization, SAGE, MPSS, or proteomic analysis to identify more markers that characterize the CVP cells. These methods are also useful to identify secondary heart field (SHF) progenitors which are not CVP cells, or progeny of CVP cells which have not differentiated into ventricular cardiomyocyte cells.

In some embodiments, a reporter gene is a resistance gene, the resistance gene can be, for example but not limited to, genes for resistance to amplicillin, chloroamphenicol, tetracycline, puromycin, G418, blasticidin and variants and fragments thereof, which can be used as a functional positive selection marker to select for a population of CVPs, where the non-CVP cells do not express the resistance gene. In other embodiments, the reporter gene can be a fluorescent protein, for example but not limited to: green fluorescent protein (GFP); green fluorescent-like protein (GFP-like); yellow fluorescent protein (YFP); blue fluorescent protein (BFP); enhanced green fluorescent protein (EGFP); enhanced blue fluorescent protein (EBFP); cyan fluorescent protein (CFP); enhanced cyan fluorescent protein (ECFP); red fluorescent protein (dsRED); and modifications and fluorescent fragments thereof.

In some embodiments, methods to remove unwanted cells are encompassed, by removing unwanted cells by negative selection. For example, unwanted antibody-labeled cells are removed by methods known in the art, such as labeling a cell population with an antibody or a cocktail of antibodies, to a cell surface protein and separation by FACS or magnetic colloids. In an alternative embodiment, the reporter gene may be used to negatively select non-desired cells, for example a reporter gene encodes a cytotoxic protein in cells that are not desired. In such an embodiment, the reporter gene is operatively linked to a regulatory sequence of a gene normally expressed in the cells with undesirable phenotype.

One embodiment of the invention provides a substantially pure population of CVP cells. In some embodiments, the substantially pure population of CVP cells can be used in the generation of functional tissue engineered myocardium as disclosed herein, where a substantially pure population of CVP cells is seeded on an appropriate scaffold, such as polydimehylsiloxane (PDMS) elastomer substrate for the generation of a muscle thin film (MTF) as disclosed herein.

Accordingly, one aspect of the present invention relates to the use of the CVP in the generation of functional myocardium tissue. In particular, one aspect of the present relates to a composition comprising the tissue engineered myocardium as disclosed herein, comprising a scaffold and a substantially pure population of committed ventricular progenitor (CVP) cells which are capable of giving rise to mature ventricular cardiomyocytes. Accordingly, a substantially pure population of committed ventricular progenitors (CVPs) on an appropriate scaffold can result in a mature strip of fully functional cardiac muscle tissue, herein referred to a muscular thin film (MTF).

In some embodiments, the CVP cells for use in the MTF or for the generation of tissue engineered myocardium are of mammalian origin, and in some embodiments the CVP cells are of human origin. In other embodiments, a population of CVP cells are or rodent origin, for example mouse, rat or hamster. In another embodiment, a population of CVP cells for use is a genetically engineered CVP cell, for example where the CVP has been genetically modified to carry a pathological gene which causes, or increases the risk of a cardiovascular disease. Alternatively, a CVP can been genetically modified to have a functional characteristic of a cardiovascular disease, for instance the CVP exhibits a phenotype of a cardiovascular disease. By way of a non-limiting example, a CVP which has a characteristic or phenotype of a cardiovascular disease can be, for example, but not limited to, a decrease in spontaneous contraction, or decrease or increase in contractile force etc.

Sources of CVP Cells

As discussed above, one embodiment of the present invention is a tissue engineered myocardial composition comprising a substantially pure population of CVP cells seeded on a substrate. In another embodiment, the invention provides methods for the generation of functional tissue engineered myocardium as disclosed herein, comprising a substantially pure population of CVP cells seeded on an appropriate scaffold, such as polydimehylsiloxane (PDMS) elastomer substrate for the generation of a muscle thin film (MTF) as disclosed herein.

As disclosed herein in the Examples, the inventors have demonstrated the use of ES cell derived-CVPs and tissue derived-CVP cells in the generation of MTF. Accordingly, one can use CVP cells derived from tissues, such as embryonic cardiac tissue and/or ES cell sources for use in the generation of functional tissue engineered myocardium as disclosed herein. Alternatively, one can use CVP cells derived from any number of cells sources known to a person of ordinary skill in the art, such as for example, but not limited to, stem cells, such as cardiac progenitor cells, or embryonic sources, embryonic stem (ES) cells, adult stem cells (ASC), embryoid bodies (EB) and iPS cells. In some embodiments, an iPS cell produced by any method known in the art can be used, for example virally-induced or chemically induced generation of iPS cells as disclose in EP1970446, US2009/0047263, US2009/0068742, and 2009/0227032, which are incorporated herein in their entirety by reference. In some embodiments CVP cells are derived from human embryonic stem cell lines.

For example, CVP cells as disclosed herein can be derived from Isl1+ multipotent progenitor cells such as those previously isolated and identified by the inventors and disclosed in U.S. Provisional Application 60/856,490 and 60/860,354 and in International Application PCT/US07/23155, which is incorporated herein in its entirety by reference.

Accordingly, CVP cells for use in the methods and compositions as disclosed herein can be any cells derived from any kind of tissue or cell line, such as a stem cell line (for example embryonic tissue such as fetal or pre-fetal tissue, or adult tissue), where the CVP cells have the characteristic of being capable of producing ventricular cardiomyocytes. Such cells used to derive CVP cells can be provided in the form of an established cell line, or they may be obtained directly from primary embryonic tissue and used immediately for differentiation. Included are human embryonic stem cell lines, such as thoses listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, CVP cells use in the methods and compositions as disclosed herein are derived from a stem cell source where the embryo is not destroyed.

In another embodiment, a CVP cell for use in the methods and tissue engineered myocardium as disclosed herein can be isolated from tissue including solid tissues, such as cardiac tissue including cardiac muscle (the exception to solid tissue is whole blood, including blood, plasma and bone marrow). In some embodiments, the tissue is heart or cardiac tissue. In other embodiments, the tissue is for example but not limited to, umbilical cord blood, placenta, bone marrow, or chondral villi. Stem cells of interest which can be used to derive CVP cells also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.)

In some embodiments, CVP cells for use in the methods and tissue engineered myocardium as disclosed herein can may be derived from tissues or stem cells obtained from any mammalian species, e.g. human, equine, bovine, porcine, canine, feline, rodent, e.g. mice, rats, hamster, primate, etc. In some embodiments, the CVP cells for use in the methods and compositions as disclosed herein are human CVP cells.

Without wishing to be bound by theory, ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-I-60, TRA-1-81, and alkaline phosphatase. The globo-series glycolipid GL7, which carries the SSEA-4 epitope, is formed by the addition of sialic acid to the globo-series glycolipid Gb5, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920 which are incorporated herein by reference.

In some embodiments, the CVP is derived from a human embryonic stem cell. In some embodiments, a generation of the CVP, and embryo is not destroyed. Human embryonic stem cells that are suitable for use include, but are not limited to, BGO1, BG02, and BG03 (provider's code hESBGN-01, hESBGN-02, and hESBGN-03, respectively) (BresaGen, Inc.); SAO1 and SA02 (provider's code Sahlgrenska 1 and Sahlgrenska 2, respectively) (Cellartis AB); ESO1, ES02, ES03, ES04, ES05, and ES06 (provider's code HES-I, HES-2, HES-3, HES-4, HES-5, and HES-6, respectively) (ES Cell International); TE03, TE04, and TE06 (provider's code 1 3, 1 4, and I 6, respectively) (National Stem Cell Bank); UCO1 and UC06 (provider's code HSF-I and HSF-6, respectively) (University of California, San Francisco); WAO1, WA07, WA09, WA13, and WA17 (provider's code H1, H7, H9, H13, and H 14, respectively) (Wisconsin Alumni Research Foundation, WiCeIl Research Institute). In some embodiments, a human embryonic stem cell has the following characteristics: SSEA-I+, SSEA-2⁺, SSEA-3⁺, SSEA-4⁺, TRA 1-60⁺, TRA 1-8I⁺, Oct-4⁺, and alkaline phosphatase (AP+). Methods of isolating human embryonic cell cells are known in the art. See, e.g., U.S. Pat. No. 7,294,508 which is incorporated herein by reference.

In some embodiments, CVP cells can be derived from hematopoietic stem cells, or from a suitable source of endothelial, muscle, and/or neural stem cells which are harvested from a mammalian donor by methods known by one of ordinary skill in the art. A suitable source is the hematopoietic microenvironment. For example, circulating peripheral blood, preferably mobilized (e.g., recruited) as described below, may be removed from a subject. Alternatively, bone marrow may be obtained from a mammal, such as a human patient, undergoing an autologous transplant.

In alternative embodiments, CVP cell for use in the methods., compositions and tissue engineered myocardium as disclosed herein can be derived from human umbilical cord blood cells (HUCBC) have recently been recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (e.g. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79;1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985 J. Immunol. 134:1493-1497).

Without wishing to be bound by theory, human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79;1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497 Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096). One source of cells is the hematopoietic micro-environment, such as the circulating peripheral blood, preferably from the mononuclear fraction of peripheral blood, umbilical cord blood, bone marrow, fetal liver, or yolk sac of a mammal. A CVP cell for use in the methods and tissue engineered myocardium as disclosed herein can be derived from stem cells such as neural stem cells or stem cells derived from the central nervous system, including the meninges.

In an alternative embodiment, a population of CVP cells for use in the methods and tissue engineered myocardium as disclosed herein can be de-differentiated stem cells, such as stem cells derived from differentiated cells. In such an embodiment, the de-differentiated stem cells can be for example, but not limited to, neoplastic cells, tumor cells and cancer cells. In some embodiments, the de-differentiated cells are from a subject, such as a human subject. In some embodiments, the subject such as a human subject has, or is at risk of developing a cardiovascular disease or condition, or the subject has a cardiac pathology or cardiomyopathy. In some embodiments, the subject is a human subject in need of a cardiac treatment and the subject derived-CVP cells are used to generate a tissue engineered myocardium as disclosed herein which is transplanted into the same subject in which the cells were obtained to derive the CVP cells. In some embodiments, the de-differentiated stem cells are obtained from a biopsy.

In some embodiments, the CVP cells are derived from the reprogramming of cells. For example, a population of CVP cells for use in the methods and tissue engineered myocardium as disclosed herein can be from an induced pluripotent stem cell (iPS), by method known by a person of ordinary skill in the art. For example, methods to produce skin derived iPS cell derived-cardiomyocytes have been described in Mauritz et al., Circulation. 2008;118:507-517, and disclosed in International Application WO2008/088882 which is incorporated herein by reference. In some embodiments, an iPS cell used to derive a CVP cells can be produced by any method known in the art can be used, for example virally-induced or chemically induced generation of iPS cells as disclose in EP1970446, US2009/0047263, US2009/0068742, and 2009/0227032, which are incorporated herein in their entirety by reference.

The term “induced pluripotent stem cell” (or “iPS cell”), as used herein, refers to a pluripotent stem cell induced from a somatic cell, e.g., a differentiated somatic cell. iPS cells are capable of self-renewal and differentiation into cell fate-committed stem cells, including neural stem cells, as well as various types of mature cells.

Non-cardiomyocyte cells that are suitable for generating iPS-derived CVP cells for use in the methods and tissue engineered myocardium as disclosed herein include stem cells, progenitor cells, and somatic cells. Suitable cells include, but are not limited to, embryonic stem cells; adult stem cells; induced pluripotent stem (iPS) cells; skin fibroblasts; skin stem cells; cardiac fibroblasts; bone marrow-derived cells; skeletal myoblasts; neural crest cells; and the like. In some embodiments, a iPS cell for use in generating a iPS-derived CVP cell is derived from a stem cell, a non-cardiomyocyte somatic cell, or a progenitor cell is a human stem cell, a human non-cardiomyocyte somatic cell, or human progenitor cell. In other embodiments, a iPS cell for use in generating a iPS-derived CVP cell derived from a stem cell, non-cardiomyocyte somatic cell, or progenitor cell is a non-human primate stem cell, a non-human primate non-cardiomyocyte somatic cell, or non-human primate progenitor cell. In other embodiments, a iPS cell for use in generating a iPS-derived CVP cell is derived from a stem cell, non-cardiomyocyte somatic cell, or progenitor cell is a rodent stem cell, a rodent non-cardiomyocyte somatic cell, or a rodent progenitor cell. In some embodiments, a iPS cell for use in generating a iPS-derived CVP cell is derived from a stem cells, non-cardiomyocyte somatic cells, and progenitor cells from other mammals (e.g., ungulate cells, e.g., porcine cells) are also contemplated.

In some embodiments, a CVP cell is derived from an induced pluripotent stem (iPS) cell. iPS cells are generated from somatic cells, including skin fibroblasts, using, e.g., known methods. iPS cells produce and express on their cell surface one or more of the following cell surface antigens: SSEA-3, SSEA-4, TRA-1-60, TRA-1-81 , TRA-2-49/6E, and Nanog. In some embodiments, iPS cells produce and express on their cell surface SSEA-3, SSEA-4, TRA-1-60, TRA-1-81, TRA-2-49/6E, and Nanog. iPS cells express one or more of the following genes: Oct-3/4, Sox2, Nanog, GDF3, REX1, FGF4, ESG1, DPP A2, DPPA4, and hTERT. In some embodiments, an iPS cell expresses Oct-3/4, Sox2, Nanog, GDF3, REX1, FGF4, ESG1, DPPA2, DPPA4, and hTERT. Methods of generating iPS are known in the art, and any such method can be used to generate iPS. See, e.g., Takahashi and Yamanaka (2006) Cell 126:663-676; Yamanaka et. al. (2007) Nature 448:313-7; Wernig et. al. (2007) Nature 448:318-24; Maherali (2007) Cell Stem Cell 1 :55-70.

iPS cells can be generated from somatic cells (e.g., skin fibroblasts) by genetically modifying the somatic cells with one or more expression constructs encoding Oct-3/4 and Sox2. In some embodiments, somatic cells are genetically modified with one or more expression constructs comprising nucleotide sequences encoding Oct-3/4, Sox2, c-myc, and Klf4. In some embodiments, somatic cells are genetically modified with one or more expression constructs comprising nucleotide sequences encoding Oct-4, Sox2, Nanog, and LIN28.

### Engineering Scaffold and Free-Standing Polymer Structure

As disclosed herein, one aspect of the present invention relates to the use of the CVPs in combination with engineered substrates and scaffolds for controlled differentiation of the CVPs into mature ventricular cardiomyocytes resulting in the generation of functional cardiac tissue.

In some embodiments, the scaffold used to generate the MTF tissue as disclosed herein is patterned, for example the scaffold is engineered so that the cellular environment at multiple spatial scales (nanometer to meter) is modified in order to direct progenitor cells down specific differentiation pathways and to subsequently organize the CVP cells into two-dimensional (2D) and three-dimensional (3D) myocardial tissue structures. In some embodiments, the scaffold is a free-standing polymer structure which is specially organized from the nanometer to centimeter length. In a preferred embodiments, and can be a free-standing polymer as disclosed in International Patent Application WO2008/045506 which is incorporated in its entirety herein by reference.

Accordingly, the present invention provides an improved tissue-engineered myocardium composition comprising a free-standing polymer structure and CVP cells. One advantage of the integration of these CVP cells into an engineered scaffold such as the free-standing polymer structure as disclosed herein is that the free-standing polymer structure provides environmental cues to control and direct the differentiation of CVP cells into ventricular cardiomyocytes to generate a functional contracting tissue engineered myocardium structure. The free-standing polymer structure is engineered from the nanometer to micrometer to millimeter to macroscopic length cells, and comprises factors such as, but are not limited to, material mechanical properties, material solubility, spatial patterning of bioactive compounds, spatial patterning of topological features, soluble bioactive compounds, mechanical perturbation (cyclical or static strain, stress, shear, etc . . . ), electrical stimulation, and thermal perturbation.

As disclosed herein, a freestanding functional tissue structure for use in the generation of the tissue engineered myocardium as disclosed herein can contain a flexible polymer scaffold (e.g., biologically derived) that is imprinted with a predetermined pattern and CVP cells attached to said polymer. The CVP cells are spatially organized according to the imprinted pattern, and the CVP cells can differentiate into ventricular cardiomyocytes which are functionally active. By functionally active, it is meant that the cell attached to the polymer scaffold comprises at least one function of that cell type in its native environment. For example, a cardiomyocyte cell contracts, e.g., a cardiomyocyte cell contracts along a single axis. The tissue engineered myocardium composition can optionally contain a plurality of scaffolds or films. The construction of the tissue engineered myocardium composition can be carried out by assembling the scaffolds and then seeding with CVP cells. Alternatively, the tissue engineered myocardium composition can be assembled in an iterative manner in which a scaffold is made, seeded with CVP cells, and stacked with another scaffold, which in turn is seeded with CVP cells. This seed/stack process is repeated to construct the structure. In some embodiments, any number of scaffolds coated with CVP can be stacked, for example at least 2, or at least 3, or at least 4, or a least 5, or at least 6 or a least 7 or more scaffolds coated with CVPs can be stacked. In some embodiments, the scaffold which is coated with CVP cells can be in any geometric conformation, for example, a flat sheet, a spiral, a cone, a v-like structure and the like. In some embodiments, after a culturing the CVPs on the scaffold, the scaffold is removed (e.g. bioabsorbed or physically removed), and the layers of CVP cells maintain substantially the same conformation as the scaffold, such that, for example, if the scaffold was spiral shaped, the CVPs form a 2D- and 3D-engineered myocardium tissue which is spiral shaped. In some embodiments, the shape of the scaffold is V, such that the 3D engineered myocadium is in a V-like shape such that when contraction occurs it forms a pincher like action.

In some cases a second cell types other than CVP cells can be seeded together or sequentially, e.g., for construction of muscle tissue with blood vessels where a layer of a scaffold is seeded with CVP cells and then a layer of scaffold is seeded with a different population of cells which make up blood vessels, neural tissue, cartilage, tendons, ligaments and the like. The predetermined pattern upon which CVP cells, and the combination of use of CVP cells with other populations of cells depends upon the desired functionality of the myocardial tissue. For example, ventricular myocardium with a pacemaker functionality will comprise CVP cells in combination with a pacemaker cell type, and a ventricular myocardium with ligament or tedon structures will comprise CVP cells in combination with cell types which generate tendon and ligament structures. A muscle tissue structure is composed of bundles of specialized cells capable of contraction and relaxation to create movement. As an additional example, CVP cells are incorporated into the polymer scaffold. Composition and structure of the polymer scaffold contribute to directing the differentiation of the CVP cells to ventricular cardiomyocytes, which then form a functional, engineered myocardial tissue as disclosed herein.

A method for creating biopolymer structures is carried out by providing a transitional polymer on a substrate; depositing a biopolymer on the transitional polymer; shaping the biopolymer into a structure having a selected pattern on the transitional polymer (poly(N-Isopropylacrylamide); and releasing the biopolymer from the transitional polymer with the biopolymer' s structure and integrity intact. The biopolymer is selected from an extracellular matrix protein, growth factor, lipid, fatty acid, steroid, sugar and other biologically active carbohydrates, a biologically derived homopolymer, nucleic acid, hormone, enzyme, pharmaceutical composition, cell surface ligand and receptor, cytoskeletal filament, motor protein, silks, polyprotein (e.g., poly(lysine)) or a combination thereof. For example, the biopolymer is selected from the group consisting of fibronectin, vitronectin, laminin, collagen, fibrinogen, silk or silk fibroin. For example, the biopolymer component of the structure comprises a combination of two or more ECM proteins such as fibronectin, vitronectin, laminin, collagens, fibrinogen and structurally related protein (e.g. fibrin).

The deposited structure includes features with dimensions of less than 1 micrometer. The biopolymer is deposited via soft lithography. For example, the biopolymer is printed on the transitional polymer with a polydimethylsiloxane stamp. Optionally, the process includes printing multiple biopolymer structures with successive, stacked printings. For example, each biopolymer is a protein, different proteins are printed in different (e.g., successive) printings. Alternatively, the biopolymer is deposited via self assembly on the transitional polymer. Exemplary self assembly processes include assembly of collage into fibrils, assembly of actin into filaments, and assembly of DNA into double strands. In another approach, the biopolymer is deposited via vaporization of the biopolymer and deposition of the biopolymer through a mask onto the transitional polymer. For example, the biopolymer is deposited via patterned photo-cross-linking on the transitional polymer and patterned light photo-cross-links the biopolymer in the selected pattern. The method optionally includes the step of dissolving non-cross-linked biopolymer outside the selected pattern. The patterned light changes the reactivity of the biopolymer via release of a photoliable group or via a secondary photosensitive compound in the selected pattern.

The method includes a step of allowing the biopolymer to bind together via a force selected from hydrophilic, hydrophobic, ionic, covalent, Van der Waals, and hydrogen bonding or via physical entanglement. The biopolymer structure is released by applying a solvent to the transitional polymer to dissolve the transitional polymer or to change the surface energy of the transitional polymer, wherein the biopolymer structure is released into the solvent as a freestanding structure. For example, the biopolymer is released by applying a positive charge bias to the transitional polymer, by allowing the transitional polymer to undergo hydrolysis, or by subjecting the transitional polymer to enzymatic action. The biopolymer is constructed in a pattern such as a mesh or net structure. Optionally, a plurality of structures are produced, e.g., the method includes a step of stacking a plurality biopolymer structures to produce a multi-layer scaffold.

Following construction of the biopolymer structure, living CVP cells are integrated into or onto the scaffold. For example, living CVP cells are grown in the scaffold to produce three-dimensional, anisotropic myocardium. In addition to producing functional muscle tissue for human therapeutic purposes, the methods include growing the CVP living cells in the scaffold to produce the tissue engineered myocardium composition. In other applications, the CVP cells are ES-derived CVP cells or iPS-derived CVP cells, further comprising growing the CVP cells in the scaffold where the structure, composition, ECM type, growth factors and/or other cell types assist in differentiation of the CVP cells into ventricular cardiomyocytes which form functional tissue engineered myocardium composition useful as a cardiac muscle replacement tissue, or as a tool for studying ventricular muscle development or to identify agents which modify the function of cardiac muscle (e.g. to identify cardiotoxic agents).

The methods are useful to produce a free-standing biopolymer structure. Such structures are free-standing or free-floating, e.g., they do not require a support or substrate to maintain their shape or structural integrity. Shape and integrity is maintained in the absence of a support substrate. For example, a free-standing biopolymer structure is characterized as having an integral pattern of the biopolymer with repeating features with a dimension of less than 1 mm and without a supporting substrate. Exemplary structures have repeating features with a dimension of 100 nm or less. The free-standing biopolymer structure contains at least one biopolymer selected from the group consisting of extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, and combinations thereof. Alternatively or in addition, the structure comprises at least one conducting polymer selected from poly(pyrrole)s, poly(acetylene)s, poly(thiophene)s, poly(aniline)s, poly(fluorene)s, Poly(3-hexylthiophene), polynaphthalenes, poly(p-phenylene sulfide), and poly(para-phenylene vinylene)s. The freestanding biopolymer structure is contacted with a population of CVP cells and the CVP cells are seeded on the patterned biopolymer. In some cases, the free-standing biopolymer structure comprises an integral pattern of the biopolymer and molecular remnant traces of poly(N-Isopropylacrylamide).

In one configuration, the freestanding functional tissue structure includes a flexible polymer scaffold imprinted with a predetermined pattern and CVP cells attached to the polymer. In this example, the CVP cells are spatially organized according to predetermined pattern, and the CVP cells are, or have differentiated into cells such as ventricular cardiomyocytes which are functionally active.

Also within the invention is a composition containing a plurality of freestanding tissue structures, each of which contains a flexible polymer scaffold imprinted with a predetermined pattern, CVP cells attached to the polymer. The CVP cells are located in or on the structure in spatially organized manner as determined by the pattern.

Free-standing biopolymer structures include an integral pattern of the biopolymer with repeating features having a dimension of less than 1 mm (e.g., a dimension of 100 run or less) and functions as a supporting frame during tissue formation. The structure contains an integral pattern of the biopolymer having repeating features with a dimension of less than 1 mm, e.g., less than 100 nm, and embedded within a 3-dimensional gel. As described above, the structure contains at least one biopolymer selected from extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, and combinations thereof. CVP cells are seeded on the patterned biopolymer before being embedded within a gel. Optionally, the structure contains cells mixed in with a gel precursor and thus become trapped within the gel when the gel is polymerized around the patterned biopolymer. Alternatively, the cells are seeded after the patterned biopolymer is embedded within a gel. The biopolymer structure is embedded in a gel that comprises at least one biological hydrogel selected from fibrin, collagen, gelatin, elastin and other protein and/or carbohydrate derived gels or synthetic hydrogel selected from polyethylene glycol, polyvinyl alcohol, polyacrylamide, poly(N-isopropylacrylamide), poly(hydroxyethyl methacrylate) and other synthetic hydrogels, and combinations thereof.

Free-standing biopolymer structures for use in the compositions and methods to generate the tissue engineered myocardium as disclosed herein, can be spatially organized from the nanometer to centimeter length scales and can be generated via methods described herein. In this context, “biopolymer” refers to any proteins, carbohydrates, lipids, nucleic acids or combinations thereof, such as glycoproteins, glycolipids, proteolipids, etc. These biopolymers are deposited onto a transitional polymer surface using patterning techniques that allow for nanometer-to-millimeter-to-centimeter-scale spatial positioning of the deposited biopolymers. These patterning techniques include but are not limited to soft-lithography, self-assembly, vapor deposition and photolithography, each of which is further discussed, below. Once on the surface, inter-biopolymer interactions attract the biopolymers together such that they become bound together. These interactions may be hydrophilic, hydrophobic, ionic, covalent, Van der Waals, hydrogen bonding or physical entanglement depending on the specific biopolymers involved. In the appropriate solvent, dissolution or a change in the surface energy of the transitional polymer releases the patterned biopolymer structure from the surface into solution as an integral, free-standing structure. This biopolymer structure can then be used for a variety of applications, a subset of which is listed, below.

In the context of conducted proof-of-concept experiments, structures of the extracellular matrix protein (ECM), fibronectin, were fabricated into free-standing net-like (mesh) structures. Termed, “ECM Nets,” for their appearance, the fibronectin was patterned using microcontact printing onto a less-than-1-μm-thick layer of poly(N-Isopropylacrylamide) (PIPAAm) supported by a glass cover slip. The fibronectin patterned, PIPAAm coated cover slip was placed in an aqueous medium at room temperature; the aqueous medium hydrated and dissolved the PIPAAm layering, causing the release of the ECM Net into solution. Traces of the PIPAAm may remain on the ECM Net and can be detected, e.g., via mass spectrometry, to provide an indication of an ECM Net produced via this method. The micro-pattern of the ECM Net can also be detected as a mode of determining source.

The exact spatial structure of the ECM net can be changed by altering the features of the polydimethylsiloxane (PDMS) stamp used for microcontact printing and/or by printing multiple times at different angles. While substantially orthogonal net structures are principally described and illustrated herein, other patterns (e.g., fractal, radially extending and/or branching) can also be produced. The potential applications of the technology are widespread. For example, the ability to create ECM nets enable the building of three-dimensional tissue engineering scaffolds with nanometer scale (e.g., between 5 nanometers and 1 micron) spatial control by stacking two-dimensional biopolymer sheets into a three-dimensional structure. As used herein, “two-dimensional” structures include a single layer of the basic structure (e.g., scaffold), which can have a thickness of about 5 to 500 nm (e.g., 10, 25, 50, 100, 200, 300, 400, 400 or more nm); whereas “three-dimensional” structures include multiple, stacked layers of the basic structure. Integration of living cells into these biopolymer scaffolds before release, during stacking or afterward will then allow the generation of tissues with a level of spatial control that exceeds current gel, random mesh and sponge structures used. A detailed listing of materials, methods and many potential applications are listed below.

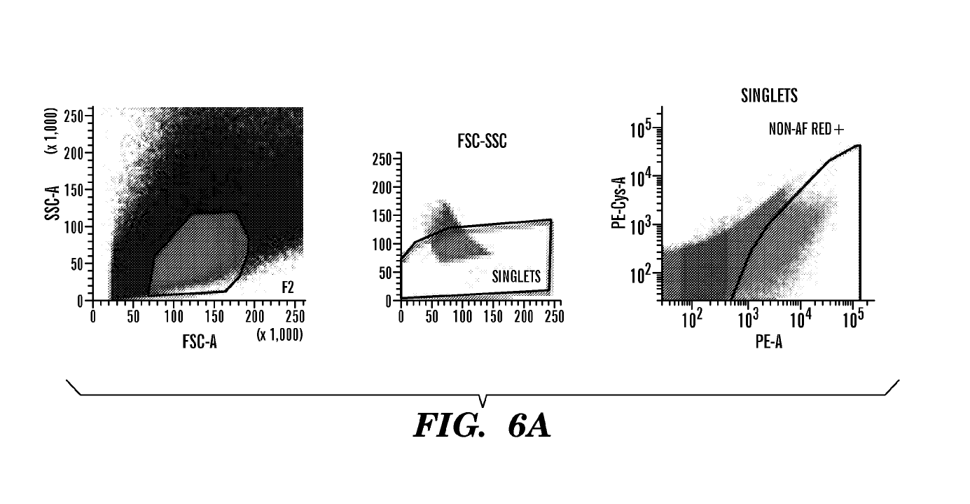

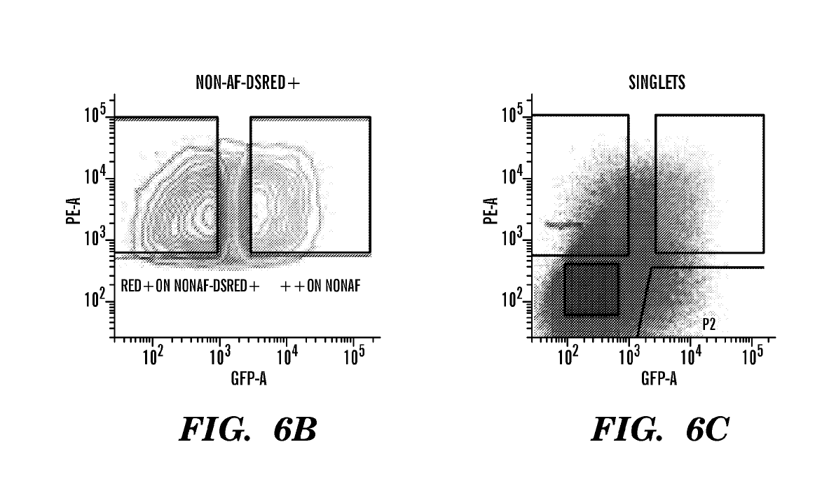

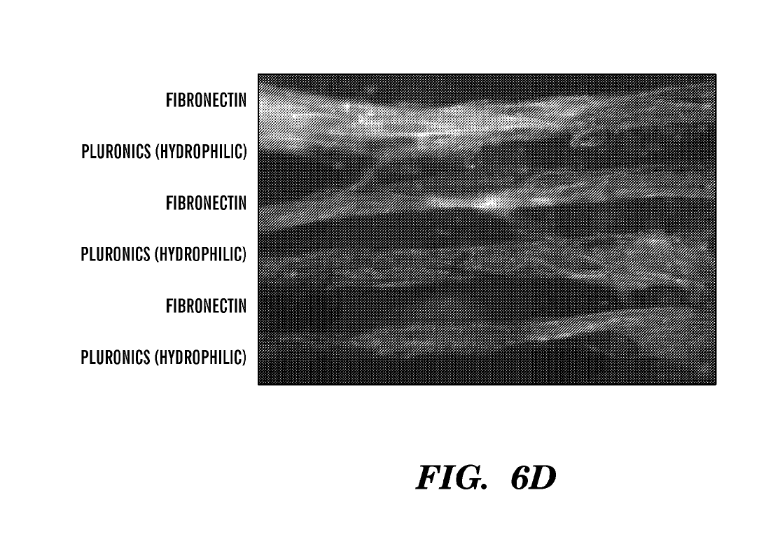

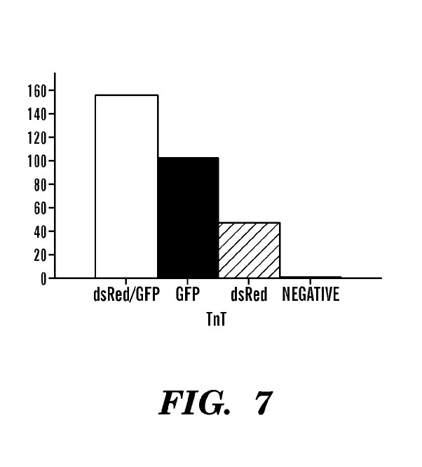

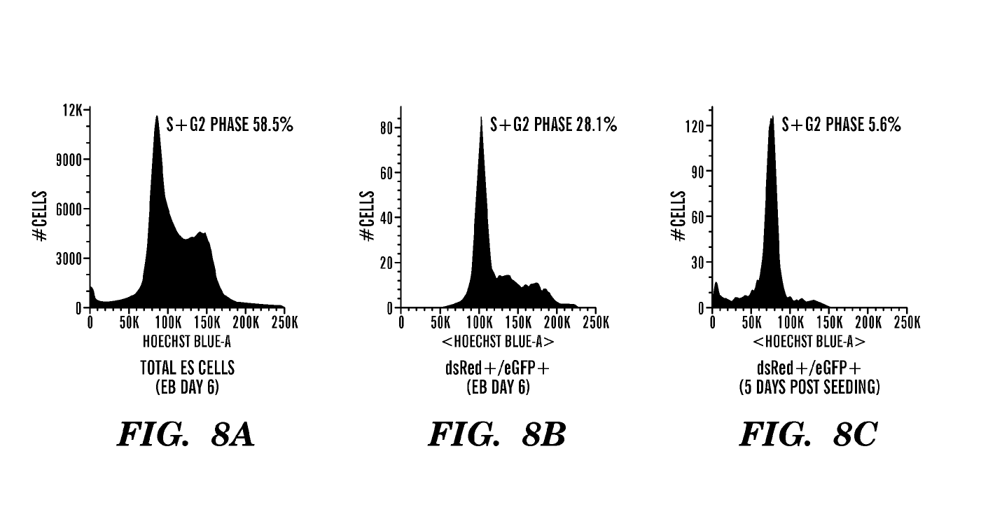

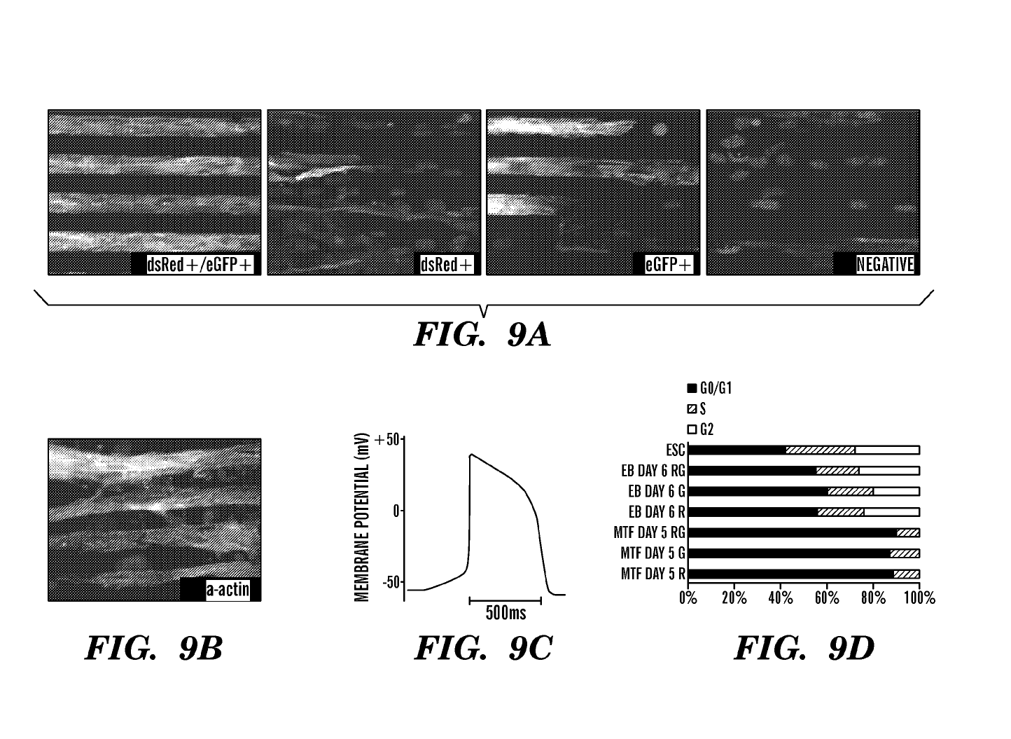

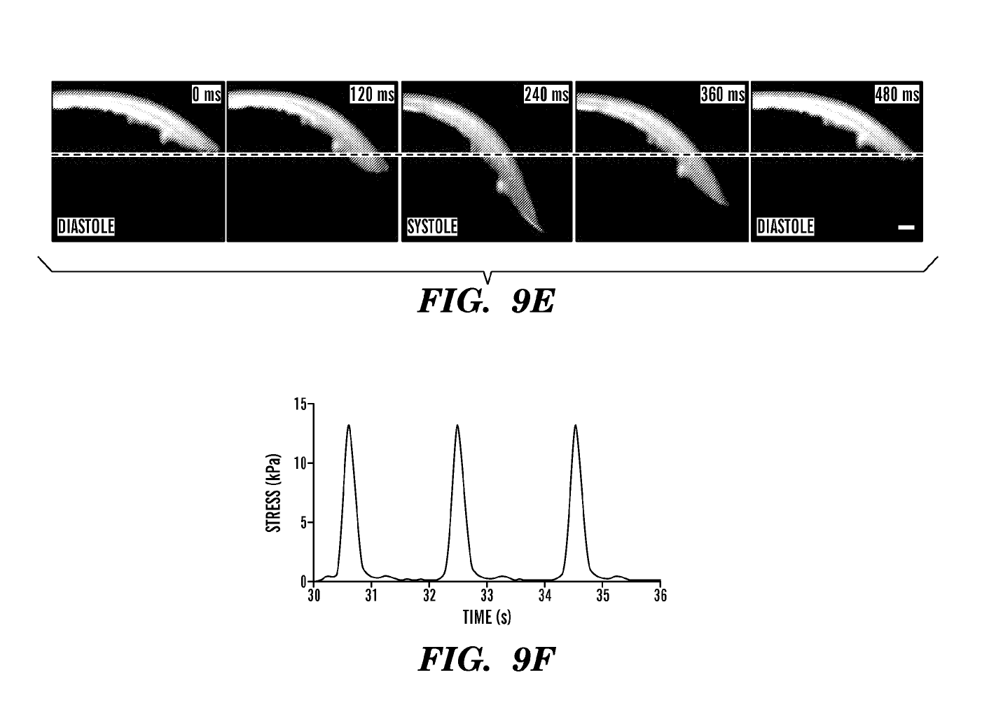

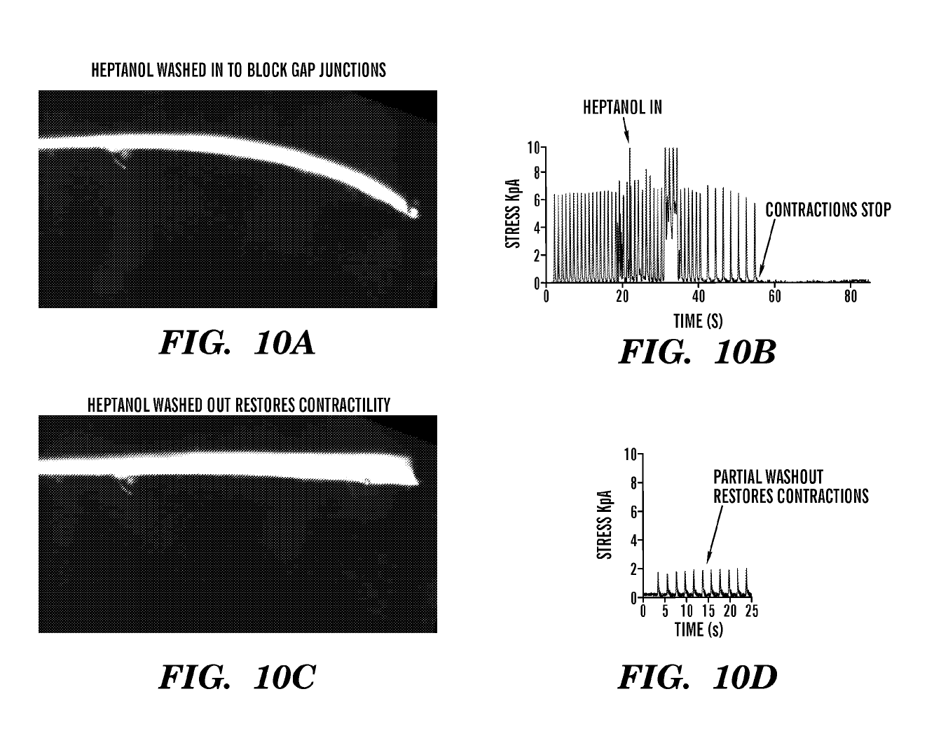

As shown in FIG. 6D and FIG. 9A, when the CVP cells are grown on a printed patterned polymer scaffold herein, such as a fibronectin patterned, PIPAAm coated polymer scaffold, the CVP cells form organized myocardial fibrils (in a uni-axial organization) on the fibronectin but not on the pluronic patterned area. In some embodiments, CVP cells are spatially organized in an anisotropic (e.g. direction-related) tissue structure, therefore to facilitate efficient electrical and mechanical activity of the MTF. Another way to organize the anisotropic tissue structure of the MTF is disclosed in Pjnappels et al., Cir. Res., 2008, 103, 167-176, which is incorporated herein in its entirity by reference.

The term “substrate” should be understood in this connection to mean any suitable carrier material to which the cells are able to attach themselves or adhere in order to form the corresponding cell composite, e.g. the tissue engineered myocardium composition as disclosed herein, such as the MTF tissue. In some embodiments, the matrix or carrier material, respectively, is present already in a three-dimensional form desired for later application. For example, bovine pericardial tissue is used as matrix which is crosslinked with collagen, decellularized and photofixed.

For example, a substrate (also referred to as a “biocompatible substrate”) is a material that is suitable for implantation into a subject onto which a cell population can be deposited. A biocompatible substrate does not cause toxic or injurious effects once implanted in the subject. In one embodiment, the biocompatible substrate is a polymer with a surface that can be shaped into the desired structure that requires repairing or replacing. The polymer can also be shaped into a part of a structure that requires repairing or replacing. The biocompatible substrate provides the supportive framework that allows cells to attach to it, and grow on it. Cultured populations of cells can then be grown on the biocompatible substrate, which provides the appropriate interstitial distances required for cell-cell interaction.

Materials for the Free-Standing Polymer Structure for Use in the Tissue Engineered Myocardium Composition.

The free-standing rigid substrate can be any rigid or semi-rigid material, selected from, e.g., metals, ceramics, polymers or a combination thereof. In particular embodiments, the elastic modulus of the substrate is greater than 1 MPa. Further, the substrate can be transparent, so as to facilitate observation during biopolymer scaffold release. Examples of suitable substrates include a glass cover slip, polymethylmethacrylate, polyethylene terephthalate film, silicon wafer, gold, etc.

The transitional, sacrificial polymer layer can be coated onto the substrate. In one embodiment, the transitional polymer is a thermally sensitive polymer that can be dissolved to cause the release of a biopolymer scaffold printed thereon. An example of such a polymer is linear, non-cross-linked poly(N-Isopropylacrylamide), which is a solid when dehydrated, and which is a solid at 37° C. (wherein the polymer is hydrated but relatively hydrophobic). However, when the temperature is dropped to less to 32° C. or less (where the polymer is hydrated but relatively hydrophilic), the polymer becomes a liquid, thereby releasing the biopolymer scaffold.

In another embodiment, the transitional polymer is a thermally sensitive polymer that becomes hydrophilic, thereby releasing a hydrophobic scaffold coated thereon. An example of such a polymer is cross-linked poly(N-Isopropylacrylamide), which is hydrophobic at 37° C. and which is hydrophilic at 32° C.

In yet another embodiment, the transitional polymer is an electrically actuated polymer that becomes hydrophilic upon application of an electric potential to thereby release a hydrophobic (or less hydrophilic) structure coated thereon. Examples of such a polymer include poly(pyrrole)s, which are hydrophobic when oxidized and hydrophilic when reduced. Other examples of polymers that can be electrically actuated include poly(acetylene)s, poly(thiophene)s, poly(aniline)s, poly(fluorene)s, poly(3-hexylthiophene), polynaphthalenes, poly(p-phenylene sulfide), and poly(para-phenylene vinylene)s, etc.

In still another embodiment, the transitional polymer is a degradable biopolymer that can be dissolved to release a structure coated thereon. In one example, the polymer (e.g., polylactic acid, polyglycolic acid, poly(lactic-glycolic) acid copolymers, nylons, etc.) undergoes time-dependent degradation by hydrolysis. In another example, the polymer undergoes time-dependent degradation by enzymatic action (e.g., fibrin degradation by plasmin, collagen degradation by collagenase, fibronectin degradation by matrix metalloproteinases, etc.). Finally, a spatially engineered surface chemistry is produced on the transitional polymer layer. The surface chemistry can be selected from the following group: (a) extracellular matrix proteins to direct cell adhesion and function (e.g., collagen, fibronectin, laminin, etc.); (b) growth factors to direct cell function specific to cell type (e.g., nerve growth factor, bone morphogenic proteins, vascular endothelial growth factor, etc.); (c) lipids, fatty acids and steroids (e.g., glycerides, non-glycerides, saturated and unsaturated fatty acids, cholesterol, corticosteroids, sex steroids, etc.);(d) sugars and other biologically active carbohydrates (e.g., monosaccharides, oligosaccharides, sucrose, glucose, glycogen, etc.); (e) combinations of carbohydrates, lipids and/or proteins, such as proteoglycans (protein cores with attached side chains of chondroitin sulfate, dermatan sulfate, heparin, heparan sulfate, and/or keratan sulfate); glycoproteins [e.g., selectins, immunoglobulins, hormones such as human chorionic gonadotropin, Alpha-fetoprotein and Erythropoietin (EPO), etc.]; proteolipids (e.g., N-myristoylated, palmitoylated and prenylated proteins); and glycolipids (e.g., glycoglycerolipids, glycosphingolipids, glycophosphatidylinositols, etc.); (f) biologically derived homopolymers, such as polylactic and polyglycolic acids and poly-L-lysine; (g) nucleic acids (e.g., DNA, RNA, etc.); (h) hormones (e.g., anabolic steroids, sex hormones, insulin, angiotensin, etc.); (i) enzymes (types: oxidoreductases, transferases, hydrolases, lyases, isomerases, ligases; examples: trypsin, collegenases, matrix metallproteinases, etc.); (j) pharmaceuticals (e.g., beta blockers, vasodilators, vasoconstrictors, pain relievers, gene therapy, viral vectors, anti-inflammatories, etc.); (k) cell surface ligands and receptors (e.g., integrins, selectins, cadherins, etc.); and (l) cytoskeletal filaments and/or motor proteins (e.g., intermediate filaments, microtubules, actin filaments, dynein, kinesin, myosin, etc.).

Methods for Generating a Free-Standing Polymer Structure for Use in the Tissue Engineered Myocardium Composition.

1) Patterning

The rigid substrate can be coated with a thin layer of the transitional polymer by a variety of methods, including spin coating, dip casting, spraying, etc. A biopolymer is then patterned onto the transitional polymer with spatial control spanning the nanometer-to-micrometer-to-millimeter-to-centimeter-length scales. This level of spatial control can be achieved via patterning techniques including but not limited to soft lithography, self assembly, vapor deposition and photolithography. Each of these techniques is discussed, in turn, below.

a) Soft Lithography: In soft lithography, structures (particularly those with features measured on the scale of 1 nm to 1 μm) are fabricated or replicated using elastomeric stamps, molds, and conformable photomasks. One such soft lithography method is microcontact printing using a polydimethylsiloxane stamp. Microcontact printing has been realized with fibronectin, laminin, vitronectin and fibrinogen and can be extended to other extracellular matrix proteins including, but not limited to collagens, fibrin, etc. Other biopolymers can be used as well, as this soft lithography method is quite versatile. There are few, if any, limitations on the geometry of the biopolymer structure(s) beyond the types of patterns that can be created in the polydimethylsiloxane stamps used for microcontact printing. The range of patterns in the stamps, in turn, is presently limited only by the current microprocessing technology used in the manufacture of integrated circuits. As such, available designs encompass nearly anything that can be drafted in modern computer-aided-design software. Multiple layers of biopolymers can be printed on top of one another using the same or different stamps with the same or different proteins to form an integrated poly-protein (poly-biopolymer) layer that can subsequently be released and used.

b) Self Assembly: Various biopolymers will spontaneously form self-assembled structures. Examples, without limitation, of self assembly include assembly of collagen into fibrils, assembly of actin into filaments and assembly of DNA into double strands and other structures depending on base-pair sequence. The self assembly can be directed to occur on the transitional layer to create a nanometer-to-millimeter-centimeter-scale spatially organized biopolymer layer. Further, self assembly can be combined with soft lithography to create a self-assembled layer on top of a soft lithographically patterned biopolymer; alternatively, the processes can be carried out in the reverse order. The self-assembled biopolymer, depending on the strength and stability of intermolecular forces, may or may not be stabilized using a cross-linking agent (for example, glutaraldehyde, formaldehyde, paraformaldehyde, etc.) to maintain integrity of the biopolymer layer upon release from the transitional layer. Otherwise, existing intermolecular forces from covalent bonds, ionic bonds, Van der Waals interactions, hydrogen binding, hydrophobic/hydrophilic interactions, etc., may be strong enough to hold the biopolymer scaffold together.

c) Vapor Deposition: Using a solid mask to selectively control access to the surface of the transitional polymer, biopolymers can be deposited in the accessible regions via condensation from a vapor phase. To drive biopolymers into a vapor phase, the deposition is performed in a controlled environmental chamber where the pressure can be decreased and the temperature increased such that the vapor pressure of the biopolymer approaches the pressure in the environmental chamber. Biopolymer surfaces produced via vapor deposition can be combined with biopolymer surfaces created by self-assembly and/or by soft lithography.

d) Patterned Photo-Cross-linking: Patterned light, x-rays, electrons or other electromagnetic radiation can be passed through a mask by photolithography; alternatively, the radiation can be applied in the form of a focused beam, as in stereolithography or e-beam lithography, to control where the transitional polymer biopolymers attach. Photolithography can be used with biopolymers that intrinsically photo-cross-link or that change reactivity via the release of a photoliable group or via a secondary photosensitive compound to promote cross-linking or breaking of the polymer chains so that the surface areas that are exposed to light are rendered either soluble or insoluble to a developing solution that is then applied to the exposed biopolymer to either leave only the desired pattern or remove only the desired pattern. The biopolymer is provided in an aqueous solution of biopolymer intrinsically photosensitive or containing an additional photosensitive compound(s).

Examples of photo-cross-linking process that can be utilized include (a) ultra-violet photo-cross-linking of proteins to RNA [as described in A. Paleologue, et al., “Photo-Induced Protein Cross-Linking to 5S RNA and 28-5.8S RNA within Rat-Liver 60S Ribosomal Subunits,” Eur. J. Biochem. 149, 525-529 (1985)]; (b) protein photo-cross-linking in mammalian cells by site-specific incorporation of a photoreactive amino acid [as described in N. Hino, et al., “Protein Photo-Cross-Linking in Mammalian Cells by Site-Specific Incorporation of a Photoreactive Amino Acid,” Nature Methods 2, 201-206 (2005)]; (c) use of ruthenium bipyridyls or palladium porphyrins as photo-activatable crosslinking agents for proteins [as described in U.S. Pat. No. 6,613,582 (Kodadek et al.)]; and (d) photocrosslinking of heparin to bound proteins via the cross-linking reagent, 2-(4-azidophenylamino)-4-(1-ammonio-4-azabicyclo[2,2,2]oct-1-yl)-6-morpho-lino-1,3,5-triazine chloride [as described in Y. Suda, et al., “Novel Photo Affinity Cross-Linking Resin for the Isolation of Heparin Binding Proteins,” Journal of Bioactive and Compatible Polymers 15, 468-477 (2000)].

2) Biopolymer Release and Scaffold Formation

The transitional polymer layer dissolves or switches states to release the biopolymer structure(s). For example, a transitional polymer layer formed of PIPAAm (non-cross-linked) will dissolve in an aqueous media at a temperature less than 32° C. In another example, a transitional polymer layer is formed of PIPAAm (cross-linked) will switch from a hydrophobic to hydrophilic state in an aqueous media at a temperature less than 32° C. The hydrophilic state will release the biopolymers. In yet another embodiment, the transitional polymer layer includes a conducting polymer, such as polypyrrole, that can be switched from a hydrophobic to hydrophilic state by applying a positive bias that switches the conducting polymer from a reduced to oxidized state. In additional embodiments, the transitional polymer layer can include a degradable polymer and/or biopolymer that undergoes time-dependent degradation by hydrolysis (as is the case, for example, for polylactic and polyglycolic acid) or by enzymatic action (for example, fibrin degradation by plasmin). These biopolymer structure(s) can then be further manipulated for the desired application.

For example, two-dimensional biopolymer scaffolds can be stacked to form a three-dimensional structure. In another example, the two-dimensional biopolymer scaffolds are seeded with cells before or after release from the transitional polymer before or after stacking to produce a three-dimensional structure.

In some embodiments, the tissue engineered myocardium as disclosed herein can comprise two-dimensional biopolymer sheets fabricated with nanometer spatial control which can be stacked to build a three-dimensional tissue-engineering scaffold. Integration of the CVP cells into these biopolymer scaffolds enables the generation of a tissue engineered myocardium as disclosed herein with a level of spatial control that extends from the micrometer scale to the meter scale (e.g., between 1 μm and 1 m) and that exceeds the spatial control provided in the generation of existing tissue engineered cardiac tissue using gel, random mesh and sponge scaffold structures or other structured scaffolds.

Use of the tissue engineered myocardium as disclosed herein using the two-dimensional biopolymer scaffold has numerous applications and utilities, including a wide array of tissue-engineering applications. Examples of products and procedures that can be produced with the scaffolds include the following: (a) three-dimensional, anisotropic myocardium used to repair infarcts, birth defects, trauma and for bench top drug testing; (b) or repair of any muscle tissue.

In another application, two-dimensional scaffolds are wrapped around a three-dimensional object to create patterned surfaces that have nanometer-to-millimeter-to-centimeter-scale features and that cannot be patterned directly using any other technique. In another embodiment, the scaffolds can be used as microstructured wound dressings (after cutting the scaffold into a size and shape to fit the wound) for repair of heart tissue, that can control the growth direction and morphology of CVP cells into ventricular cardiomyocytes in an organization on the ECM proteins in a linear and parallel orientation, for example, where the CVP cells differentiate into ventricular cardiomyocytes to maintain myocyte uni-axial alignment in the re-growth of cardiac muscle such as ventricular myocardium tissue.

In some embodiments, in addition to the CVP cells, the two-dimensional biopolymer scaffolds for use in the tissue engineered myocardium as disclosed herein can also be seeded with functional elements, such as drugs, coagulants, anti-coagulants, etc., and can be kept, e.g., in a medic's field pack. In another embodiment, the scaffold can be seeded with spray-dried cellular forms, as described in PCT/US2006/031580; which is incorporated herein by reference in its entirety. In another embodiment, the scaffold can be seeded with CVP cells where the scaffold composition and structure directs (with or without other environmental factors) directs their differentiation into ventricular cardiomyocytes. This includes any type of cardiotrophic growth factor as disclosed herein. Accordingly, in the scaffold, structure, composition, ECM type, growth factors and/or other cardiotrophic factors which assist in directing differentiation of CVP cells into ventricular cardiomyocytes can be used to aid in the production of a functional, tissue engineered myocardium as disclosed herein.

In another embodiment, the biopolymer scaffold can be embedded within a gel material to provide spatially patterned chemical, topographical and/or mechanical cues to cells. The biopolymer scaffold is constructed, as has been described, as either a single layer, or as a stacked, 3-D layered structure. A liquid, gel-precursor is then poured around the biopolymer scaffold, and then polymerized (e.g., cross-linked) into a gel. In such a case, CVP cells can either be seeded onto the biopolymer scaffold before embedding in the gel, mixed in with the gel-precursor solution before pouring around the biopolymer scaffold and crosslinking, or seeded onto the combined construct of the biopolymer scaffold embedded in the gel. Examples of gels that can be used include but are not limited to biological gels such as fibrin, collagen, gelatin, etc. and synthetic polymer hydrogels such as polyethylene glycol, polyacrylamide, etc. For example, a nerve graft can be tissue engineered by generating a biopolymer scaffold consisting of a parallel array of long fibronectin strands (such as 20 micrometers wide, 1 centimeter long), seeding CVPs on the fibronectin strands, culturing the CVP cells so they can adhere and grow along the fibronectin, embed the fibronectin and CVPs with a fibrin gel, and then place the fibrin gel with embedded fibronectin and CVPs as a therapeutic device, for example as a patch for cardiac infarction or after myocardial infarction.

In some embodiments, the scaffold is patterened with alternating surfaces, for e.g. as disclosed in the Examples, CVPs are seeded on a scaffold coated with a fibronectin and a surfactant which blocks cell adhesion (such as e.g. Pluronic F127). In some embodiments, the strips are about 20 μm wide, however strip diameters can vary, for example at least 5 μm, or at least about 10 μm, or at least about 20 μm, or at least about 30 μm, or at least about 40 μm, or at least about 50 μm or more than 50 μm. In some embodiments, the diameter of the strips coated with different surfactants (e.g. fibronectin or a surfactant which blocks cell adhesion) may vary, between the same surfactants and between different surfactants, and can be any diameter from 1 μm-50 μm or greater than 50 μm.

An additional embodiment is the fabrication of fabrics. For example, the biopolymer scaffold is built using silk, the strongest biological fiber known to man. The ability to control silk alignment at the nano/micro scale will result in fabrics with unique strength and other physical properties such as the ability to create engineered spider webs. Such engineered spider webs could be used for a multitude of applications such as, but not limited to, catching clots in the blood stream, removing (filtering) particulates from gases or fluids and ultra-light, ultra- strong fabrics for high-performance activities providing abrasion resistance, perspiration wicking and other properties.

In another embodiment, the tissue engineered myocardium composition comprises a two-dimensional biopolymer scaffold seeded with a population of CVP cells and at least one other population of cells. By way of a non-limiting example, a tissue engineered myocardium composition as disclosed herein can comprise a two-dimensional biopolymer scaffold seeded with a population of CVP cells and a cell population which functions as a biological pacemaker, and/or a cell population which forms a functional structure, such as cells which form a ligament and/or tendon structure. In such embodiments, the second cell population can be mixed with the CVP cell population, or alternatively, the CVP cell population is separated spatially from the other population(s) of cells.

Other Scaffolds and Variants Thereof

In some embodiments, the substrate useful in the methods and compositions as disclosed herein can be any biocompatible substrate. In some embodiments, the substrate is bioresorbable and/or biodegradable. Further, in some embodiments the substrate is biocompatible and bioreplacable.

In some embodiments, a scaffold useful in the methods as disclosed herein is a decellularized tissue sheet, such as a decellularized pericardial tissue which is disclosed in U.S. Patent Application 2008/0195229 and International Patent Application WO/2003/050266 which are incorporated herein in their entirety by reference, or other sheet such as a perfusion-decellularized matrix as disclosed in Ott et al., 2008, Nature Medicine 14, 213-221 which is incorporated herein by reference. In another embodiment, a substrate useful in the methods and compositions as disclosed herein is a commercially available scaffold, such as INTEGRA® Dermal Regeneration Template, which is bilayer membrane system comprising a 2 layers: (1) a first layer of a porous matrix of fibers of cross-linked bovine tendon collagen and a glycosaminoglycan (chondroitin-6-sulfate) that is manufactured with a controlled porosity and defined degradation rate. A second layer (2) comprising a temporary epidermal substitute layer is made of synthetic polysiloxane polymer (silicone) and functions to control moisture loss from the wound. The first (1) layer serves as a matrix for the infiltration of fibroblasts, macrophages, lymphocytes, and capillaries derived from the wound bed. As healing progresses an endogenous collagen matrix is deposited by fibroblasts; simultaneously, this first layer of INTEGRA® Dermal Regeneration Template is degraded. Upon adequate vascularization of the dermal layer and availability of donor autograft tissue, the temporary silicone (2) layer can optionally be removed and a thin, meshed layer of epidermal autograft is placed over the “neodermis.”

In some embodiments, a scaffold useful in the methods as disclosed herein is a two-dimensional scaffold. In alternative embodiments, a scaffold useful in the methods as disclosed herein is a three-dimensional scaffold. In some embodiments, a two-dimensional scaffold is configured and spatially organized to form a three-dimensional scaffold.

In one embodiment, a bioreplaceable material for use as a scaffold in the methods and compositions as disclosed herein is submucosal tissue. In one embodiment, the submucosa tissue suitable in accordance with the invention comprises natural collagenous matrices that include highly conserved collagens, matrix proteins, glycoproteins, proteoglycans, and glycosaminoglycans in their natural configuration and natural concentrations, and other factors. In some embodiments, the submucosal tissue is from the intestine of a warm-blooded vertebrate. In some embodiments, the submucosal tissue is from the small intestine. In some embodiments, the vertebrate is a mammal. In some embodiments, the submucosal tissue is a commercially available material, such as SURGISIS® which is available from Cook Biotech Incorporated (Bloomington, Ind.).

In one embodiment the bioreplaceable material for use as a scaffold in the methods and compositions as disclosed herein comprises small intestinal submucosa of a warm blooded vertebrate. In one embodiment, the material comprises the tunica submucosa along with the lamina muscularis mucosa and the stratum compactum of a segment of intestine, said layers being delaminated from the tunica muscularis and the luminal portion of the tunica mucosa of said segment. Such a material is referred to herein as small intestinal submucosa (SIS). In accordance with one embodiment of the present invention the intestinal submucosa comprises the tunica submucosa along with basilar portions of the tunica mucosa of a segment of intestinal tissue of a warm-blooded vertebrate. While porcine SIS is widely used, it will be appreciated that intestinal submucosa can be obtained from other animal sources, including cattle, sheep, and other warm-blooded mammals.

The preparation of SIS from a segment of small intestine is disclosed in U.S. Pat. No. 4,902,508 which is incorporated herein by reference. A segment of intestine is first subjected to abrasion using a longitudinal wiping motion to remove both the outer layers (particularly the tunica serosa and the tunica muscularis) and the inner layers (the luminal portions of the tunica mucosa). Typically the SIS is rinsed with saline and optionally stored in a hydrated or dehydrated state until use. Details of the characteristics and properties of intestinal submucosa (SIS) which one can use in the methods and compositions as disclosed herein are described in U.S. Pat. No. 4,352,463, U.S. Pat. No. 4,902,508, U.S. Pat. No. 4,956,178, U.S. Pat. No. 5,281,422, U.S. Pat. No. 5,372,821, U.S. Pat. No. 5,445,833, U.S. Pat. No. 5,516,533, U.S. Pat. No. 5,573,784, U.S. Pat. No. 5,641,518, U.S. Pat. No. 5,645,860, U.S. Pat. No. 5,668,288, U.S. Pat. No. 5,695,998, U.S. Pat. No. 5,711,969, U.S. Pat. No. 5,730,933, U.S. Pat. No. 5,733,868, U.S. Pat. No. 5,753,267, U.S. Pat. No. 5,755,791, U.S. Pat. No. 5,762,966, U.S. Pat. No. 5,788,625, U.S. Pat. No. 5,866,414, U.S. Pat. No. 5,885,619, U.S. Pat. No. 5,922,028, U.S. Pat. No. 6,056,777 and WO-97/37613, which are incorporated herein in their entirety by reference. SIS, in various forms, is commercially available from Cook Biotech Incorporated (Bloomington, Ind.). In some embodiments, the submucosal tissue is a commercially available, such as SURGISIS® which is available from Cook Biotech Incorporated (Bloomington, Ind.).

In one embodiment an intestinal submucosa matrix is used as the starting material, and the material is comminuted by tearing, cutting, grinding, shearing and the like in the presence of an acidic reagent selected from the group consisting of acetic acid, citric acid, and formic acid. In one embodiment the acidic reagent is acetic acid. In one embodiment, the intestinal submucosa is ground in a frozen or freeze-dried state to prepare a comminuted form of SIS. Alternatively, comminuted SIS can also be obtained by subjecting a suspension of pieces of the submucosa to treatment in a high speed (high shear) blender, and dewatering, if necessary, by centrifuging and decanting excess water. In some embodiments, the bioreplaceable material is a material extracted from SIS, named SISH.

Preparations of the submucosa tissue compatible with the methods and compositions as described herein are described in U.S. Pat. Nos. 4,902,508; 4,956,178 and 5,281,422 and 6,893,666 the disclosures of which are expressly incorporated herein in their entirety by reference in its entirety. In some embodiments, submucosal tissue is harvested from various warm blooded vertebrate sources, for example small intestine harvested from animals raised for meat production, including but not limited to, porcine, ovine or bovine species, but not excluding other warm-blooded vertebrate species. This tissue can be used in either its natural configuration or in a comminuted or partially enzymatically digested fluid form. Vertebrate submucosa tissue is a plentiful by-product of commercial meat production operations and is thus a low cost graft material, especially when the submucosal tissue is in its native layer sheet configuration.

Suitable submucosal intestinal-derived submucosal tissue for use in the methods and compositions as disclosed herein typically comprises the tunica submucosa delaminated from both the tunica muscularis and at least the luminal portion of the tunica mucosa. In one embodiment of the present invention, the intestinal submucosa tissue comprises the tunica mucosa and a basilar portion of the tunica mucosa, which can include the lamina muscularis mucosa and the stratum compactum, which layers are known to vary in thickness and in composition definition and dependent on the vertebrate species.

In some embodiments, the preparation of the submucosa tissue for use in accordance with this invention is as described in U.S. Pat. No. 4,902,508, the disclosure of which is expressly incorporated herein in its entirety by reference. A segment of vertebrate intestine, preferably harvested from porcine, ovine or bovine species, but not excluding other species, is subjected to abrasion using a longitudinal wiping motion to remove outer layers, comprising smooth muscle tissue and the innermost layer, e.g. the luminal portion of the tunica mucosa. The submucosal tissue is rinsed with saline and optionally sterilized; it can be stored in a hydrated or dehydrated state. Lyophilized or air-dried submucosa tissue can be rehydrated optionally stretched and used in accordance with this invention without significant loss of its cell proliferation-inducing activity.

Submucosal tissue prepared from warm-blooded vertebrate organs typically has an abluminal and a luminal surface. The luminal surface is the submucosal surface facing the lumen of the organ source and is typically adjacent to the inner mucosal layer in the organ source, whereas the abluminal surface is the submucosal surface facing away from the lumen of the organ source and typically is in contact with the smooth muscle tissue of the organ source.

The submucosal tissue material of the present invention can be preconditioned by stretching the material in a longitudinal or lateral direction as described in U.S Pat. No. 5,275,826, the disclosure of which is incorporated herein it its entirety by reference.

In some embodiments, strips or pieces of the submucosa tissue can be fused together to form a unitary multi-layered submucosal tissue construct having a surface area greater than any individual strips or pieces of submucosal tissue. The process of forming a larger area/multi-layer submucosal tissue construct is described in U.S. Pat. 2002/0103542, the disclosure of which is incorporated herein in its entirety by reference. In summary, the process of forming large area sheets of a portion of submucosal tissue comprises overlapping at least a portion of another strip of submucosal tissue and applying pressure at least to the overlapped portions under condition allowing dehydration of the submucosal tissue. Under these conditions, the overlapped portions will become “fused” to form a large unitary sheet of tissue.

The large area constructs consist essentially of submucosal tissue, substantially free of potentially compromising adhesives and chemical pretreatments, and they have a greater surface area and greater mechanical strength than individual strips used to form tissue implant material. The multi-layered submucosal tissue can optionally be perforated as described in U.S. patent application Ser. No. 08/418,515, the disclosure of which is expressly incorporated herein by reference. The perforations of the submucosal tissue construct allow extracellular fluids to pass through the tissue graft material, decreasing fluid retention within the graft and enhancing the remodeling properties of the tissue grafts. The perforation of the submucosal tissue is especially beneficial for multi-laminate tissue graft constructs wherein the perforations also enhance the adhesive force between adjacent layers.

In some embodiments, the submucosal tissue useful in the methods and compositions as disclosed herein can also be in a fluidized form. Submucosal tissue can be fluidized by comminuting the tissue and optionally subjecting it to enzymatic digestion to form a substantially homogenous solution. The preparation of fluidized forms of submucosa tissue is described in U.S. Pat. No. 5,275,826, the disclosure of which is expressly incorporated herein in its entirety by reference. Fluidized forms of submucosal tissue are prepared by comminuting submucosa tissue by tearing, cutting, grinding, or shearing the harvested submucosal tissue. Thus pieces of submucosal tissue can be comminuted by shearing in a high speed blender, or by grinding the submucosa in a frozen or freeze-dried state to produce a powder that can thereafter be hydrated with water or a buffered saline solution to form a submucosal fluid of liquid, gel-like or paste-like consistency. The fluidized submucosa formulation can further be treated with enzymes such as protease, including trypsin or pepsin at an acidic pH, for a period of time sufficient to solubilize all or a major portion of the submucosal tissue components and optionally filtered to provide a homogenous solution of partially solubilized submucosa.

The graft compositions for the methods described herein can be sterilized using conventional disinfection/sterilization techniques including glutaraldehyde tanning, formaldehyde tanning at acidic pH, propylene oxide treatment, ethylene oxide treatment, gas plasma sterilization, gamma irradiation or electron beam treatment, and peracetic acid (PAA) disinfection. Sterilization techniques which do not adversely affect the mechanical strength, structure, and biotropic properties of the submucosal tissue are preferred. For instance, strong gamma irradiation can cause loss of strength of the sheets of submucosal tissue. Preferred sterilization techniques include exposing the graft to peracetic acid, 1-4 Mrads gamma irradiation (more preferably 1-2.5 Mrads of gamma irradiation) or gas plasma sterilization. Typically, the submucosal tissue is subjected to two or more sterilization processes. After the submucosal tissue is treated in an initial disinfection step, for example by treatment with peracetic acid, the tissue can be wrapped in a plastic or foil wrap and sterilized again using electron beam or gamma irradiation sterilization techniques.

As discussed above, submucosal tissue constructs applicable to the methods described herein can comprise intestinal submucosal tissue delaminated from both the tunica muscularis and at least the luminal portion of the tunica mucosa of warm-blooded vertebrate intestine, or a digest thereof. Such compositions or other implant compositions described herein can be combined with an added growth factor such as vascular endothelial growth factor, nerve growth factor or fibroblast growth factor or growth factor-containing extracts of submucosal tissue.

In one embodiment, solid forms of submucosal tissue are combined with one or more growth factors by soaking the tissue in a buffered solution containing the growth factor. For example the submucosal tissue is soaked for 7-14 days at 4° C. in a PBS buffered solution containing about 5 to about 500 mg/ml, or more preferably 25 to about 100 mg/ml of the growth factor. Submucosal tissue readily bonds to proteins and will retain an association with a bioactive agent for several days. However, to enhance the uptake of the growth factors into the submucosal tissue, the tissue can be partially dehydrated before contacting the growth factor solution. For compositions comprising fluidized, solubilized or guanidine extracts of submucosal tissue, lyophilized powder or solutions of growth factors can be directly mixed with the submucosal tissue. For example, fluidized or solubilized submucosal tissue can be mixed with a growth factor and then packed within a tube of submucosal tissue (or other biodegradable tissue). The open end of the tube can then be sealed shut after filling the tube with the fluidized or solubilized submucosal tissue.

In some embodiments, a substrate has a substantially smooth surface. In further embodiments, the substrate is mechanically strong and also malleable. In some embodiments, the substrate is malleable under non-physiological conditions, for example but not limited to by temperature above body temperature, and for example by pressures exceeding normal physiological pressures, for example, by mechanical manipulation or mechanical shaping or by an altered surrounding environment, for example excessive heat, pressure or acidic or alkali conditions. In some embodiments, the substrate is malleable under non-physiological conditions, for example, where substrate is heated to be malleable, for example heated to 50-80° C., the substrate is molded prior to seeding of the cells.

In one embodiment, the substrate is biocompatible, and biodegrades or autocatalytically degrades in vivo into biocompatible byproducts. Not to be bound by theory, but prevailing mechanism for polymer degradation is chemical hydrolysis of the hydrolytically unstable backbone of the PLGA polymers. This occurs in two phases. In the first phase, water penetrates the polymer, preferentially attacking the chemical bonds in the amorphous phase and converting long polymer chains into shorter water-soluble fragments. Because this occurs initially in the amorphous phase, there is a reduction in molecular weight without a loss in physical properties since the polymer matrix is still held together by the crystalline regions. The reduction in molecular weight is soon followed by a reduction in physical properties, as water begins to fragment the material. In the second phase, enzymatic attack and metabolization of the fragments occurs, resulting in a rapid loss of polymer mass. This type of degradation, when the rate at which water penetrates the substrate material exceeds that at which the polymer is converted into water-soluble materials (resulting in erosion throughout the substrate), is termed “bulk erosion” (Hubbell and Langer, 1995). The rate of degradation of PLGA's can be controlled, in part by the copolymer ratio with higher glycolide or lactide ratios favoring longer degradation times. Polymers of varying copolymer ratios including PLA, PLGA75:25, and PLGA50:50 have different degradation rates, with PLGA50:50 degrading the quickest, followed by PLGA 75:25 then PLA. Therefore, with increasing percentage of PGA and concurrent decrease in percentage of PLA in a co-polymer of PLGA increases the rate of degradation compared to PLA alone, and thus the rate of degradation can be tailored to the desired use. Any ration of PLA:PGA copolymer is encompassed for use in the present invention.

In some embodiments, the substrate comprises at least one of polyglycolic acid (PGA), polylactic acid (PLA), poly(lactic-co-glycolic) acid (PLGA), polyanhydride, polycapralactone (PCL), polydioxanone and polyorthoester. One of the most common polymers used as a biomaterial is the polyester copolymer poly(lactic acid-glycolic acid) (PLGA). PLGA is highly biocompatible, degrades into biocompatible monomers and has a wide range of mechanical properties making this copolymer and its homopolymers, PLA and PGA, useful in skeletal repair and regeneration. The substrate can be porous or non-porous comprising these polymers for use in bone repair have been prepared using various techniques.

The substrate of the present invention can also be a material that comprises an absorbable polymer material and other materials. In some embodiments, other materials can be selected to be used as the resorbable material, which can be selected from the group consisting of hydroxyapatite (HAP), tricalcium phosphate (TCP), tetracalcium phosphate (TTCP), dicalcium phosphate anhydrous (DCPA), dicalcium phosphate dihydrate (DCPD), octacalcium phosphate (OCP), calcium pyrophosphate (CPP), collagen, gelatin, hyaluronic acid, chitin, and poly(ethylene glycol). In alternative embodiments, the substrate can also comprise additional material, for example, but are not limited to calcium alginate, agarose, types I, II, IV or other collagen isoform, fibrin, hyaluronate derivatives or other materials (Perka C. et al. (2000) J. Biomed. Mater. Res. 49:305-311; Sechriest V F. et al. (2000) J. Biomed. Mater. Res. 49:534-541; Chu C R et al. (1995) J. Biomed. Mater. Res. 29:1147-1154; Hendrickson D A et al. (1994) Orthop. Res. 12:485-497).

In some embodiments, the substrate composed of a poly(lactic acid-co-glycolic acid) [PLGA], can be prepared as a composite with other materials. For example, other materials include for example, but not limited to calcium phosphate ceramic, for example as HA, for engineering of surface modifications of cortical bone allografts, and in some embodiments, the PLGA can be prepared in conjunction with an osteoconductive buffering agent such as HA. Such materials can also be used as fillers or bulking agents, or buffering compounds. HA is a buffering compound since it neutralizes acidic breakdown products of biodegradable polymers such as lactic acid and glycolic acid containing polymers, thereby diminishing the likelihood these materials could cause cytotoxicity, separation of the implant and sepsis.

In some embodiments, the scaffold for use in the methods and compositions as disclosed herein can additionally provide controlled release of bioactive factors to the CVP seeded cells, for example, growth factors and other agents to sustain or control subsequent cell growth and proliferation of the cells coated on the substrate of the present invention. In such a way, the CVP cells or CVP-derived cells are supplied with a constant source of growth factors and other agents for the duration of the lifetime of the cell coated scaffold. In some embodiments, the growth factors and other agents are cardiotrophic factors commonly known in the art.

In a further embodiment, instead of a protein growth factor or agent released by the scaffold on degradation, a gene or other nucleotide molecule encoding the stimulatory factor can be released. For example but not limited to, the nucleotide molecule can be DNA (double or single-stranded) or RNA (e.g. mRNA, tRNA, rRNA), or it can be an antisense nucleic acid molecule, such as antisense RNA that can function to disrupt gene expression or growth factors themselves including TGF-beta 1 and 2, and IGF-1. The nucleic acid segments can be genomic sequences, including exons or introns alone or exons and introns, or coding cDNA regions, or any nucleic acid construct, for example genes or gene fragments that one desires to transfer to a bone progenitor cells or cells coating the substrate, for example chondrocytes. Suitable nucleic acid segments can also be in virtually any form, such as naked DNA or RNA, including linear nucleic acid molecules and plasmids, or nucleic acid analogues, such as peptide nucleic acid (PNA), pseudo-complementary nucleic acid (pc-PNA), locked nucleic acid (LNA) and other agents, such as peptides, aptamers, RNAi etc, or as a functional insert within the genomes of various recombinant viruses, including viruses with DNA genomes and retroviruses.

In some embodiments, the scaffold for use in the methods and compositions as disclosed herein is coated with a solid which do not react with the scaffold. Generally, the added solids have an average diameter of less than about 1.0 mm and preferably will have an average diameter of about 50 to about 500 microns. Preferably, the solids are present in an amount such that they will constitute from about 1 to about 50 volume percent of the total volume of the particle and polymer-solvent mixture (wherein the total volume percent equals 100 volume percent). Exemplary solids include, but are not limited to, particles of demineralized bone, calcium phosphate particles, Bioglass particles, calcium sulfate, or calcium carbonate particles for bone repair, leachable solids for pore creation and particles of bioabsorbable polymers that are effective as reinforcing materials or to create pores as they are absorbed, and non-bioabsorbable materials. Suitable leachable solids include nontoxic leachable materials such as salts (e.g., sodium chloride, potassium chloride, calcium chloride, sodium tartrate, sodium citrate, and the like), biocompatible mono and disaccharides (e.g., glucose, fructose, dextrose, maltose, lactose and sucrose), polysaccharides (e.g., starch, alginate, chitosan), water soluble proteins (e.g., gelatin and agarose). The leachable materials can be removed by immersing the substrate with the leachable material in a solvent in which the particle is soluble for a sufficient amount of time to allow leaching of substantially all of the particles, but which does not detrimentally alter the substrate. In one embodiment, the solvent is water, for example distilled-deionized water. Such a process is described in U.S. Pat. No. 5,514,378, which is incorporated herein in its entirety by reference.

In some embodiments, the scaffold for use in the methods and compositions as disclosed herein can be a smooth surface which also has pores on the surface, allowing for the easy adherence and stable fixation of CVP cells in pores of the surface. Importantly, in the methods of the invention provide a scaffold with pores on the surface but not interdispersed throughout the entire substrate. In addition, at least part of the substrate can be calcified. Pores on the surface of the scaffold can be created by methods commonly known by persons skilled in the art. Representative methods include, for example, solvent evaporation, where the substrate or polymer is dissolved in a solvent. Examples of organic solvents which can be used to dissolve the substrate are well known in the art and include for example, glacial acetic acid, methylene chloride, chloroform, tetrahydrofuran, and acetone. Accurate control over pore size in the substrate is desired in order to have adherence of the cells on the surface of the substrate without their penetration into the substrate itself. In some embodiments, the desired pore size of pores on the surface of the substrate is about 150-250 μm (Hulbert et al., J. Biomed. Mat. Res. 1970 4:443).

In some embodiments, the scaffold for use in the methods and compositions as disclosed herein can also be coated with, or combined with biostatic or biocidal agents. Suitable biostatic/biocidal agents include for example, but not limited to antibiotics, povidone, sugars, mucopolysaccharides, chlorobutanol, quarternary ammonium compounds such as benzalkonium chloride, organic mercurials, parahydroxy benzoates, aromatic alcohols, halogenated phenols, sorbic acid, benzoic acid, dioxin, EDTA, BHT, BHA, TBHQ, gallate esters, NDGA, tocopherols, gum guaiac, lecithin, boric acid, citric acid, p-Hydroxy benzoic acid esters, propionates, Sulfur dioxide and sulfites, nitrates and nitrites of Potassium and Sodium, diethyl pyrocarbonate, Sodium diacetate, diphenyl, hexamethylene tetramine o-phenyl phenol, and Sodium o-phenylphenoxide, etc. When employed, biostatic/biocidal agent will typically represent from about 1 to about 25 weight percent of the substrate, calculated prior to forming the shaped material. In some embodiments, the biostatic/biocidal agents are antibiotic drugs.

In some embodiments, the scaffold for use in the methods and compositions as disclosed herein is pretreated prior to seeding with the CVP cells in order to enhance the attachment of CVP cells to the scaffold substrate. For example, prior to seeding with cells, the scaffold substrate can be treated with, for example, but not limited to, 0.1M acetic acid and incubated in polylysine, polylysine, PBS, collagen, poly-laminin and other cell adhesive substances known to persons skilled in the art.

Suitable surface active agents include the biocompatible nonionic, cationic, anionic and amphoteric surfactants and mixtures thereof. When employed, surface active agent will typically represent from about 1 to about 20 weight percent of the substrate, calculated prior to forming the shaped material. It will be understood by those skilled in the art that the foregoing list of optional substances is not intended to be exhaustive and that other materials can be admixed with substrate within the practice of the present invention.

Any of a variety of medically and/or surgically useful optional substances can be incorporated in, or associated with, the scaffold substrate either before, during, or after preparation of the tissue engineered myocardial composition as disclosed herein. Thus, for example, one or more of such substances can be introduced into the scaffold, e.g., by soaking or immersing the substrate in a solution or dispersion of the desired substance(s), by adding the substance(s) to the carrier component of the cell coated substrate or by adding the substance(s) directly to cell coated substrate. Medically/surgically useful substances include physiologically or pharmacologically active substances that act locally or systemically in the host subject.

The medically/surgically useful substances are, for example but not limited to bioactive substances which can be readily combined with the cell coated substrate of this invention and include, e.g., demineralized bone powder as described in U.S. Pat. No. 5,073,373 the contents of which are incorporated herein by reference; collagen, insoluble collagen derivatives, etc., and soluble solids and/or liquids dissolved therein; antiviricides, particularly those effective against HIV and hepatitis; antimicrobials and/or antibiotics such as erythromycin, bacitracin, neomycin, penicillin, polymycin B, tetracyclines, biomycin, chloromycetin, and streptomycins, cefazolin, ampicillin, azactam, tobramycin, clindamycin and gentamycin, etc.; biocidal/biostatic sugars such as dextran, glucose, etc.; amino acids; peptides; vitamins; inorganic elements; co-factors for protein synthesis; hormones; endocrine tissue or tissue fragments; synthesizers; enzymes such as alkaline phosphatase, collagenase, peptidases, oxidases, etc.; polymer cell scaffolds with parenchymal cells; angiogenic agents and polymeric carriers containing such agents; collagen lattices; antigenic agents; cytoskeletal agents; cartilage fragments; living cells such as chondrocytes, bone marrow cells, mesenchymal stem cells; natural extracts; genetically engineered living cells or otherwise modified living cells; expanded or cultured cells; DNA delivered by plasmid, viral vectors or other means; tissue transplants; demineralized bone powder; autogenous tissues such as blood, serum, soft tissue, bone marrow, etc.; bioadhesives; bone morphogenic proteins (BMPs); osteoinductive factor (IFO); fibronectin (FN); endothelial cell growth factor (ECGF); vascular endothelial growth factor (VEGF); cementum attachment extracts (CAE); ketanserin; human growth hormone (HGH); animal growth hormones; epidermal growth factor (EGF); interlenkins, e.g., interleukin-1 (IL-1), interleukin-2 (IL-2); human alpha thrombin; transforming growth factor (TGF-beta); insulin-like growth factors (IGF-1, IGF-2); platelet derived growth factors (PDGF); fibroblast growth factors (FGF, BFGF, etc.); periodontal ligament chemotactic factor (PDLGF); enamel matrix proteins; growth and differentiation factors (GDF); hedgehog family of proteins; protein receptor molecules; small peptides derived from growth factors above; bone promoters; cytokines; somatotropin; bone digestors; antitumor agents; cellular attractants and attachment agents; immuno-suppressants; permeation enhancers, e.g., fatty acid esters such as laureate, myristate and stearate monoesters of polyethylene glycol, enamine derivatives, alpha-keto aldehydes, etc.; and nucleic acids. The amounts of such optionally added substances can vary widely with optimum levels being readily determined in a specific case by routine experimentation.

It will be understood by those skilled in the art that the foregoing list of medically/surgically useful agents and substances is not intended to be exhaustive and that other useful substances can be admixed with substrate and/or the cell coated substrate within the practice of the present invention.

The total amount of such optionally added medically/surgically useful agents and substances will typically range from about 0 to about 95, or about 1 to about 60, or about 1 to about 40 weight percent based on the weight of the entire composition prior to compression of the composition, with optimal levels being readily determined in a specific case by routine experimentation. In some embodiments, a medically/surgically useful substance is bone morphogenic proteins.

In some embodiments, the scaffold is sterilized prior to or after the seeding the CVP cells. General sterilization methods can be used, for example, but not limited to ethylene oxide or irradiating with an electron beam, and in some embodiments, where the effect of the sterilization is toxic to the cells coated on, or to be coated on the substrate, alternative sterilization methods are sought or compensatory methods adopted, for example, additional cardiotrophic growth factors can be added to the CVP cells to reduce CVP cells from detaching from the scaffold prior to forming extracellular matrix due to the use of irradiation sterilization.

### Utility of the Tissue Engineered Myocardium Composition or CVP Cell Compostion

The CVP cell composition and/or tissue engineered myocardium composition and method of their generation as disclosed herein are useful for various research applications, treatment methods, and screening methods.

Research Applications

The CVP cell composition or tissue engineered myocardium composition as disclosed herein is useful for research applications, such as for example, but not limited to, introduction of the tissue engineered myocardium into a non-human animal model of a disease (e.g., a cardiac disease) to determine efficacy of the tissue engineered myocardium in the treatment of the disease; use of the tissue engineered myocardium in screening methods to identify candidate agents suitable for use in treating cardiac disorders; and the like. For example, a tissue engineered myocardium generated herein using a subject method can be contacted with a test agent, and the effect, if any, of the test agent on a biological activity of a CVP cell of the tissue engineered myocardium, or the function contractibility of a tissue engineered myocardium, such as a MTF can be assessed, where a test agent that has an effect on a biological activity of a CVP cell population or the contractibility of the tissue engineered myocardium is a candidate agent for treating a cardiac disorder. As another example, a tissue engineered myocardium generated using a subject method can be introduced into a non-human animal model of a cardiac disorder, and the effect of the cardiomyocyte or cardiac progenitor on ameliorating the disorder can be tested in the non-human animal model.

Screening Methods

As noted above, a CVP cell composition or tissue engineered myocardium composition as disclosed herein can be used in a screening method to identify candidate agents for treating a cardiac disorder. For example, a tissue engineered myocardium can be contacted with a test agent; and the effect, if any, of the test agent on a parameter associated with normal or abnormal tissue engineered myocardium function, such as contractibility, including frequency and force of contraction is determined. Alternatively, tissue engineered myocardium generated by a subject method can be contacted with a test agent; and the effect, if any, of the test agent on a parameter associated with normal or abnormal cardiomyocyte function is determined. Such parameters include, but are not limited to, beating; expression of a cardiomyocyte-specific marker; electric signals associated with heart beating; and the like.

Accordingly, another aspect of the present invention relates to a use of a tissue engineered myocardium as disclosed herein, in assays to identify agents which affect (e.g. increase or decrease) the contractile force and/or contractibility of the tissue engineered myocardium in the presence of the agent as compared to a control agent, or the absence of an agent. Such an assay is useful to identify an agent which has a cardiotoxic effect, such as an agent which decreases contractile force, and/or cardiomyocyte atrophy, and/or results in another dysregulation of contractibility, such as arrhythmia or abnormal contraction rate. In another embodiment, such an assay is useful to identify an agent which has a cardiotoxic effects by increasing contractile force and/or other types of dysregulation such as an increase in contraction rate and could lead to the development of cardiac muscle hypertrophy.

In another embodiment, the tissue engineered myocardium disclosed herein can be used in an assay to study a cardiovascular disease. By way of an example only, the tissue engineered myocardium can comprise genetically modified cardiomyogenic progenitors, for example cardiomyogenic progenitors carrying a mutation, polymorphism or other variant of a gene (e.g. increased or decreased expression of a heterologous gene) which can be assessed to see the effects of such a gene variant on the contractile force and contractible ability of the tissue engineered myocardium. Such a tissue engineered myocardium comprising genetically modified cardiomyogenic progenitors can also be used to identify an agent which attenuates (e.g. decreases) any dysfunction in contractibility or contraction force as a result of the genetically modified cardiomyogenic progenitors, or alternatively can be used to identify an agent which augments (e.g. increases) any dysfunction in contractibility or contraction force as a result of the genetically modified cardiomyogenic progenitors.

Another aspect of the invention relates to methods to screen for agents, for example any entity or chemicals molecule or gene product which effects (e.g. increase or decrease) the functionality of the tissue engineered myocardium as disclosed herein, such as an agent which increases or decreases the contractile force, and/or frequency of contraction and/or contractibility of the tissue engineered myocardium in the presence of the agent as compared to a control agent, or the absence of an agent. In such an embodiment, an agent which increases or decreases the contractile force, and/or frequency of contraction and/or contractibility of the tissue engineered myocardium can affect the function of a CVP, for example but not limited to, an agent which promotes differentiation, proliferation, survival, regeneration, or maintenance of a population of CVP cells, or an agent which prevent the differentiation of a CVP cell into mature ventricular cardiomyocytes, and/or inhibits or negatively affects ventricular cardiomyocyte function.

Parameters are quantifiable components of cells, particularly components that can be accurately measured, desirably in a high throughput system. A parameter can be any measurable parameter related to functional contraction of the tissue engineered myocardium (such as MTF) as disclosed herein. Such parameters include, but are not limited to, MTF bending, contractile force, peak systolic stress, frequency of contraction and the like. Other parameters include changes in characteristics and markers of the CVP cells, and/or a change in the CVP phenotype, including but not limited to changes in CVP markers, cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters related to functionality of the MTF (e.g. contraction of the MTF) provide a quantitative readout, in some instances a semi-quantitative or qualitative result will also be acceptable. Readouts can include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

As discussed, an agent which effects or modulates (e.g. increase or decrease) the functionality of the tissue engineered myocardium as disclosed herein, such as an agent which increases or decreases the contractile force, and/or frequency of contraction and/or contractibility of the tissue engineered myocardium in the presence of the agent as compared to a control agent, or the absence of an agent. Typically, a MTF which comprises CVP cells as disclosed herein has an end diastole to peak diastole and back is about 500 ms, and a systolic stress generated of ˜13 kPa at 0.5-1.0 Hz. Thus, in some embodiments, any agent which increases or decreases the end diastole to peak diastole and back by a statistically significant amount, or by at least about 10% as compared to the end to diastole to peal diastole and back in the absence of an agent, or from a reference value 500 ms, is identified to have modulated the function of the tissue engineered myocardium. If an agent increases or decreases the end diastole to peak diastole and back by at least about 10% or by at least about 15% or at least about 20% or at least about 30%, or least about 40% or at least about 50% or more than 50% as compared to a reference end diastole to peak diastole value (e.g. 500 ms) is identified to have modulated the function of the tissue myocardium.

In some embodiments, any agent which increases or decreases the systolic stress generated MTF by a statistically significant amount, or by at least about 10% as compared the systolic stress generated MTF in the absence of an agent, or from the reference value of ˜13 kPa at 0.5-1.0 Hz, is identified to have modulated the function of the tissue engineered myocardium. If an agent increases or decreases the systolic stress at 0.5Hz by at least about 10% or by at least about 15% or at least about 20% or at least about 30%, or least about 40% or at least about 50% or more than 50% as compared to a reference systolic stress (e.g. 13 kPa) is identified to have modulated the function of the tissue myocardium.

Typically, a MTF which comprises CVP cells as disclosed herein has action potential with the following characteristics; Vmax=9.4±2.8 V/ms; ADP 50=165.4±14.2 ms; ADP 90=102±19 ms; and Amp=58.8±4 mV.

In some embodiments, any agent which increases or decreases the Vmax of an action potential generated by a MTF by a statistically significant amount, or by at least about 10% as compared the Vmax of an action potential generated a MTF in the absence of an agent, or from the reference value of ˜10V/ms, is identified to have modulated the function of the tissue engineered myocardium. If an agent increases or decreases the Vmax by at least about 10% or by at least about 15% or at least about 20% or at least about 30%, or least about 40% or at least about 50% or more than 50% as compared to a reference Vmax (e.g. 10 V/ms) is identified to have modulated the function of the tissue myocardium.

In some embodiments, any agent which increases or decreases the ADP 50 of an action potential generated by a MTF by a statistically significant amount, or by at least about 10% as compared the ADP 50 of an action potential generated a MTF in the absence of an agent, or from the reference value of 165 ms, is identified to have modulated the function of the tissue engineered myocardium. If an agent increases or decreases the ADP 50 by at least about 10% or by at least about 15% or at least about 20% or at least about 30%, or least about 40% or at least about 50% or more than 50% as compared to a reference ADP 50 (e.g. 165 ms) is identified to have modulated the function of the tissue myocardium.

In some embodiments, any agent which increases or decreases the ADP 90 of an action potential generated by a MTF by a statistically significant amount, or by at least about 10% as compared the ADP 90 of an action potential generated a MTF in the absence of an agent, or from the reference value of 100 ms, is identified to have modulated the function of the tissue engineered myocardium. If an agent increases or decreases the ADP 90 by at least about 10% or by at least about 15% or at least about 20% or at least about 30%, or least about 40% or at least about 50% or more than 50% as compared to a reference ADP 90 (e.g. 100 ms) is identified to have modulated the function of the tissue myocardium.

In some embodiments, any agent which increases or decreases the amplitude (Amp) of an action potential generated by a MTF by a statistically significant amount, or by at least about 10% as compared the A amplitude (Amp) of an action potential generated a MTF in the absence of an agent, or from the reference value of 58 mV, is identified to have modulated the function of the tissue engineered myocardium. If an agent increases or decreases the amplitude (Amp) by at least about 10% or by at least about 15% or at least about 20% or at least about 30%, or least about 40% or at least about 50% or more than 50% as compared to a reference amplitude (Amp) (e.g. 58 mV) is identified to have modulated the function of the tissue myocardium.

A MTF as disclosed herein can also spontaneously beat about 20 beats /min. Thus, in some embodiments, any agent which increases or decreases the frequency of beats/min of a MTF by a statistically significant amount, or by at least about 10% as compared the frequency of beat by a MTF in the absence of an agent, or from the reference value of 20 beats/min, is identified to have modulated the function of the tissue engineered myocardium. If an agent increases or decreases the frequency of beats by at least about 10% or by at least about 15% or at least about 20% or at least about 30%, or least about 40% or at least about 50% or more than 50% as compared to a reference number of beats (e.g. 20 beats/min), the agent is identified to have modulated the function of the tissue myocardium.

In another embodiment, the methods of the invention provide a screen for agents which have cardiovascular toxicity. In some embodiments, an agent (such as a drug or compound) can be an existing agent, and in other embodiments, an agent can be new or modified agent of an existing agent (e.g. a modified drug or compound or variant thereof). In another embodiment, a tissue engineered myocardium as disclosed herein can be used for screening methods of an agent which affect a CVP cell or a CVP-derived ventricular cardiomyocyte cells, and in some embodiments, the tissue engineered myocardium comprises CVP cells, or CVP-derived ventricular cardiomyocytes which are variant CVP cell, for example but not limited to a genetic variant and/or a genetically modified CVP cell.

The tissue engineered myocardium as disclosed herein is also useful for in vitro assays and screening to detect agents that are active on CVP cells, for example, to screen for agents that affect the differentiation of CVP cells, including differentiation of CVP cells along the cardiomyocyte lineage, for example ventricular cardiomyocyte lineages. Of particular interest are screening assays for agents that are active on human CVP cells. In such embodiments, the CVP cells can be ES derived or iPS derived CVP cells.

In the use of a tissue engineered myocardium as disclosed herein for the screening methods, a tissue engineered myocardium is contacted with an agent of interest, and the effect of the agent is assessed by monitoring output parameters, such force of contraction, duration of contraction, frequency of contraction, and the like. In some embodiments, additional monitoring can be performed, such as alteration of the phenotype of the CVP cells or ventricular cardiomyocytes of the tissue engineered myocardium, including but not limited to, e.g. changes in expression of markers, cell viability, differentiation characteristics, multipotenticy capacity and the like.

In some embodiments, the tissue engineered myocardium for use in screening purposes can comprise CVP cell variants, e.g. CVP cells with a desired pathological characteristic. For example, the desired pathological characteristic can include a mutation and/or polymorphism which contribute to disease pathology, such as a cardiovascular disease as that term is defined herein. In such an embodiment, a tissue engineered myocardium comprising a CVP cell with a desired pathological characteristic can be used to screen for agents which alleviate at least one symptom of the pathology.

In alternative embodiments, a tissue engineered myocardium (e.g. a MTF) comprising a population of genetic variant CVP cells, e.g. CVP cells which endogenously, or genetically have been modified to have a particular mutation and/or polymorphism, can be used to identify agents that specifically alter the function a MTF comprising a genetic variant of the CVP cells, as compared to the effect of the agent on the function of a MTF comprising normal or control CVP cells (e.g. CVP cells without the mutation and/or polymorphism). Accordingly, a tissue engineered myocardium (e.g. a MTF) comprising a population of a genetic variant CVP cells can be used to assess the effect of an agent in defined subpopulations of people and/or CVP cells which carry modification. Therefore, the present invention enables high-throughput screening of agents for personalized medicine and/or pharmogenetics. The manner in which a tissue engineered myocardium (e.g. a MTF) comprising a population of genetic variant CVP cells responds to an agent, particularly a pharmacologic agent, including the timing of responses, is an important reflection of the physiologic state of the cell.

The agent used in the screening method using a tissue engineered myocardium (e.g. a MTF) as disclosed herein can be selected from a group of a chemical, small molecule, chemical entity, nucleic acid sequences, an action; nucleic acid analogues or protein or polypeptide or analogue of fragment thereof. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide agent or fragment thereof, can be any protein of interest, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof. An agent can contact the surface of the tissue engineered myocardium (e.g. a MTF) (e.g. contact the population of CVP cells) such as by applying the agent to a media surrounding the MTF, where it contacts the CVP cells and induces its effects. Alternatively, an agent can be intracellular within the CVP cell as a result of introduction of a nucleic acid sequence into a CVP cell and its transcription to result in the expression of a nucleic acid and/or protein agent within the CVP cell. An agent as used herein also encompasses any action and/or event or environmental stimuli that a tissue engineered myocardium (e.g. a MTF) is subjected to. As a non-limiting examples, an action can comprise any action that triggers a physiological change in the a tissue engineered myocardium (e.g. a MTF), for example but not limited to; heat-shock, ionizing irradiation, cold-shock, electrical impulse (including increase or decrease in stimuli frequency and/or stimuli intensity), mechanical stretch, hypoxic conditions, light and/or wavelength exposure, UV exposure, pressure, stretching action, increased and/or decreased oxygen exposure, exposure to reactive oxygen species (ROS), ischemic conditions, fluorescence exposure etc. Environmental stimuli also include intrinsic environmental stimuli defined below.

The exposure (e.g. contacting) of a tissue engineered myocardium (e.g. a MTF) to agent may be continuous or non-continuous. In some embodiments, where the exposure (e.g. contacting) of a tissue engineered myocardium (e.g. a MTF) to agent is a non-continuous exposure, the exposure of a MTF to one agent can be followed with the exposure to a second agent, or alternatively, by a control agent (e.g. a washing step) as disclosed herein in the Examples. In some embodiments, a tissue engineered myocardium (e.g. a MTF) can be exposed to at least one agent, or at least 2, or at least 3, or at least 4, or at least 5, or more than 5 agents at any one time, and this exposure can be continuous or non-continuous, as discussed above.

The term “agent” refers to any chemical, entity or moiety, including without limitation synthetic and naturally-occurring non-proteinaceous entities. In certain embodiments the compound of interest is a small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

In some embodiments, the agent is an agent of interest including known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like. Candidate agents also include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Also included as agents are pharmacologically active drugs, genetically active molecules, etc. Compounds of interest include, for example, chemotherapeutic agents, hormones or hormone antagonists, growth factors or recombinant growth factors and fragments and variants thereof. Exemplary of pharmaceutical agents suitable for this invention are those described in, The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

The agents include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, e.g. drug candidates.

Agents such as chemical compounds, including candidate agents or candidate drugs, can be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for effect on a tissue engineered myocardium (e.g. a MTF) by adding the agent to at least one and usually a plurality of tissue engineered myocardium (e.g. a MTF) samples. A change in a parameter (e.g. a change in a parameter to indicate a change in the contraction functionality) of the tissue engineered myocardium (e.g. a MTF) in response to the agent is measured, and the result is evaluated by comparison to a reference tissue engineered myocardium (e.g. a MTF) sample. A reference tissue engineered myocardium (e.g. a MTF) sample can be, for example but not limited to, a MTF in the absence of the same agent, or a MTF in the presence of a positive control agent, where the agent is known to have a increase or decrease on at least one parameter of the contraction functionality of the MTF). In alternative embodiments, a reference tissue engineered myocardium (such as MTF) is a negative control, e.g. where the MTF is not exposed to an agent (e.g. there is an absence of an agent), or is exposed to an agent which is known not to gave an effect on at least one parameter of the contraction functionality of the MTF).

In some embodiments, the agents can be conveniently added in solution, or readily soluble form, to the tissue engineered myocardium as disclosed herein. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over a tissue engineered myocardium (e.g. a MTF), followed by the second. In a single solution method, a bolus of the test compound is added to the volume of medium surrounding a tissue engineered myocardium (e.g. a MTF). The overall concentrations of the components of the culture medium surrounding the tissue engineered myocardium should not change significantly with the addition of the bolus, or between the two solutions in a flow through method. In some embodiments, agent formulations do not include additional components, such as preservatives, that have a significant effect on the overall formulation. Thus, preferred formulations consist essentially of a biologically active agent and a physiologically acceptable carrier, e.g. water, ethanol, DMSO, etc. However, if an agent is a liquid without a solvent, the formulation may consist essentially of the compound itself.

A plurality of assays comprising a tissue engineered myocardium (e.g. a MTF) can be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, e.g. at zero concentration or below the level of detection of the agent or at or below the concentration of agent that does not give a detectable change in the phenotype or contractibility of a tissue engineered myocardium (e.g. a MTF).

Optionally, a tissue engineered myocardium (e.g. a MTF) used in a screen as disclosed herein can comprise CVP cells which have been manipulated to express a desired gene product. Gene therapy can be used to either modify a CVP cell to replace a gene product or add a heterologous gene product, or alternatively knockdown a gene product endogenous to the CVP.

In some embodiments the genetic engineering of a CVP cell on a tissue engineered myocardium (e.g. a MTF) is done to facilitate the differentiation into ventricular cardiomyocytes, or for the regeneration of tissue, to treat disease, or to improve survival of the CVP cells, either while they are present as a component of a tissue engineered myocardium (e.g. a MTF), or following implantation of a tissue engineered myocardium (e.g. a MTF) into a subject (e.g. to prevent rejection by the recipient subject). Techniques for genetically altering and transfecting cells, including CVP cells are known by one of ordinary skill in the art.

A skilled artisan could envision a multitude of genes which would convey beneficial properties to a CVP cell which is one element of the tissue engineered myocardium (e.g. a MTF) composition as disclosed herein. Furthermore, a CVP cell could be modified to convey an indirect beneficial property, such as the survival of the CVP cells following transplantation of a tissue engineered myocardium (e.g. a MTF) into a subject (discussed in more detail below). An added gene can ultimately remain in the recipient CVP cell and all its progeny, or alternatively can remain transiently, depending on the embodiment. As a non-limiting example, a gene encoding an angiogenic factor could be transfected into CVP cells prior to seeding onto the scaffold and/or prior to generation of the tissue engineered myocardium (e.g. a MTF), or alternatively a CVP cell can be transfected with a desired gene product when it is part of the tissue engineered myocardium (e.g. a MTF) composition as disclosed herein. Use of such genes, such as genes which encode an angiogenic factor may be useful for inducing collateral blood vessel formation as the ventricular myocardium is generated, particularly if the tissue engineered myocardium is used in for transplantation purposes into a subject in need of treatment, such as a subject with a cardiovascular disease or disorder. It some situations, it may be desirable to transfect a CVP cell with more than one gene, for instance, a gene which promotes survival and/or a gene which promotes angiogenesis, and/or a gene which prevents rejection by the recipient subject following transplantation of a tissue engineered myocardium (e.g. a MTF) into a subject.

In some instances, it is desirable to have the gene product from the CVP cells present in a tissue engineered myocardium (e.g. a MTF) secreted. In such cases, a nucleic acid which encodes the protein preferably contains a secretory signal sequence that facilitates secretion of the protein. For example, if the desired gene product is an angiogenic protein, a skilled artisan could either select an angiogenic protein with a native signal sequence, e.g. VEGF, or can modify the gene product to contain such a sequence using routine genetic manipulation (See Nabel et al., 1993).

The desired gene for use in modification of a CVP cell for use in the tissue engineered myocardium (e.g. a MTF) as disclosed herein can be transfected into the cell using a variety of techniques. Preferably, the gene is transfected into the cell using an expression vector. Suitable expression vectors include plasmid vectors (such as those available from Stratagene, Madison Wis.), viral vectors (such as replication defective retroviral vectors, herpes virus, adenovirus, adeno-virus associated virus, and lentivirus), and non-viral vectors (such as liposomes or receptor ligands).

A desired gene is usually operably linked to its own promoter or to a foreign promoter which, in either case, mediates transcription of the gene product. Promoters are chosen based on their ability to drive expression in restricted or in general tissue types, for example in mesenchymal cells, or on the level of expression they promote, or how they respond to added chemicals, drugs or hormones. Other genetic regulatory sequences that alter expression of a gene may be co-transfected. In some embodiments, the host cell DNA may provide the promoter and/or additional regulatory sequences. Other elements that can enhance expression can also be included such as an enhancer or a system that results in high levels of expression.

Methods of targeting genes in mammalian cells are well known to those of skill in the art (U.S. Pat. Nos. 5,830,698; 5,789,215; 5,721,367 and 5,612,205). By “targeting genes” it is meant that the entire or a portion of a gene residing in the chromosome of a cell is replaced by a heterologous nucleotide fragment. The fragment may contain primarily the targeted gene sequence with specific mutations to the gene or may contain a second gene. The second gene may be operably linked to a promoter or may be dependent for transcription on a promoter contained within the genome of the cell. In a preferred embodiment, the second gene confers resistance to a compound that is toxic to cells lacking the gene. Such genes are typically referred to as antibiotic-resistance genes. Cells containing the gene may then be selected for by culturing the cells in the presence of the toxic compound.

Methods of gene targeting in mammals are commonly used in transgenic “knockout” mice (U.S. Pat. Nos. 5,616,491; 5,614,396). These techniques take advantage of the ability of mouse embryonic stem cells to promote homologous recombination, an event that is rare in differentiated mammalian cells. Recent advances in human embryonic stem cell culture may provide a needed component to applying the technology to human systems (Thomson; 1998). Furthermore, the methods of the present invention can be used to isolate and enrich for stem cells or progenitor cells that are capable of homologous recombination and, therefore, subject to gene targeting technology. Indeed, the ability to isolate and grow somatic stem cells and progenitor cells has been viewed as impeding progress in human gene targeting (Yanez & Porter, 1998).

Treatment Methods

In another embodiment, the tissue engineered myocardium as disclosed herein can be used for prophylactic and therapeutic treatment of a cardiovascular condition or disease. By way of an example only, in such an embodiment, a tissue engineered myocardium as disclosed herein can be administered to a subject, such as a human subject by way of transplantation, where the subject is in need of such treatment, for example, the subject has, or has an increased risk of developing a cardiovascular condition or disorder.

In some embodiments, the CVP cell composition or tissue engineered myocardium composition as disclosed herein can be introduced into a subject in need thereof, e.g., a CVP cell composition or tissue engineered myocardium composition as disclosed herein can be introduced on or adjacent to existing heart tissue in a subject. In one embodiment, a CVP cell composition or tissue engineered myocardium composition as disclosed herein is useful for replacing damaged heart tissue (e.g., ischemic heart tissue), for example, where a CVP cell composition or tissue engineered myocardium composition as disclosed herein is introduced or administered (e.g. implanted) into a subject. In some embodiments, the tissue engineered myocardium composition which is transplanted comprises CVP cells originated and derived from the subject in which the tissue engineered myocardium is implanted. Accordingly, allogenic or autologous transplantation of the tissue engineered myocardium into a subject can be carried out.

Another aspect of the present invention provides methods of treating a cardiac disorder in a subject, the method generally involving administering to a subject in need thereof a therapeutically effective amount of a CVP cell composition or tissue engineered myocardium composition as disclosed herein. In some embodiments, the present invention also provides methods of treating a cardiac disorder in a subject, the method generally involving administering to a subject in need thereof a therapeutically effective amount of a substantially pure population of CVP cells as disclosed herein.

In some embodiments, the CVP cell composition or tissue engineered myocardium composition as disclosed herein is useful for generating artificial heart tissue, e.g., for implanting into a mammalian subject. In some embodiments, the CVP cell composition or tissue engineered myocardium composition as disclosed herein is useful for replacing damaged heart tissue (e.g., ischemic heart tissue). Accordingly, one can use of the tissue engineered myocardium composition as described herein to repair and/or reinforce the cardiac or heart tissue in a mammal, e.g., an injured or diseased human subject. For example, in some embodiments a CVP cell-seeded film/polymer can be used, for example but not limited to, in tissue implants or as a patch or as reinforcement to a heart which is weak contraction or alternatively has been damaged due to a myocardial infarction, and/or as a wound dressing. Such wound dressing can offer improved cardiac function of a subject with a cardiac lesion such as myocardial infarction. The tissue engineered myocardial composition as disclosed herein is also useful to repair other tissue defects, e.g., for cardiac repair due to birth defects (congenic) or acquired cardiac defects, or to function as a splint for damaged or weakened muscle, for example in degenerative muscular disorders where muscle atrophy of the heart occurs, such as multiple sclerosis (MS), ALS and muscular dystrophy and the like. In some embodiments, the tissue engineered myocardium compositions are portable and amenable to both hospital (e.g., operating room) use as well as field (e.g., battlefield) use. The tissue engineered myocardium compositions are easily transported, for instance, films or polymers are packaged wet or dry, e.g., cell scaffold/net alone, net+CVP cells, or net+CVP cells+drug (e.g., antibiotic, blood coagulant or anti-coagulant). A net is characterized by a pattern or mesh of filaments or threads. The filaments or threads are organized into a grid structure or are present in an amorphous tangle. The film is peeled away from a support and applied to injured or diseased tissue.

In one embodiment, a method of using a tissue engineered myocardium composition as disclosed herein optionally includes a step of wrapping the biopolymer structure around a three-dimensional implant and then inserting the implant into a subject. For example, the tissue engineered myocardium composition is placed on or in the heart, e.g. on or near a cardiac muscle tissue in need of improved and/or strengthening. The substrate, e.g., metal, ceramic, polymer or a combination thereof, is characterized as having an elastic modulus is greater than 1 MPa. For example, the substrate is selected from a glass cover slip, polystyrene, polymethylmethacrylate, polyethylene terephthalate film, gold and a silicon wafer. In some embodiments, the scaffold can be removed prior to implantinf a MTF into a subject. In some embodiments, CVP cells can be seeded onto a scaffold of any geometric shape, such as a spiral or V-shaped, or O-shapped scaffold, such that the CVP cells form myocardial tissue which conforms to the same shape of the scaffold. Once the scaffold is no longer present (e.g. by physical removal or bioabsorbtion of a biodegradable and/or bioabsorbable substrate) the CVPs remain in the shape of the scaffold. In one embodiment, where the scaffold is a spiral geometry, the myocardial tissue generated by the CVPs will effectively form a “contracting spiral” conformation. In another embodiment, the scaffold may be in a geometric shape such that CVPs in effect form an engineered biological pincer, where the CVPs are seeded onto a scaffold of 2 arms of a “V” shape, which are joined in the centre, allowing the free arms of the V to come into contact when the MTF contracts. In another embodiment, a hollow tube of engineered myocardial tissue can be formed by seeding on a scaffold shaped as a cylinder. In some embodiments, the engineered myocardium generated using the methods as disclosed herein can be implanted into a subject. In alternative embodiments, the engineered myocardium can be used for any useful means, such as a fishing lure and the like to aid catching fish.

A subject in need of treatment using a subject method include, but are not limited to, individuals having a congenital heart defect; individuals suffering from a condition that results in ischemic heart tissue, e.g., individuals with coronary artery disease; and the like. A subject method is useful to treat degenerative muscle disease, e.g., familial cardiomyopathy, dilated cardiomyopathy, hypertrophic cardiomyopathy, restrictive cardiomyopathy, or coronary artery disease with resultant ischemic cardiomyopathy.

For administration to a mammalian host, the CVP cell composition or tissue engineered myocardium composition as disclosed herein can be formulated as a pharmaceutical composition. A pharmaceutical composition can be a sterile aqueous or non-aqueous solution, suspension or emulsion, which additionally comprises a physiologically acceptable carrier (e.g., a non-toxic material that does not interfere with the activity of the active ingredient). Any suitable carrier known to those of ordinary skill in the art may be employed in a subject pharmaceutical composition. The selection of a carrier will depend, in part, on the nature of the substance (e.g., cells or chemical compounds) being administered. Representative carriers include physiological saline solutions, gelatin, water, alcohols, natural or synthetic oils, saccharide solutions, glycols, injectable organic esters such as ethyl oleate or a combination of such materials. Optionally, a pharmaceutical composition may additionally contain preservatives and/or other additives such as, for example, antimicrobial agents, anti-oxidants, chelating agents and/or inert gases, and/or other active ingredients.

In some embodiments, where CVP cells are administered to a subject in need thereof, a population of CVP cells are encapsulated, according to known encapsulation technologies, including microencapsulation (see, e.g., U.S. Pat. Nos. 4,352,883; 4,353,888; and 5,084,350, which are incorporated herein by reference). Where the CVP cells are encapsulated, in some embodiments the CVP cells are encapsulated by macroencapsulation, as described in U.S. Pat. Nos. 5,284,761; 5,158,881; 4,976,859; 4,968,733; 5,800,828 and published PCT patent application WO 95/05452 which are incorporated herein by reference. A unit dosage form of a CVP population can contain from about 10³ cells to about 10⁹ cells, e.g., from about 10³ cells to about 10⁴ cells, from about 10⁴ cells to about 10⁵ cells, from about 10⁵ cells to about 10⁶ cells, from about 10⁶ cells to about 10⁷ cells, from about 10⁷ cells to about 10⁸ cells, or from about 10⁸ cells to about 10⁹ cells.

A tissue engineered myocardium composition as disclosed herein, or a CVP population as disclosed herein can be cryopreserved according to routine procedures. For example, cryopreservation can be carried out on from about one to ten million cells in “freeze” medium which can include a suitable proliferation medium, 10% BSA and 7.5% dimethylsulfoxide. Cells are centrifuged. Growth medium is aspirated and replaced with freeze medium. Cells are resuspended as spheres. Cells are slowly frozen, by, e.g., placing in a container at −80° C. Cells are thawed by swirling in a 37° C. bath, resuspended in fresh proliferation medium, and grown as described above.

As discussed above, the tissue engineered myocardium composition or CVP compostion as disclosed herein can be used as a pharmaceutical composition to the treatment of a subject in need thereof, for example for the treatment of a subject with a cardiomyopathy or a cardiovascular condition or disease. In some embodiments, a CVP cell composition or tissue engineered myocardium composition as disclosed herein may further comprise a CVP differentiation agent, which promotes the differentiation of CVP into ventricular cardiomyoctyes. Cardiovascular stem cell differentiation agents for use in the present invention are well known to those of ordinary skill in the art. Examples of such agents include, but are not limited to, cardiotrophic agents, creatine, carnitine, taurine, cardiotropic factors as disclosed in U.S. Patent Application Serial No. 2003/0022367 which is incorporated herein by reference, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the BMP or cripto pathway. The pharmaceutical compositions may further comprise a pharmaceutically acceptable carrier.

A CVP cell composition or tissue engineered myocardium composition as disclosed herein can be applied alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population. The CVP cell population of the CVP cell composition or tissue engineered myocardium composition as disclosed herein may also be modified by insertion of DNA to modify the function of the cells for structural and/or therapeutic purpose. As discussed herein, gene transfer techniques for stem cells are known by persons of ordinary skill in the art, as disclosed in (Morizono et al., 2003; Mosca et al., 2000), and can include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In another aspect, CVP cells present in a CVP cell composition or tissue engineered myocardium composition as disclosed herein for transplantation can be modified to comprise a gene encoding pro-angiogenic and/or cardiomyogenic growth factor(s) which would allow the CVP cells to act as their own source of growth factor during cardiac repair or regeneration following transplantation into a subject. Genes encoding anti-apoptotic factors or agents could also be applied. Addition of the gene (or combination of genes) could be by any technology known in the art including but not limited to adenoviral transduction, “gene guns,” liposome-mediated transduction, and retrovirus or lentivirus-mediated transduction, plasmid' adeno-associated virus. CVP cells could be genetically manipulated to release and/or express genes for a defined period of time (such that gene expression could be induced and/or controlled, so expression can be contined and/or be initiated. Particularly, when a CVP cell composition or tissue engineered myocardium composition as disclosed herein is administered to a subject other than the subject from whom the cells and/or tissue were obtained, one or more immunosuppressive agents may be administered to the subject receiving a CVP cell composition or tissue engineered myocardium composition as disclosed herein in order to reduce, and preferably prevent, rejection of the transplant by the recipient subject. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 20020182211. In one embodiment, a immunosuppressive agent is cyclosporine A. Other examples include myophenylate mofetil, rapamicin, and anti-thymocyte globulin. In one embodiment, an immunosuppressive drug is administered with at least one other therapeutic agent. An immunosuppressive agent can be administered to a subject in a formulation which is compatible with the route of administration and is administered to a subject at a dosage sufficient to achieve the desired therapeutic effect. In another embodiment, an immunosuppressive agent is administered transiently for a sufficient time to induce tolerance of the CVP cell composition or tissue engineered myocardium composition as disclosed herein.

In some embodiments, a CVP cell composition or tissue engineered myocardium composition as disclosed herein can be administered to a subject with one or more cellular differentiation agents, such as cytokines and growth factors, as disclosed herein. Examples of various cell differentiation agents are disclosed in U.S. Patent Application Serial No. 2003/0022367 which is incorporated herein by reference, or Gimble et al., 1995; Lennon et al., 1995; Majumdar et al., 1998; Caplan and Goldberg, 1999; Ohgushi and Caplan, 1999; Pittenger et al., 1999; Caplan and Bruder, 2001; Fukuda, 2001; Worster et al., 2001; Zuk et al., 2001. Other examples of cytokines and growth factors include, but are not limited to, cardiotrophic agents, creatine, carnitine, taurine, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the BMP or cripto pathway.

A CVP cell composition or tissue engineered myocardium composition as disclosed herein can be administered to a subject in need of a transplant. In other aspects of the present invention, a CVP cell composition or tissue engineered myocardium composition as disclosed herein is directly administered at the site of or in proximity to the diseased and/or damaged tissue. A CVP cell composition or tissue engineered myocardium composition as disclosed herein for therapeutic transplantation purposes can optionally be packaged in a suitable container with written instructions for a desired purpose, such as the use of the CVP cell composition or tissue engineered myocardium composition as disclosed herein to improve some abnormality of the cardiac muscle, in particular the right ventricle of the heart.

In one embodiment, a subject can be administered a CVP cell composition or tissue engineered myocardium composition as disclosed herein and also administered, either in conjunction or temporally separated a differentiation agent. In one embodiment, a CVP cell composition or tissue engineered myocardium composition as disclosed herein is administered separately to the subject from the differentiation agent. Optionally, if a CVP cell composition or tissue engineered myocardium composition as disclosed herein is administered separately from the differentiation agent, there is a temporal separation in the administration of the a tissue engineered myocardium composition and the differentiation agent. The temporal separation may range from about less than a minute in time, to about hours or days in time. The determination of the optimal timing and order of administration is readily and routinely determined by one of ordinary skill in the art.

The CVP Cell Composition or Tissue Engineered Myocardium Composition as Disclosed Herein to Generate Artificial Heart Tissue

In some embodiments, the present invention provides a tissue engineered myocardium composition and a method for generating such tissue engineered myocardium composition in vitro for use and implanting the artificial heart tissue in vivo.

The CVP cell composition or tissue engineered myocardium composition as disclosed herein can be used for allogenic or autologous transplantation into an subject in need thereof. To produce a CVP cell composition or tissue engineered myocardium composition as disclosed herein, a substrate can be provided which is brought into contact with the CVP cells, where the CVP cells give rise to ventricular cardiomyocytes.

Pharmaceutical Compositions

The present invention provides tissue engineered myocardium compositions generated using a CVP cells and a suitable substrate such as subject method. In some embodiments, the tissue engineered myocardium composition is muscle thin film (MTF) tissue. In alternative embodiments, the tissue engineered myocardium composition is artificial heart tissue.

In some embodiments, a tissue engineered myocardium is present in a liquid medium together with one or more components. Suitable components include, but are not limited to, salts; buffers; stabilizers; protease-inhibiting agents; cell membrane- and/or cell wall-preserving compounds, e.g., glycerol, dimethylsulfoxide, etc.; nutritional media appropriate to the cell; and the like.

The tissue engineered myocardium as disclosed herein can be used for allogenic or autologous transplantation into an individual in need thereof. To produce tissue engineered myocardium, a scaffold or support can be provided which is brought into contact with the CVP cells as disclosed herein.

The term “support” should be understood in this connection to mean any suitable carrier material to which the cells are able to attach themselves or adhere in order to form the corresponding cell composite, e.g. the artificial tissue. In some embodiments, the matrix or carrier material, respectively, is present already in a three-dimensional form desired for later application. For example, bovine pericardial tissue is used as matrix which is crosslinked with collagen, decellularized and photofixed.

For example, a scaffold (also referred to as a “biocompatible substrate”) is a material that is suitable for implantation into a subject onto which a cell population can be deposited. A biocompatible substrate does not cause toxic or injurious effects once implanted in the subject. In one embodiment, the biocompatible substrate is a polymer with a surface that can be shaped into the desired structure that requires repairing or replacing. The polymer can also be shaped into a part of a structure that requires repairing or replacing. The biocompatible substrate provides the supportive framework that allows cells to attach to it, and grow on it. Cultured populations of cells can then be grown on the biocompatible substrate, which provides the appropriate interstitial distances required for cell-cell interaction.

Uses of CVP Cells

In one embodiment of the invention, a CVP cell as disclosed herein can be used as an assay for the study and understanding of signaling pathways secondary heart field progenitors, such as their growth and differentiation, particularly with respect to cardiomyocytes such as ventricular cardiomyocytes. The use of a CVP cells of the present invention is useful to aid the development of therapeutic applications for cardiomyopathy and other cardiovascular diseases as well as congenital and adult heart failure. The use of such CVP cells of the invention enable the study of secondary heart field lineages, in particular the development and differentiation of cells to generate cardiac structures such as the right ventricle (RV) and the outflow tract (OFT) without the need and complexity of time consuming animal models. In another embodiment, the CVP cells as disclosed herein can be genetically modified to carry specific disease and/or pathological traits and phenotypes of cardiomyogenic diseases, cardiomyopathies, cardiac disease and adult heart failure.

In one embodiment, CVP cells can be used in assays to study their function and development, and in some embodiments, such CVP cells are derived from ES sources or iPS cell sources. In one embodiment, the CVP cells as disclosed herein can be used for the study of differentiation pathways of cardiomyocytes, such as ventricular cardiomyocytes. In one embodiment, subpopulations of CVP cells can be studied, for example, study of subpopulations of CVP cells which differentiate into ventricular cardiomyocytes which form the right ventricle (RV) and those ventricular cardiomyocytes which form into outflow tract (OFT) cardiomyocytes, conduction system cardiomyocytes.

In another embodiment, CVP cells can also be used for the study of CVP cell which comprise a pathological characteristic, for example, a disease and/or genetic characteristic associated with a disease or disorder. In some embodiments, the disease of disorder is a cardiovascular disorder or disease. In some embodiments, the cardiovascular stem cell has been genetically engineered to comprise the characteristic associated with a disease or disorder. Such methods to genetically engineer the cardiovascular stem cell are well known by those in the art, and include introducing nucleic acids into the cell by means of transfection, for example but not limited to use of viral vectors or by other means known in the art.

As discussed above, CVP cells as disclosed herein can be easily manipulated by one of ordinary skill in the art in experimental systems that offer the advantages of targeted lineage differentiation as well as clonal homogeneity and the ability to manipulate external environments. Furthermore, due to ethical unacceptability of experimentally altering a human germ line, the human ES-derived CVP cells or iPS-derived CVP cells for use in the tissue engineered myocardium as disclosed herein is especially beneficial. Gene targeting in human CVP cells, such as ES-derived CVP cells or iPS-derived CVP cells allows important applications in areas where rodent model systems do not adequately recapitulate human biology or disease processes.

In another embodiment, the CVP cells as isolated and identified herein can be used to prepare a cDNA library relatively uncontaminated with cDNA that is preferentially expressed in cells from other lineages. For example, CVP cells are collected and then mRNA is prepared from the pellet by standard techniques (Sambrook et al., supra). After reverse transcribing into cDNA, the preparation can be subtracted with cDNA from other undifferentiated ES cells, other progenitor cells, or end-stage cells from the cardiomyocyte or any other developmental pathway, for example, in a subtraction cDNA library procedure. Furthermore, CVP cells of this invention can also be used to prepare antibodies that are specific for markers of the CVP cells and their precursors. Polyclonal antibodies can be prepared by injecting a vertebrate animal with cells of this invention in an immunogenic form. Production of monoclonal antibodies is described in such standard references as U.S. Pat. Nos. 4,491,632, 4,472,500 and 4,444,887, and Methods in Enzymology 73B:3 (1981). Specific antibody molecules can also be produced by contacting a library of immunocompetent cells or viral particles with the target antigen, and growing out positively selected clones. See Marks et al., New Eng. J. Med. 335:730, 1996, and McGuiness et al., Nature Biotechnol. 14:1449, 1996. A further alternative is reassembly of random DNA fragments into antibody encoding regions, as described in EP patent application 1,094,108 A.

The antibodies in turn can be used to identify or rescue (for example restore the phenotype) cells of a desired phenotype from a mixed cell population, for purposes such as co-staining during immunodiagnosis using tissue samples, and isolating precursor cells from terminally differentiated cardiomyocytes and cells of other lineages. Of particular interest is the examination of the gene expression profile during and following differentiation of the cardiovascular stem cells of the invention. The expressed set of genes may be compared against other subsets of cells, against ES cells, against adult heart tissue, and the like, as known in the art. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+mRNA. One of skill in the art can readily use these methods to determine differences in the molecular size or amount of mRNA transcripts between two samples.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein. Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population. Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680. Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505. Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined. Methods for analyzing the data collected from hybridization to arrays are well known in the art. For example, where detection of hybridization involves a fluorescent label, data analysis can include the steps of determining fluorescent intensity as a function of substrate position from the data collected, removing outliers, e.g. data deviating from a predetermined statistical distribution, and calculating the relative binding affinity of the targets from the remaining data. The resulting data can be displayed as an image with the intensity in each region varying according to the binding affinity between targets and probes. Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992. General methods in molecular and cellular biochemistry can also be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following written description provides exemplary methodology and guidance for carrying out many of the varying aspects of the present invention.

Molecular Biology Techniques: Standard molecular biology techniques known in the art and not specifically described are generally followed as in Sambrook et al., Molecular Cloning: A Laboratory Manual, Cold Springs Harbor Laboratory, N.Y. (1989, 1992), and in Ausubel et al., Current Protocols in Molecular Biology, John Wiley and Sons, Baltimore, Md. (1989). Polymerase chain reaction (PCR) is carried out generally as in PCR Protocols: A Guide to Methods and Applications, Academic Press, San Diego, Calif. (1990). Reactions and manipulations involving other nucleic acid techniques, unless stated otherwise, are performed as generally described in Sambrook et al., Molecular Cloning: A Laboratory Manual, Cold Springs Harbor Laboratory Press, and methodology as set forth in U.S. Pat. Nos. 4,666,828; 4,683,202; 4,801,531; 5,192,659; and 5,272,057 and incorporated herein by reference. In situ PCR in combination with Flow Cytometry can be used for detection of cells containing specific DNA and mRNA sequences (see, for example, Testoni et al., Blood, 1996, 87:3822).

Immunoassays: Standard methods in immunology known in the art and not specifically described are generally followed as in Stites et al. (Eds.), Basic And Clinical Immunology, 8th Ed., Appleton & Lange, Norwalk, Conn. (1994); and Mishell and Shigi (Eds.), Selected Methods in Cellular Immunology, W. H. Freeman and Co., New York (1980).

In general, immunoassays are employed to assess a specimen such as for cell surface markers or the like. Immunocytochemical assays are well known to those skilled in the art. Both polyclonal and monoclonal antibodies can be used in the assays. Where appropriate other immunoassays, such as enzyme-linked immunosorbent assays (ELISAs) and radioimmunoassays (RIA), can be used as are known to those in the art. Available immunoassays are extensively described in the patent and scientific literature. See, for example, U.S. Pat. No. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771; and 5,281,521 as well as Sambrook et al., Molecular Cloning: A Laboratory Manual, Cold Springs Harbor, N.Y., 1989. Numerous other references also may be relied on for these teachings.

Further elaboration of various methods that can be utilized for quantifying the presence of the desired marker include measuring the amount of a molecule that is present. A convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein (GFP) chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

Antibody Production: Antibodies may be monoclonal, polyclonal, or recombinant. Conveniently, the antibodies may be prepared against the immunogen or immunogenic portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane, Antibodies: A Laboratory Manual, Cold Spring Harbor Laboratory, Cold Springs Harbor, N.Y. (1988) and Borrebaeck, Antibody Engineering—A Practical Guide by W. H. Freeman and Co. (1992). Antibody fragments may also be prepared from the antibodies and include Fab and F(ab′)2 by methods known to those skilled in the art. For producing polyclonal antibodies a host, such as a rabbit or goat, is immunized with the immunogen or immunogenic fragment, generally with an adjuvant and, if necessary, coupled to a carrier; antibodies to the immunogen are collected from the serum. Further, the polyclonal antibody can be absorbed such that it is monospecific. That is, the serum can be exposed to related immunogens so that cross-reactive antibodies are removed from the serum rendering it monospecific.

For producing monoclonal antibodies, an appropriate donor is hyperimmunized with the immunogen, generally a mouse, and splenic antibody-producing cells are isolated. These cells are fused to immortal cells, such as myeloma cells, to provide a fused cell hybrid that is immortal and secretes the required antibody. The cells are then cultured, and the monoclonal antibodies harvested from the culture media.

For producing recombinant antibodies, messenger RNA from antibody-producing B-lymphocytes of animals or hybridoma is reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker The antibody, or antibody fragment, is expressed using a suitable expression system. Antibody cDNA can also be obtained by screening pertinent expression libraries. The antibody can be bound to a solid support substrate or conjugated with a detectable moiety or be both bound and conjugated as is well known in the art. (For a general discussion of conjugation of fluorescent or enzymatic moieties see Johnstone & Thorpe, Immunochemistry in Practice, Blackwell Scientific Publications, Oxford, 1982). The binding of antibodies to a solid support substrate is also well known in the art. (see for a general discussion Harlow & Lane, Antibodies: A Laboratory Manual, Cold Spring Harbor Laboratory Publications, New York, 1988 and Borrebaeck, Antibody Engineering—A Practical Guide, W. H. Freeman and Co., 1992). The detectable moieties contemplated with the present invention can include, but are not limited to, fluorescent, metallic, enzymatic and radioactive markers. Examples include biotin, gold, ferritin, alkaline phosphates, galactosidase, peroxidase, urease, fluorescein, rhodamine, tritium, 14C, iodination and green fluorescent protein.

Gene therapy and genetic engineering of cardiovascular stem cells and/or mesenchymal cells: Gene therapy as used herein refers to the transfer of genetic material (e.g., DNA or RNA) of interest into a host to treat or prevent a genetic or acquired disease or condition. The genetic material of interest encodes a product (e.g., a protein, polypeptide, and peptide, functional RNA, antisense, RNA, microRNA, siRNA, shRNA, PNA, pcPNA) whose in vivo production is desired. For example, the genetic material of interest encodes a hormone, receptor, enzyme polypeptide or peptide of therapeutic value. Alternatively, the genetic material of interest encodes a suicide gene. For a review see “Gene Therapy” in Advances in Pharmacology, Academic Press, San Diego, Calif., 1997.

With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998). With respect to the culture of heart cells, standard references include The Heart Cell in Culture (A. Pinson ed., CRC Press 1987), Isolated Adult Cardiomyocytes (Vols. I & II, Piper & Isenberg eds, CRC Press 1989), Heart Development (Harvey & Rosenthal, Academic Press 1998).

The present invention is further illustrated by the following examples which in no way should be construed as being further limiting, The contents of all cited references, including literature references, issued patents, published patent applications, and co-pending patent applications, cited throughout this application are hereby expressly incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

In some embodiments of the present invention may be defined in any of the following numbered paragraphs:
1. A composition comprising a substantially pure population of committed ventricular progenitors (CVP), wherein a CVP is positive for the expression of Mef2c+ and Nkx2.5+ and is capable of differentiating into the right ventricle (RV) and/or outflow tract (OT),
2. The composition of paragraph 1, wherein the CVP is positive for the expression of marker genes selected from the group consisting of: Isl1+, Tbx20, GATA4, GATA6, TropininT, Troponin C, BMP7, BMP4 and BMP2.
3. The composition of paragraphs 1 or 2, wherein the CVP is positive for the expression of an miRNA selected from the group consisting of: miRNA-208, miR-143, miR-133a, miR-133b, miR-1, miR-143 and miR-689.
4. The composition of paragraph 1, wherein the CVP is derived from an ES cell.
5. The composition of any of paragraphs 1 to 4, wherein the CVP is genetically modified.
6. The composition of any of paragraphs 1 to 5, wherein the CVP is a mammalian cell.
7. The composition of paragraph 6, wherein the mammalian cell is a human cell.
8. The composition of paragraph 1, wherein the CVP is capable of differentiating into a ventricular cardiomyocyte.
9. The composition of paragraph 1, wherein the composition comprises at least one CVP cell which has a pathological characteristic of a disease or disorder.
10. The composition of paragraph 9, wherein the pathological characteristic is a mutation or polymorphism.
11. The composition of paragraph 9, wherein the pathological characteristic is a genetically engineered pathological characteristic.
12. The composition of paragraph 9, wherein the disease is a cardiac dysfunction.
13. The composition of paragraph 12, wherein the cardiac dysfunction is congestive heart failure.
14. The composition of paragraph 13, wherein the congestive heart failure is congenic congestive heart failure.
15. The composition of paragraph 9, wherein the disease is myocardial infarction.
16. The composition of paragraph 9, wherein the disease is endogenous myocardial regeneration.
17. The composition of paragraph 9, wherein the disease is selected from the group consisting of: atherosclerosis; cardiomyopathy; congenital heart disease; hypertension; blood flow disorders; symptomatic arrhythmia; pulmonary hypertension; dysfunction in conduction system; dysfunction in coronary arteries; dysfunction in coronary arterial tree and coronary artery catheterization.
18. A method of treating a cardiovascular disorder in a subject in need thereof, comprising administering an effective amount of the composition of paragraph 1.
19. A method to enhance cardiac function in a subject in need thereof, comprising administering an effective amount of the composition of paragraph 1 to enhance cardiac function.
20. Use of the composition of paragraph 1 for the treatment of a cardiovascular disease or disorder in a subject, wherein the composition is administered by transplantation to the subject in need of treatment.
21. The method of any of paragraphs 18 to 20, wherein the myocardial tissue comprises CVPs obtained from a mammalian subject.
22. The method of paragraph 21, wherein the mammalian subject is a human subject.
23. The method of paragraph 21, wherein the CVPs are obtained from the same subject as the subject to which the composition is administered.
24. The method of any of paragraphs 18 to 20, wherein the subject suffers from, or is at risk of developing, a disease or disorder characterized by insufficient cardiac function.
25. The method of paragraph 24, wherein the disease or disorder is selected from the group consisting of: congestive heart failure, coronary artery disease, myocardial infarction, myocardial ischemia, tissue ischemia, cardiac ischemia, vascular disease, acquired heart disease, congenital heart disease, atherosclerosis, cardiomyopathy, dysfunctional conduction systems, dysfunction in coronary arteries, cardiomyopathy, idiopathic cardiomyopathy, cardiac arrhythmias, symptomatic arrhythmia, muscular dystrophy, muscle mass abnormality, muscle degeneration, infective myocarditis, drug- or toxin-induced muscle abnormalities, hypersensitivity myocarditis, an autoimmune endocarditis, dysfunctional coronary arteries, pulmonary heart hypertension, atrial and ventricular arrhythmias, hypertensive vascular diseases, peripheral vascular diseases, hypertension; blood flow disorders; pulmonary hypertension; dysfunction in coronary arterial tree and coronary artery colaterization.
26. The method of any of paragraphs 18 to 25, wherein the subject is a mammal.
27. The method of paragraph 26, wherein the mammal is a human.
28. Use of the composition of paragraph 1 in an assay to identify a cardiotoxic agent.
29. The use of paragraph 28, wherein an agent which decreases the contractile activity of the composition of paragraph 1 is a cardiotoxic agent.
30. The use of paragraph 28, wherein an agent which increases the contractile activity of the composition of paragraph 1 is a cardiotoxic agent.
31. The use of paragraphs 29 or 30, wherein the contractile activity is selected from the group consisting of: contractile force, contractile frequency, contractile duration and contractile stamina.
32. A composition comprising a substantially pure population of committed ventricular progenitors (CVP) and a scaffold, wherein the CVP cell is positive for the expression of Mef2c+ and Nkx2.5+ and is capable of differentiating into the right ventricle (RV) and/or outflow tract (OT),
33. The composition of paragraph 32, wherein the scaffold comprises a plurality of freestanding tissue structures, wherein each free standing tissue structure comprises a flexible polymer scaffold imprinted with a predetermined pattern, and the CVPs are arranged in spatially organized manner according to said pattern to yield contractible myocardial tissue.
34. The composition of paragraph 32, where in the scaffold is a biocompatible substrate
35. The composition of paragraphs 32 or 34, wherein the biocompatible substrate is biodegradable.
36. The composition of paragraph 32, where in the scaffold is a two-dimensional scaffold.
37. The composition of paragraph 32, where in the scaffold is a three-dimensional scaffold.
38. The composition of paragraphs 32 or 37, wherein the three-dimensional scaffold is a plurality of two dimensional scaffold.
39. The composition of paragraph 33, wherein the patterned biopolymer structure is a freestanding biopolymer comprising an integral pattern of the biopolymer having repeating features with a dimension of less than 1 mm and without a supporting substrate.
40. The composition of paragraph 33 or 39, wherein the free-standing biopolymer structure has repeating features with a dimension of 100 nm or less.
41. The composition of any of paragraphs 32 to 40, wherein the scaffold comprises at least one biopolymer selected from extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, and combinations thereof.
42. The composition of paragraphs 33 or 39, wherein the free-standing biopolymer structure comprises an integral pattern of the biopolymer and poly(N-Isopropylacrylamide).
43. The composition of paragraph 32, wherein the scaffold is selected from the group consisting of: collagen, poly (alpha esters), poly(lactate acid), poly(glycolic acid), polyorthesters, polyanhydrides, cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, or copolymers or physical blends thereof.
44. The composition of paragraph 32, wherein the CVP is positive for the expression of marker genes selected from the group consisting of: Isl1+, Tbx20, GATA4, GATA6, TropininT, Troponin C, BMP7, BMP4 and BMP2.
45. The composition of any of paragraphs 32 to 44, wherein the CVP is positive for the expression of an miRNA selected from the group consisting of: miRNA-208, miR-143, miR-133a, miR-133b, miR-1, miR-143 and miR-689.
46. The composition of any of paragraphs 32 to 45, wherein the CVP is derived from an ES cell.
47. The composition of any of paragraphs 32 to 46, wherein the CVP is genetically modified.
48. The composition of any of paragraphs 32 to 47, wherein the CVP is a mammalian cell.
49. The composition of paragraph 48, wherein the mammalian cell is a human cell.
50. The composition of paragraph 32, wherein the CVP is capable of differentiating into a ventricular cardiomyocyte.
51. The composition of paragraph 32, wherein the composition comprises at least one CVP cell which has a pathological characteristic of a disease or disorder.
52. The composition of paragraph 51, wherein the pathological characteristic is a mutation or polymorphism.
53. The composition of paragraph 51, wherein the pathological characteristic is a genetically engineered pathological characteristic.
54. The composition of paragraph 51, wherein the disease is a cardiac dysfunction.
55. The composition of paragraph 54, wherein the cardiac dysfunction is congestive heart failure.
56. The composition of paragraph 55, wherein the congestive heart failure is congenic congestive heart failure.
57. The composition of paragraph 51, wherein the disease is myocardial infarction.
58. The composition of paragraph 51, wherein the disease is endogenous myocardial regeneration.
59. The composition of paragraph 51, wherein the disease is selected from the group consisting of: atherosclerosis; cardiomyopathy; congenital heart disease; hypertension; blood flow disorders; symptomatic arrhythmia; pulmonary hypertension; dysfunction in conduction system; dysfunction in coronary arteries; dysfunction in coronary arterial tree and coronary artery catheterization.
60. A method to identify an agent that alters the contractile activity of myocardial tissue, comprising:
a. contacting the myocardial tissue of paragraph 32 with at least one agent;
b. measuring the contractile activity of the myocardial tissue in the presence of at least one agent;
c. comparing the contractile activity of the myocardial tissue in the presence of at least one agent with a reference contractile activity of myocardial tissue;
wherein a change in the contractile activity in the presence of the agent as compared to the reference contractile activity identifies an agent that alters the contractile activity.
61. The method of paragraph 60, wherein a change in the contractile activity is an increase in contractile activity.
62. The method of paragraph 60, wherein a change in the contractile activity is a decrease in contractile activity.
63. The method of paragraph 60, wherein the contractile activity is selected from the group consisting of: contractile force, contractile frequency, contractile duration and contractile stamina.
64. The method of paragraph 60, wherein the reference contractile activity is the contractile activity of the myocardial tissue of paragraph 1 in the absence of an agent.
65. The method of paragraph 60, wherein the reference contractile activity is the contractile activity of the myocardial tissue of paragraph 1 in the presence of at least one positive control agent.
66. The method of paragraph 60, wherein the reference contractile activity is the contractile activity of the myocardial tissue of paragraph 1 in the presence of at least one negative control agent.
67. A method for generating contractile myocardial tissue, comprising contacting a plurality of committed ventricular progenitors (CVP) with a surface of a scaffold, wherein the CVP is positive for the expression of Mef2c+ and Nkx2.5+, and whereby the alignment of the CVPs in a spatially organized manner on the surface of the scaffold forms contractile myocardial tissue.
68. The method of paragraph 67, wherein the CVP is positive for the expression of marker genes selected from the group consisting of: Isl1+, Tbx20, GATA4, GATA6, TropininT, Troponin C, BMP7, BMP4 and BMP2.
69. The method of any of paragraphs 67 to 68, wherein the CVP is positive for the expression of miRNAs selected from the group consisting of: miRNA-208, miR-143, miR-133a, miR-133b, miR-1, miR-143 and miR-689.
70. The method of paragraph 67, wherein the CVP is derived from an ES cell.
71. The method of any of paragraphs 67 to 70, wherein the CVP is a genetically modified cell.
72. The method of any of paragraphs 67 to 71, wherein the CVP is a mammalian cell.
73. The method of any of paragraphs 67 to 72, wherein the mammalian cell is a human cell.
74. The method of any of paragraphs 67 to 73, wherein the CVP is capable of differentiating into a ventricular cardiomyocyte.
75. The method of any of paragraphs 67 to 74, wherein a population of CVPs or CVP-derived ventricular cardiomyocytes are arranged in a spatially organized manner on the free-standing structures so that the CVPs or CVP-derived ventricular cardiomyocytes are aligned in an uni-axial cell arrangement.
76. The method of paragraph 67, wherein the scaffold is a plurality of freestanding tissue structures.
77. The method of paragraph 76, wherein each free-standing tissue structure comprises a flexible polymer scaffold imprinted with a predetermined pattern, and the CVPs are arranged in spatially organized manner according to said pattern to yield contractible myocardial tissue.
78. The method of paragraph 67, wherein the scaffold is a biopolymer structure.
79. The method of paragraph 78, wherein the biopolymer structure has repeating features with a dimension of 100 nm or less.
80. The method of any of the paragraphs 67 to 79, wherein the scaffold comprises at least one biopolymer selected from extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, and combinations thereof.
81. The method of paragraphs 67, wherein the biopolymer structure comprises an integral pattern of the biopolymer and poly(N-Isopropylacrylamide).
82. A method of treating a cardiovascular disorder in a subject in need thereof, comprising administering an effective amount of the composition of paragraph 32.
83. A method to enhance cardiac function in a subject in need thereof, comprising administering an effective amount of the composition of paragraph 32 to enhance cardiac function.
84. Use of the composition of paragraph 32 for the treatment of a cardiovascular disease or disorder in a subject, wherein the composition is administered by transplantation to the subject in need of treatment.
85. The method of any of paragraphs 82 to 83, wherein the myocardial tissue comprises CVPs obtained from a mammalian subject.
86. The method of paragraph 85, wherein the mammalian subject is a human subject.
87. The method of paragraph 85, wherein the CVPs are obtained from the same subject as the subject to which the composition is administered.
88. The method of any of paragraphs 82 or 83, wherein the subject suffers from, or is at risk of developing, a disease or disorder characterized by insufficient cardiac function.
89. The method of paragraph 88, wherein the disease or disorder is selected from the group consisting of: congestive heart failure, coronary artery disease, myocardial infarction, myocardial ischemia, tissue ischemia, cardiac ischemia, vascular disease, acquired heart disease, congenital heart disease, atherosclerosis, cardiomyopathy, dysfunctional conduction systems, dysfunction in coronary arteries, cardiomyopathy, idiopathic cardiomyopathy, cardiac arrhythmias, symptomatic arrhythmia, muscular dystrophy, muscle mass abnormality, muscle degeneration, infective myocarditis, drug- or toxin-induced muscle abnormalities, hypersensitivity myocarditis, an autoimmune endocarditis, dysfunctional coronary arteries, pulmonary heart hypertension, atrial and ventricular arrhythmias, hypertensive vascular diseases, peripheral vascular diseases, hypertension; blood flow disorders; pulmonary hypertension; dysfunction in coronary arterial tree and coronary artery colaterization.
90. The method of any of paragraphs 82 to 89, wherein the subject is a mammal.
91. The method of paragraph 90, wherein the mammal is a human.
92. Use of the composition of paragraph 32 in an assay to identify a cardiotoxic agent.
93. The use of paragraph 92, wherein an agent which decreases the contractile activity of the composition of paragraph 1 is a cardiotoxic agent.
94. The use of paragraph 92, wherein an agent which increases the contractile activity of the composition of paragraph 1 is a cardiotoxic agent.
95. The use of paragraphs 93 or 99, wherein the contractile activity is selected from the group consisting of: contractile force, contractile frequency, contractile duration and contractile stamina.

### EXAMPLES

Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Materials and Methods:

Generation of SHF-dsRed Transgenic Mice.

A 3.97 kb enhancer fragment from the 5′ regulatory region of murine Mef2C geme (Lien et al., 1999) (a kind gift from Dr. Brian Black, UCSF) was inserted into a promoterless dsRed expression vector (Invitrogen). The DNA insert, including the dsRed expression sequence, was introduced into the pronucleus from C57B1/6 mice (Charles River Laboratories, Wilmington, MA). Of the initial founders, one was further expanded. All animal experiments described in this paper have been approved by Animal Resources at Massachusetts General Hospital, MA.

Generation of SHF-dsRed/Nkx2.5-eGFP ES Cell Lines.

Timed matings were performed between SHF-dsRed transgenic males and Nkx2.5-eGFP females. On day 3.5 PC, the females were sacked and the blastocysts flushed from the uterine horns using M2 medium (Sigma-Aldrich, MO). After washing with M2 media, the zona pellucida was removed with acidic Tyrode's Solution (Sigma-Aldrich, MO) and the blastocysts were further washed three times in M2 media. The blastocysts were then adapted onto mouse embryonic feeder cells (MEF) with derivation media (DMEM with 15% KOSR, pen/strep, pyruvate, nonessential amino acids, and leukemia inhibitory factor [LIF] [Chemicon, CA]).

In Vitro Differentiation of ES Cell-Derived SHF-dsRedNkx2.5+ Cells.

ES cells were cultured and adapted to gelatin-coated dishes in the presence of leukemia inhibitory factor for 2 days prior to differentiation. ES cell differentiation was performed according to a previous published protocol (43). On the day of sorting, EBs were digested with either trypsin/EDTA for 5 min. These cells were then resuspended in PBS with 10% FCS and analyzed on a FACSAria. This treatment protocol resulted in greater than 90% live single cells.

Isolation of Embryonic Cardiac Progenitors.

Embryos from timed matings of Nkx2.5-eGFP/SHF-dsRed double transgenic mice were dissected. A single-cell suspension was obtained by gentle trypsinization followed by passing through a 40 μM cell strainer. eGFP+/dsRed+ (R+G+), eGFP+ (R− or R−G+), dsRed+ (R+G−), and double negative controls (R−G−) were isolated by FACS on FACSAria (BD Biosciences) and lysed with TRIZOL® reagent (Invitrogen, Carlsbad, Calif.) or cultured in differentiation medium (DM) containing IMDM with high glucose, 20% FCS, 5000 i.u./mL penicillin/ streptomycin, 200 mM L-glutamine , 1-thioglycerol (1.5×10-4M), and ascorbid acid (50 μg/mL). Flow cytometry data was processed using FLOWJO® v4.6.2 (Tree Star, Ashland, Oreg.) software.

RNA Isolation from Embryonic and ESC-Derived Cardiac Progenitors.

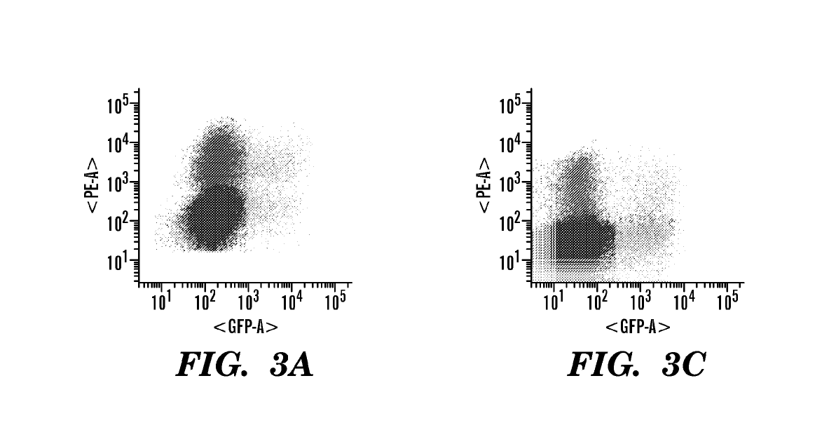

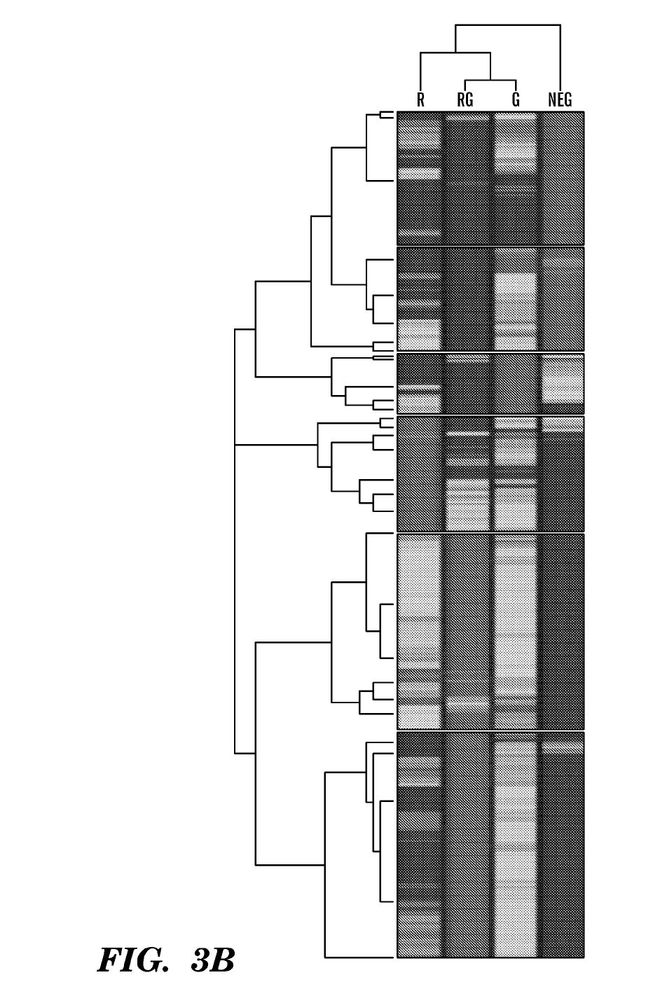

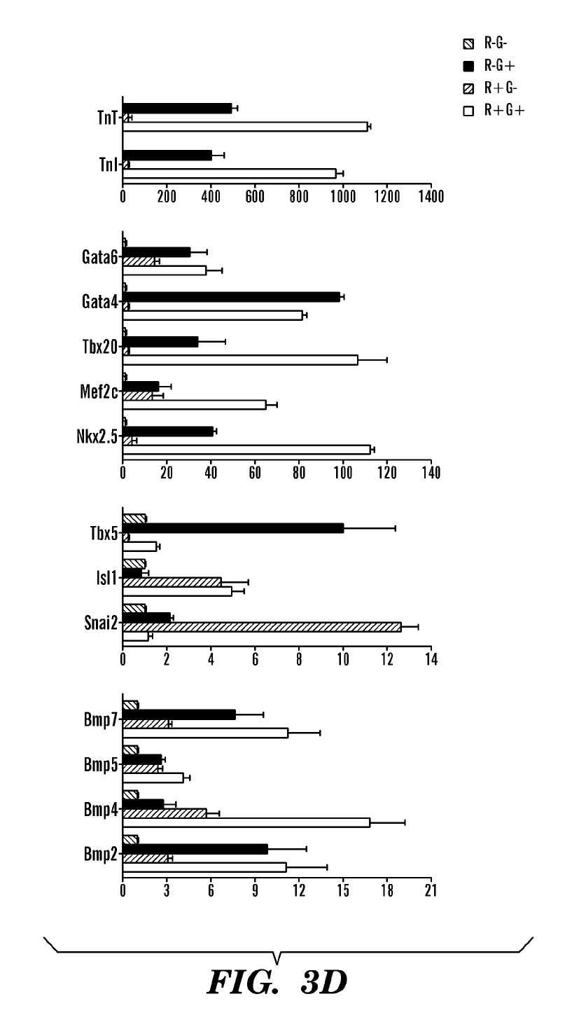

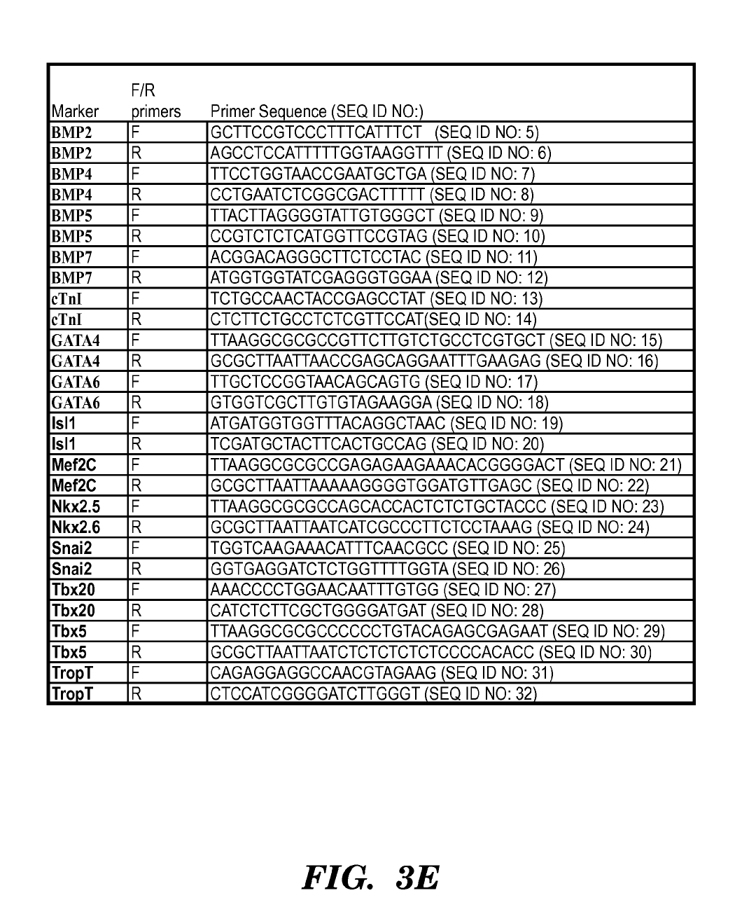

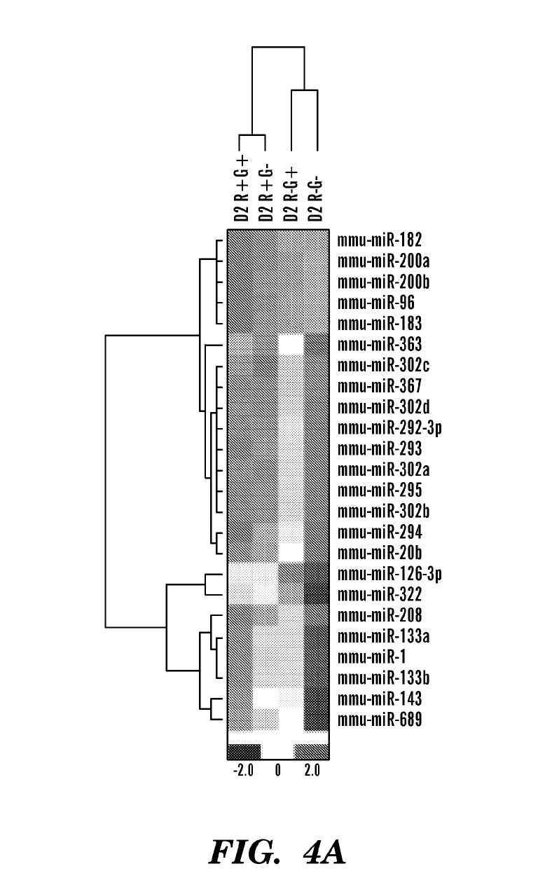

Sorted cells from embryonic hearts or in vitro differentiated ES cells were immediately added to TRIZOL® reagent (Invitrogen, Carlsbad, Calif.) and stored at −80° C. until processed. Total RNA from each sample was purified from cell lysate using the MIREASY RNA ISOLATION® (Promega, Madison, Wis.) according to manufacturer's suggested protocol. Qualitative and quantitative PCR were performed on cDNA made from reverse transcribed RNA using the I-SCRIPT® cDNA synthesis kit (BioRad, Hercules, Calif.) for total cell number >100,000, or CellsDirect cDNA synthesis system (Invitrogen, Carlsbad, Calif.) for cell number less than 100,000. Qualitative RT-PCR was performed using TAQ® polymerase (Roche Diagnostics, Indianapolis, Ind.) within the linear range of amplification (25-33 cycles) for each primer. Quantitative PCR was performed using the I-CYCLER® system with SYBR® Green substrate (BioRad, Hercules, Calif.) for 40 cycles. PCR Primers used in the qRT-PCR are shown in FIG. 3E.

Generation of Chimera Mouse Embryos Containing Nkx2.5-eGFP/SHF-dsRed Transgenic ES Cells.

Nkx2.5-eGFP/SHF-dsRed double transgenic ES cells were microinjected into E3.5 blastocysts from C57B1/6 females (8 cells/blastocyst) and implanted into the uteri of pseudopregnant CD-1 foster mothers. At E10.5 foster mothers were sacrificed and embryonic hearts visualized under whole mount fluorescence microscopy (Axiophot, Zeiss).

Immunofluorescence Studies.

Antibodies used in this study include: eGFP (BD, chicken anti-eGFP), dsRed (BD, rabbit polyclonal), Ki67 (BD, rabbit polyclonal), sarcomeric _-actinin (Sigma, mouse monocloncal), smooth muscle myosin heavy chain ((SM-MHC, 1:100) Sigma, rabbit polyclonal), and PECAM1 (sigma, rabbit polyclonal). Subsequently samples were incubated with the appropriate secondary antibodies conjugated with EITHER Alexa-Fluor 488 OR Alexa-Fluor 594 (Invitrogen) and mounted with DAPI (Invitrogen). Quantification of differentiation potential of ES-derived cardiac prognitors on micropatterened substrate was performed by slide staining with anti- sarcomeric _-actinin and anti-SM-MHC antibodies and cell counting (performed in triplicate) or with PECAM1 (to evaluate endothelial differentiation).

Genome wide Transcriptional Profiling.

Microarray expression profiling on RNA isolated from 3 distinct populations of cardiac progenitors. The double transgenic ES cell line was allowed to differentiate in vitro and FACS sorting was performed on EB day 6 as described above. 1,000,000 cells were isolated from each of the 4 populations of cells. Experiments were repeated in biological triplicates for a total of 12 microarrays. Total RNA was arrayed on the Affymetrix 430.20 chip. The labeling, hybridization, and scanning of the microarray experiments were performed at the Dana Farber Cancer Institute Microarray Core Facility. Data analysis was performed on the GenePattern (44). Consensus clustering was performed using a hierarchical clustering algorithm (kₘₐₓ=5). For Hierarchical clustering of gene expression, data sets was preprocessed with the GenePattern Preprocess Dataset Module with parameters set for a minimal change of 2.5 fold and a minimum delta of 20. Hierarchical clustering was then performed using the GenePattern Hierarchical Clustering Module with a Pearson Correlation and pairwise linkage. The data was then displayed as a heat map with a tree structured dendrogram.

Genome wide miRNA Profiling and Validation.

5 ug of total RNA from each progenitor population was isolated and was hybridized to the Exiqon miRNA microarray. The RNA samples were analyzed for integrity on the BIOANALYSER2100 and RNA measurement was performed on the Nanodrop instrument. The samples were subsequently labeled using the miRCURY™ Hy3™/Hy5™ power labeling kit and hybridized on the miRCURYTM LNA Array (v.9.2) produced by Exiqon according to manufacturer's protocol. The samples were then hybridized on a hybridization station. A total of 4 microarrays were used, each comparing one of the samples to a pooled reference sample. Analysis of the scanned slides showed that the labeling was successful with all control probes producing signals in the expected range. The quantified signals (no background correction) were normalized using the global Lowess (LOcally WEighted Scatterplot Smoothing) regression algorithm, which produces the best within-slide normalization to minimize the intensity dependent differences between the dyes.

Clustering analysis was performed on log2(sample/control or “Hy3/Hy5”) ratios which passed the filtering criteria of >0.5 standard deviation on variation across populations. A heat map was then generated showing the result of one-way hierarchical clustering of miRNAs and progenitor populations.

To validate the developmental expression pattern of miRNAs real time PCR (qPCR) analysis using Taqman Assays (Applied Biosystems) was performed on total RNA isolated from FACS sorted purified embryonic progenitor populations. ED9.5 double transgenic embryos were dissociated into single cell suspension. FACS sorting of 4 populations of cells, NEG (R−G−), G+(R−G+ or eGFP), R+ (R+G− or dsRed), and R+G+ (or dsRed/eGFP) was performed in biological triplicate with an average of 50,000 cells isolated from approximately 100 double transgenic embryos per experiment as described above. Taqman Assays (Applied Biosystems) were performed on total RNA isolated from FACS sorted purified embryonic progenitor populations. Expression was normalized to the sno410 small nuclear RNA and the double negative population was used as a calibrator and set as 1. A subset of differentially expressed miRNAs is displayed.

Statistical Analysis.

qPCR data is presented as mean ±SD. Differences between groups were compared with ANOVA with Bonferroni post-hoc analysis. For the dichotomous variables, the inventors used the Fisher-exact test. p-values below 0.05 were considered statistically significant. For all statistical analysis SPSS version 13.0 was used.

Tissue Engineering to Generate 2-Dimensional Ventricular Myocardium from ESC Derived Progenitors.

In the absence of extracellular gradients (chemical, mechanical, electrical or physical), cardiomyocytes cultured in vitro self-assembled with no preferential alignment of cell bodies and thus no net direction of contractile stress or strain. However, when cardiomyocytes were cultured on 20 μm wide, alternating fibronectin and Pluronic F127 lines, the cells spontaneously aligned, similar to published results (E. Dodou, S. M. Xu, B. L. Black, Mech Dev 120, 1021 (2003)). This engineered anisotropic 2D myocardium had uniaxial sarcomere alignment indicating a contractile direction along the length-wise axis of the cardiomyocytes and was scalable to the mm length scale. This produced an array of discrete muscle fibers with uniaxial alignment (see 6D and 9A, 9B and data not shown). Contraction of an entire MTF occurred spontaneously or when using external field stimulation electrodes and a voltage sufficient to activate a substantial number of fibers. In total, by engineering surface chemistry and providing geometric cues encoded in the extracellular matrix, we controlled tissue microstructure to 2-D anisotropic ventricular tissue from a renewable ESC based cell source. Of great importance is the discovery that the engineering of force generating contractile 2-D myocardial tissue was only possible from the R+G+ (dsRed/eGFP) progenitor population and not from the R+G− (dsRed) or the R−G+ (eGFP) progenitor populations. When the latter were grown on the engineered surface chemistry, an insufficient number of cells differentiated into functional myocardial tissue. As a result, tissue derived from these progenitors populations was unable to contract synchronously and bend the PDMS film of the MTF. This finding highlights the importance of isolating homogenous and highly purified populations of committed ventricular progenitors for the generation of tissue engineered myocardial tissue for functional analysis.

Surface Fabrication for 2-Dimensional Engineered Myocardium

PDMS thin film substrates were fabricated via a multi-step spin coating process, based on established methods (E. Dodou, S. M. Xu, B. L. Black, Mech Dev 120, 1021, 2003 and W. Feinberg et al., Science 317, 1366, 2007). Glass cover slips (25 mm diameter) were cleaned by sonicating for 60 minutes in 95% ethanol and air dried. Next, poly(N-isopropylacrylamide) (PIPAAm, Polysciences) was dissolved at 10 wt % in 99.4% 1-butanol (w/v) and spin coated onto the glass cover slips for 1 minute at 6,000 RPM. Sylgard 184 (Dow Corning) polydimethylsiloxane (PDMS) elastomer was mixed at a 10:1 base to curing agent ratio and spin coated on top of the PIPAAm coated glass cover slip and cured at 65° C. for 4 hours. For immunohistochemistry experiments, the cover slips were spin coated with polydimethylsiloxane (PDMS) elastomer (Sylgard 184, Dow Corning) without the PIPAAm layer.

Microcontact printing of FN was used to align the progenitors and cardiomyocytes and achieve an anisotropic pattern of 2-dimensional myocardium. Based on established methods (E. Dodou, S. M. Xu, B. L. Black, Mech Dev 120, 1021, 2003 and W. Feinberg et al., Science 317, 1366, 2007), PDMS stamps were fabricated with 20 μm wide, 2 μm tall ridges, separated by 20 μm spacing. Stamps were cleaned with 50% ethanol, air dried and incubated with FN in DI water (50 μg/mL) for 1 hour. The stamps were then rinsed twice in DI water, dried with compressed air and stamped on the PDMS coated cover slip or on the MTF. After stamping, the surfaces were incubated with 1% Pluronic F127 (BASF Group) solution for 5 min and then washed 3 times with phosphate buffered saline prior to cell seeding.

Muscular Thin Films of ES-derived Progenitors.

FACS-purified ES derived eGFP+/dsRed+cells were differentiated for five days on micropatterned PDMS coverslips (seeding density of 4×10E⁵/cm²), trypsinized and reseeded on MTFs. These progenitor derived cardiomyocytes were cultured for two days on the MTF, required to allow cells to settle out of suspension, adhere to the MTF and reform cell-cell contacts. Cover slips were then removed from the incubator and transferred to a Petri-dish filled with normal Tyrode's solution at 37° C. Under stereo dissection, a 2 mm by 5 mm rectangular MTF was cut out with cardiomyocytes longitudinally oriented. In some instances, MTFs were 1 cm long and 3 mm wide with anisotropic cells longitudinally oriented. The Tyrode's temperature was decreased to room temperature in order to dissolve the underlying PIPAAm, releasing the MTF from the cover slip. The MTF was then anchored to a small holder and viewed side-on under field stimulation (10V, 10 ms pulse-width) at a pacing rate of 0.5 Hz. High-speed video recording of the MTF was post-processed using MATLAB-based image analysis software to determine the change in radius of curvature as a function of time. Peak systolic stress generated by the cardiomyocytes along the longitudinal axis of the MTF was calculated using a modified Stoney's equation (16). Further, the constant curvature indicates that the cardiac tissue generated a constant stress throughout the progenitor-derived myocardial tissue. This demonstrates that cardiomyocytes were uniformly differentiated and verifies our capability to tissue-engineer functional, anisotropic myocardium from a renewable cell source.

Data Capture and Image Analysis

Experiments on live MTF constructs were conducted at room temperature (˜22° C.) in Tyrode's solution (exchanged every 30 minutes). All data was recorded within 2 hours following preparation. Video microscopy of MTFs was accomplished with a stereomicroscope coupled to a Sony DCS-V3 digital camera to record video (640×480 pixels, 25 fps). External pacing used parallel platinum wire electrodes spaced ˜1 cm apart and lowered directly into the Petri dish containing ˜8 mL normal Tyrode's solution. The voltage required to capture MTF contraction varied from 5 to 7 volts. To ensure capture, an external field stimulator (Myopacer, IonOptix Corp.) was used to apply a 10 V, 10 msec duration square wave at pacing rates of 0.5 and 1 Hz for durations of up to 2 minutes. Analysis of MTF motion was performed in a post-processing step by tracking the frame-to-frame displacement with image processing software. Video clips were converted from MPEG to uncompressed AVI and opened in ImageJ (National Institutes of Health) as image stacks. The conversion factor from pixels to micrometers was calculated for each video clip using the millimeter ruler included in the field of view for calibration.

Electrophysiology.

FACS-purified ES derived cells were cultured for 5 days. Patch electrodes were filled with an intracellular solution containing 140 mM potassium gluconate, 10 mM NaCl, 2 mM MgCl₂, 10 mM HEPES, 1 mM EGTA, 4 mM Mg-ATP, and 0.3 mM Na-GTP at pH 7.3, giving resistances of 2-5 MΩ. Perfusion with TTX was performed with a constant perfusion catheter and 20 μM solution of tetrodotoxin (TTX); wash out was performed with perfusion buffer. Spontaneous cardiomyocyte action potentials were recorded at room temperature using the whole-cell patch clamp method in current clamp mode with an Axopatch 200A amplifier (Axon Instruments/Molecular Devices, Sunnyvale, Calif.). Recorded data were filtered online at 1 kHz, sampled, and digitized (pClamp 9.2 software; Axon Instruments/Molecular Devices, Sunnyvale, Calif.).

### Example 1

Mammalian cardiogenesis requires the generation of a highly diversified set of both muscle and non-muscle heart cell lineages, including atrial and ventricular cardiomyocytes, conduction system and pacemaker cells, smooth muscle, endothelial, valvular, and endocardial cell types (For review, see (1-5). The formation of these various cardiovascular cell lineages in distinct heart and vascular compartments is based on the existence of a closely related set of multipotent progenitors in the early embryonic heart field (6-10) which can be divided into first (FHF) and secondary heart field (SHF) lineages (2, 11, 12). The secondary heart field lineages are marked by the expression of Islet-1, which give rise to most of the muscle and vascular cells in the heart itself, with the exception of the left ventricular chamber (6-8, 13), as well as contributing to epicardial lineages that play a critical role in coronary arteriogenesis (14, 15). In vivo lineage tracing and clonal cell assays have recently shown that Islet-1 multipotent progenitors which co-express Nkx2.5 can undergo self renewal and can give rise to a variety of cardiac tissues, including cardiomyocytes, smooth muscle, pacemaker and conduction system, and endothelial cells(7-9). However, it is still unclear as to the precise mechanism that governs the generation of large numbers of specific mature, differentiated cell progeny from these multipotent Islet-1 progenitors. The process could represent a stochastic event, sequential restriction to intermediates of more limited potency, directed differentiation from local cues, or the appearance of a committed, renewable subset of downstream progenitors in the Islet-1 lineage pathway that only make a specific fully differentiated cell type. Uncovering this pathway is a central question in cardiogenesis and has direct implications for cardiovascular regenerative medicine. In this regard, the inability to direct the differentiation of multipotent progenitors specifically to mature ventricular muscle remains a major obstacle for optimal in vivo cardiac myogenesis during cardiac repair following injury.

Herein, the inventors have developed an in vivo two-color reporter system to isolate first heart field (FHF) and secondary heart field (SHF) progenitors from mouse murine embryos and embryonic stem cells. Genome wide profiling of coding and non-coding transcripts revealed distinct molecular signatures of these progenitor populations. Thus the inventors have discovered that there are readily distinguishable signatures for the FHF and SHF progenitor subsets and that they represent distinct lineages. The inventors further identified a committed ventricular progenitor (CVP) cell in the Islet 1+SHF lineage that is capable of in vitro expansion, differentiation, and assembly into functional ventricular muscle tissue, representing a combination of tissue-engineering with stem cell biology.

As disclosed herein, by using a combination of positive and negative sorting for the two color reporters, the inventors have identified a subset of the Islet-1 lineage representing entirely committed ventricular progenitors (CVPs). These CVPs expand in culture and assemble into fully mature, rod shaped ventricular muscle cells, as assessed by single cell electrophysiological measurements.

Furthermore, a thin biological film seeded with a patterned monolayer of CVPs generates fully functional ventricular muscle tissue that has the ability to generate force, tension, and contractility that is quantitatively similar to biological thin films constructed from neonatal ventricular muscle cells (16). Accordingly, the inventors have demonstrated the formation of ventricular muscle is driven via a fully committed subset of ventricular cardiomyogenic progenitors that is capable of self-expansion and self-assembly. The ability to isolate these CVPs from ES cells to create functional ventricular muscle tissue may have widespread implications for regenerative cardiovascular medicine and drug discovery.

Recent work has identified an isll-dependent, secondary heart field (SHF) (or anterior heart field (AHF) specific enhancer element of the myogenic transcription factor Mef2c, which has been used to drive the expression of various reporters in transgenic mice (8, 17, 18). This enhancer element contains essential isll binding sites and is expressed in a subset of mesoderm lineages, specifically in the Right Ventricle (RV) and the Outflow Tract (OFT) as well as the pharyngeal mesoderm, a population of cells which will contribute to the majority of the cells of the RV and OFT(19-22). Significantly, it is not expressed in the FHF progenitors of the Left Ventricle (LV) and the inflow tract (IT).

The inventors, by using a SHF-specific Mef2c enhancer in combination with the pan-cardiac Nkx2.5 enhancer (9, 23), have discovered a way to uniquely label and purify and isolate distinct cardiac progenitor populations representing the primary and secondary heart fields at different stages of commitment.

### Example 2

Generation of Nkx2.5-eGFP and SHF-dsRed Transgenic Mice

Next the inventors generated a novel secondary heart field (SHF)-dsRed transgenic mouse line, with the red florescent protein dsRed under the transcriptional control of an Islet-dependent enhancer of the Mef2c gene whose expression is restricted to the SHF (the SHF-Mef2c enhancer) (18). The red florescent protein dsRed was downstream of the SHF enhancer.

A 6.1 kb cardiac specific enhancer fragment was inserted in a promotorless dsRed expression vector. The DNA insert was introduced in the pronucleus from wild-type mice. The DNA fragment containing both components was gel isolated and was used for pronuclear injection. The transgenic embryo was then implanted into pseudopregnant females and allowed to develop into mature animals. These animals were then crossed with wildtype females and the embryos were examined at ED9.5 of development. This allowed the identification of a transgenic mouse line with a dsRed expression pattern that was completely restricted to the SHF and its derivatives. The generated transgenic mice expressed dsRed specifically in the secondary heart field including the pharyngeal mesoderm the right ventricle (RV) and right ventricular outflowtract (RVOT).

The inventors bred this mouse line with the transgenic mouse line in which eGFP expression is under the control of the cardiac specific Nkx2.5 enhancer element (9, 23). This enhancer is expressed throughout the developing heart tube on embryonic days 8-10, but is not expressed in the pharyngeal mesoderm, residence of more primitive cardiac progenitors (Wu et al. Cell 2006).

By fluorescence microscopy of double transgenic embryos on embryonic day (ED) 9.5, the entire primitive heart tube was eGFP+, but only the right ventricle (RV) and the outflow tract (OFT) were also dsRed+. Further, the pharyngeal mesoderm (PM) which contributes to the RV and OFT was dsRed+ but eGFP− (data not shown). To delineate the in vivo expression of the reporters, the inventors performed immunohistochemistry on ED9.5 embryos and found that dsRed+/eGFP+cells (R+G+) were restricted to the RV and OFT, dsRed−/eGFP+ cells (R−G+) to the left ventricle (LV) and inflow tract (IFT), and dsRed+ cells (R+G−) to the pharyngeal mesoderm (data not shown).

Accordingly, by crossing these mice, the inventors developed a two-color system allowing the identification and isolation of different cardiac progenitor cell (CPC) populations: e.g. single GFP labeled cells (R−G+) (inflow-tract and left ventricular/CPC), single dsRed cells (R+G−) primitive pharyngeal mesoderm/CPC) and double labeled cells (GFP and dsRed or R+G+) (right ventricle and right ventricular outflow tract CPC). Accordingly, this unique combination allowed for the identification of different cardiac progenitor cell (CPC) populations and represented a fundamental advance in the ability of the inventors to isolate lineage restricted cardiac progenitors.

Embryonic stem cell lines (ESC) utilize many of the in vivo developmental programs, providing an attractive model system for lineage commitment. The inventors therefore generated multiple ESC lines that harbor both the Nkx2.5-eGFP and the SHF-dsRed reporters (data not shown). Fluorescence microscopy of chimeric embryos from these ESC lines revealed faithful recapitulation of marker expression (data not shown). In vitro differentiation by embryoid body (EB) formation resulted in discrete populations of R+G+, R+G−, and R−G+ cells by EB day 6. In particular, the inventors discovered using fluorescence microscopy of double transgenic embryos at ED9.5 embryos (looping heart tube), that the entire primitive heart tube (including both primitive ventricular chambers, the inflow-tract, and the outflow-tract) were marked with eGFP (R−G+), but only the RV and the OFT were also marked with dsRed (R+G−). In addition, the pharyngeal mesoderm (PM) was marked by only dsRed and not by eGFP (R+G−) (data not shown) and will contribute the majority of the cells of the RV and outflow tracts (Dodou et al., 2004; Verzi et al., 2005) was marked by dsRed but not eGFP (R+G−).

Cardiac progenitor cells represent a sub-population of the total ES cells and must be isolated in order to use them. Methods for cell isolation remain a critical technical issue with a variety of potential solutions. Accordingly, in order to further delineate the pattern of expression of eGFP and dsRed and in order to define the population of cells in embryos that are labeled by these markers, the inventors performed immunohistochemistry on double transgenic developing embryos. This confirmed the above-described pattern of marker expression with cellular resolution. Thus, using this 2 color system and fluorescently activated cell sorting (FACS) sorting, the inventors were able to uniquely identify and isolate three distinct populations of cardiac progenitors: <1> double labeled dsRed +/eGFP+(R+G+) population representing RV and outflow tract progenitors, <2> single labeled dsRed+ (R+G−) population representing primitive isl1+ pharyngeal mesoderm progenitors, <3> and single labeled eGFP+ (R−G+) population representing the LV and inflow tract progenitors. Comparisons were made pair wise across these samples and to the reference non-cardiac population which expressed neither dsRed nor eGFP (R−G−).

Accordingly, using this two-colored reporter system, the inventors were able to isolate distinguish between populations of LV and RV myocardial progenitor cells derived from the primary and secondary heart field at different stages of commitment (data not shown).

### Example 2

Generation of Nkx2.5-eGFP and SHF-dsRed Transgenic Mouse Embryonic Stem Cell Lines:

Although clonal studies have suggested the possibility of a common upstream precursor for the left and right ventricular precursors, the inability to isolate large amounts of purified, committed primary and secondary heart field progenitors has precluded their direct comparison.

Embryonic stem cell lines (ESC) can differentiate into many different cell lineages in vitro and utilize many of the in vivo developmental programs, providing an attractive model system for studying lineage commitment. For example, in vivo ESCs can contribute to all cell types of chimera mice; in vitro ESCs can differentiate through the formation of embryoid bodies (EBs) into a diverse set of cell populations with cell types from all three germ layers. Significantly, ESC in vitro differentiation can be scaled up to generate large numbers of cardiac progenitors. Therefore, the inventors generated multiple ESC lines that harbor both the Nkx2.5-eGFP and the SHF-dsRed reporters (data not shown).

In order to generate mouse ES cell lines that harbor both the Nkx2.5-eGFP and the SHF-dsRed markers, the inventors interbred these mouse lines and isolated blastocysts at ED3.5. After culturing in vitro on irradiated mouse embryonic fibroblasts (MEFs) in the presence of the Leukemia Inhibitory Factor (LIF), the inventors were able to generate ES cell lines that were derived from 3.5 day-old blastocyst-stage SHF-dsRed/Nkx2.5-eGFP mouse embryos that contained both markers. Blastocysts were collected and plated individually on a 24-well dish covered with irradiated mouse embryonic fibroblast (MEF) feeder monolayer, obtained from 14.5pci. mouse embryos to prevent differentiation. Germline transmission was tested by injection of these ES-cells into host blastocysts and implantation of these chymeric blastocysts into pseudo pregnant foster mothers. A chimeric double labeled heart was observed, indicating that the derived ES cells precisely recapitulate the expression pattern of the normal developing embryo. These ES cells were then allowed to differentiate in vitro. The double labeled SHF-dsRed/Nkx2.5-eGFP ES cells were maintained on irradiated MEFs and are grown in presence of leukemia inhibitory factor (LIF) in order to maintain an undifferentiated pluri-potent state.

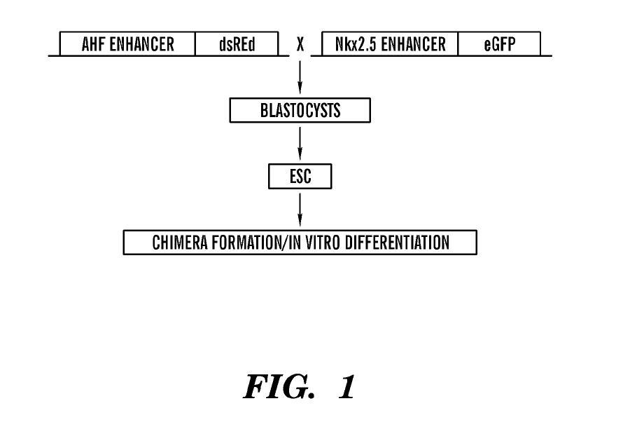

A hallmark of an ES cell is that it is capable of contributing to all the tissue of a developing embryo. To test the ability of these new ES cell lines to contribute to cardiac tissue, the inventors injected the ES cell lines into wildtype blastocysts, and the blastocysts were implanted into pseudopregnant females and allowed to develop until ED9.5. Florescence microscopy of the chimera embryos revealed ES cell contribution to primary and secondary derived cardiac structures with faithful recapitulation of dsRed and eGFP expression as described above (FIG. 1). This validated the mouse ES cell lines that the inventors had generated and justified their in vitro use as a surrogate for in vivo cardiogenesis. In vitro differentiation by embryoid body (EB) formation resulted in discrete populations of R+G+, R+G−, and R−G+cells by EB day 6 (data not shown).

Utilizing this novel ES cell line for in vitro differentiation assays and immunofluorescence microscopy, discrete populations of eGFP+ cells (R−G+), dsRed+ cells (R+G−), as well as eGFP+/dsRed+ (R+G+) cells were clearly evident by EB day 6 (FIG. 1) and by EB day 10 had formed beating clusters. The double-labeled transgenic ESC lines were differentiated in vitro for 6 days, at which point the EBs were dissociated into single cell suspension and FACS sorted. As in the case of the transgenic embryos, FACS analysis of ES cells differentiating in vitro revealed the presence of 3 distinct populations of cardiac progenitors: <1> double-labeled dsRed+/eGFP+ population (R+G+) (RV and OFT), <2> single-labeled dsRed+ (R+G−) population (PM), <3> and single-labeled eGFP+ (R−G+) population (LV and inflow tract). These cardiac progenitor populations were compared to the unlabeled (negative) (R−G−), non-cardiac population.

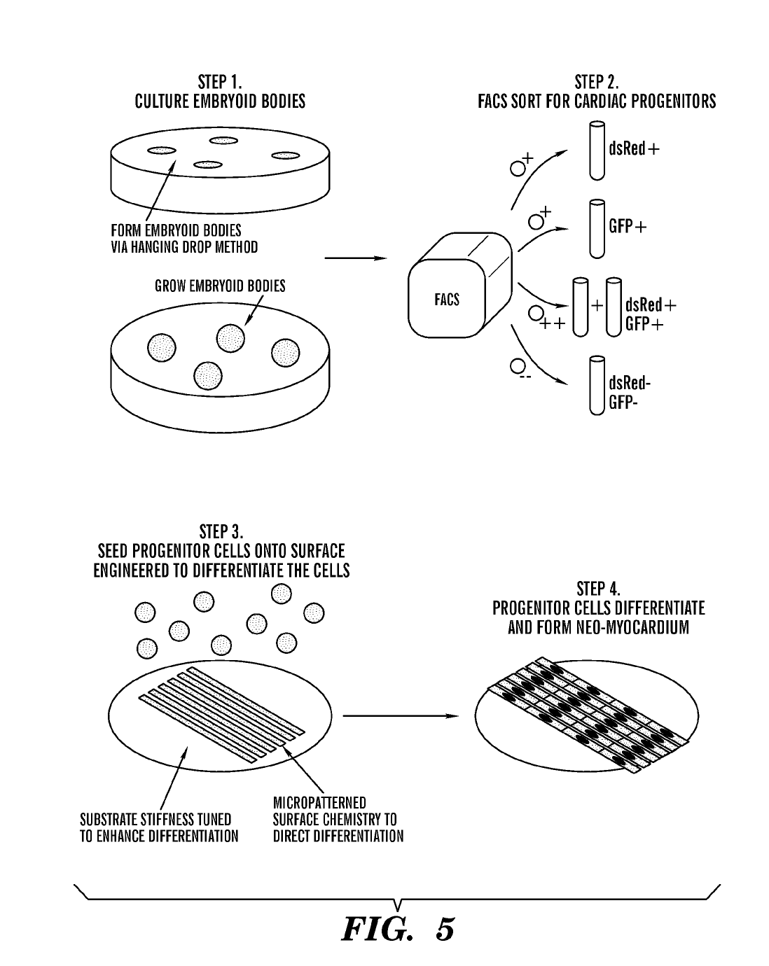

In order to promote in vitro differentiation., ES cells were adapted on gelatinized plates and two days later were allowed to differentiated in vitro by withdrawing LIF and allowing formation of embryoid bodies (EBs) by hanging drops. Six day EBs were dissociated into single cells with 0.25% trypsin. Cells were FACS sorted based on their ds-Red and eGFP expression (see FIGS. 5 and 6).

### Example 3

Identification of Myogenic Cardiac Progenitors:

As predicted, FACS analysis of ES cells differentiating in vitro revealed the presence of 3 distinct populations of cardiac progenitors as described above: (i) R+G+, (ii) R+G−, (iii) R−G+. These cell populations were FACS sorted on EB 6 and compared to the double negative or non-cardiac population (R−G−) as shown in FIG. 3D.

Real time PCR analysis of RNA isolated from FACS sorted cells revealed more than a 5 fold enrichment of the GFP transcript in the R+G+ and R−G+ populations. Likewise real time analysis also revealed nearly 20 fold enrichment of the dsRed transcript in the R+G+ and R+G− populations. These results provided important positive controls for the fidelity FACS sorting.

The inventors then examined the expression pattern of the cardiac transcription pattern isl1, nkx2.5, and mef2c in each of the single positive populations, the double positive population, as well as the double negative population. As expected both the nkx2.5 and mef2c demonstrated a significant enrichment in the R+G+, R+G−, and R−G+ populations compared to the double negative population. In contrast, isl1 which is the earliest marker of the secondary heart field was only enriched in the R+G− population. This population of cells represents the in vitro equivalent of the pharyngeal mesoderm and has been shown to have the highest levels of isll expression in the developing heart fields.

Cardiac Progenitor Differentiation:

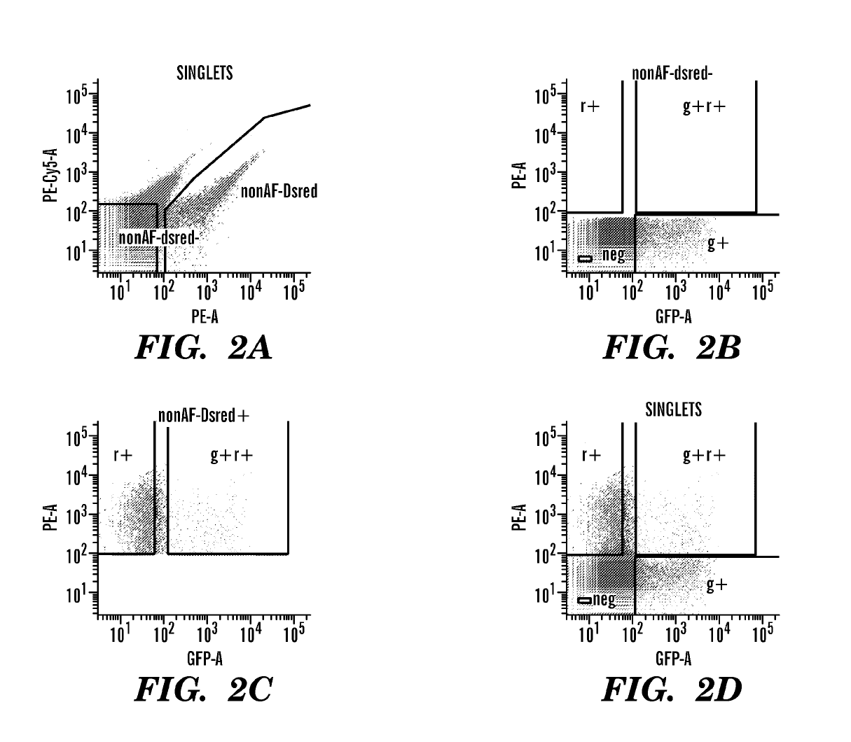

The different cardiac progenitor populations showed distinct differentiation patterns. FIG. 2A and 3D shows the cardiacmarker Troponin T (TnT) quantitative reverse transcriptase polymerase chain reaction (RT-PCR) for the different cardiac progenitors related to the negative population (=1).

In order to determine the developmental potential of the in vitro derived ES cells and ensure that they do recapitulate the in vivo developmental program, the inventors FACS sorted the 4 populations of cells and plated them onto fibronectin coated slides. Both the R−G+ and the R+G− populations (both secondary heart field progenitors) had the ability to spontaneously differentiate into beating cardiomyocytes and smooth muscle cells as demonstrated by immuno-staining for the cardiac specific marker Troponin T and the smooth muscle specific marker smooth muscle Myosin Heavy Chain (smMHC). The R−G+ population representing secondary heart field derived cells (the RV and outflow tract) had the ability to differentiate into beating cardiomyocytes but a diminished potential to differentiate into smooth muscle. This myogenic population of cells was used to generate contractile myocardial tissue on engineered myocardial tissue. This population of cells represents a novel population of cardiac progenitors with the capacity to undergo directed differentiation into cardiac myocytes. It therefore represents a fundamental advance in the field and gives us the opportunity to exploit this unique population for the study of cardiac lineage commitment, cardiac development, as well as drug identification and the study of drug toxicity.

### Example 4

Characterization and Marker Identification of the Isolated dsRed+/eGFP+ Cell Population:

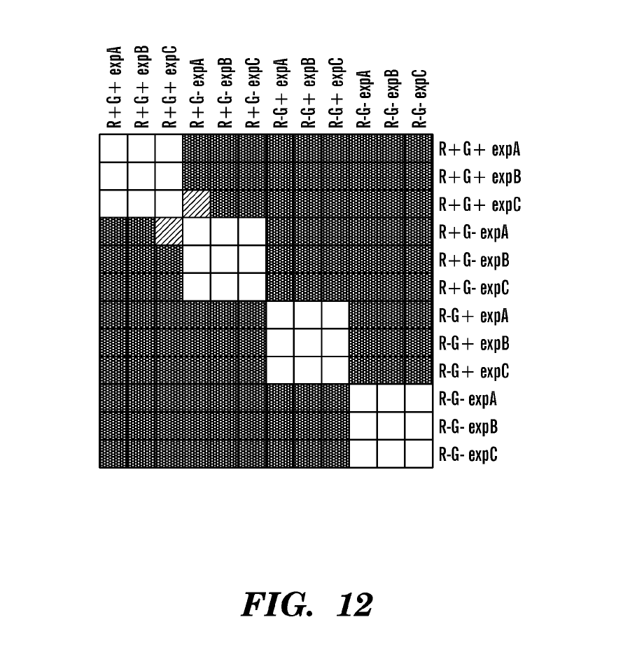

In order to perform comprehensive characterization of gene expression of primary and secondary heart fields progenitors at various stages of commitment, the inventors performed genome wide microarray expression profiling on RNA isolated from 3 distinct populations of cardiac progenitors. The double transgenic ES cell line was allowed to differentiate in vitro and FACS sorting was performed on EB day 6. 1,000,000 cells were isolated from each of the 4 populations of cells (eGFP+/dsRed+ (R+G+), e GFP+(R−G+), dsRed+(R+G−), and negative (R−G−)). The experiment was repeated in biological triplicates. Total RNA was arrayed on the Affymetrix 430.20 chip. Inter-experimental reproducibility and clustering stability was evaluated by performing consensus clustering on datasets from replicate experiments. This revealed that the genome wide transcriptional profile of each of the 4 populations of cells clustered together in replicate experiments, validating the experimental reproducibility (FIG. 12). In order to identify genes that were differentially expressed across the cardiac populations, hierarchical clustering was then performed to generate a tree structured dendrogram (FIG. 3B) showing clear distinct expression patterns for the different cardiac progenitor subsets.

To validate these distinct transcriptional expression profiles on embryonic cardiac progenitors, double transgenic ED9.5 embryos were dissociated into single cell suspension and FACS sorted. eGFP+/dsRed+ (R+G+), e GFP+ (R−G+), and dsRed+(R+G−) embryonic cells were compared to the unlabeled (negative) (R−G−) non-cardiac population representing the remainder of the embryo. All experiments were performed in biological triplicates or quadruplicates with each experiment constituting the RNA isolated from approximately 120 double transgenic mouse embryos (approximate 50,000-150,000 cardiac progenitor cells). The inventors validated a subset of genes identified by genome wide transcriptional profiling as being differentially expressed in the different cardiac progenitor populations by performing real time PCR analysis. These included both structural genes as well as transcriptional regulators. b-actin was used as an internal normalization control.

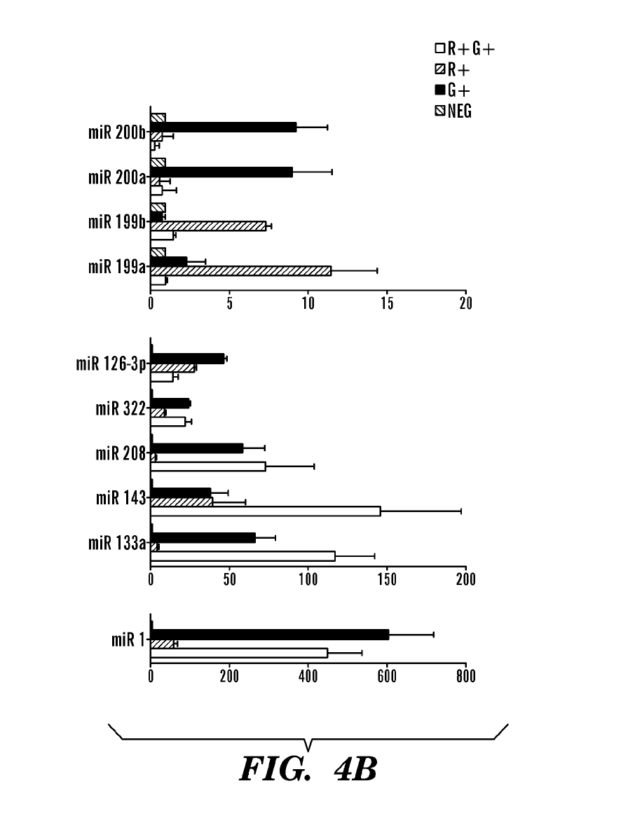

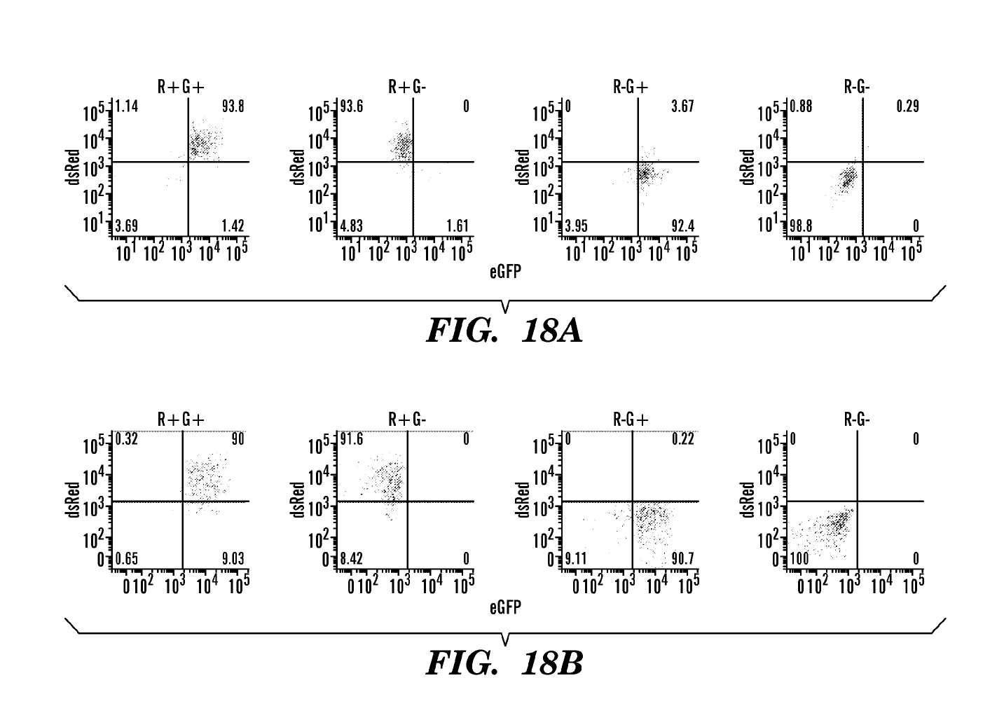

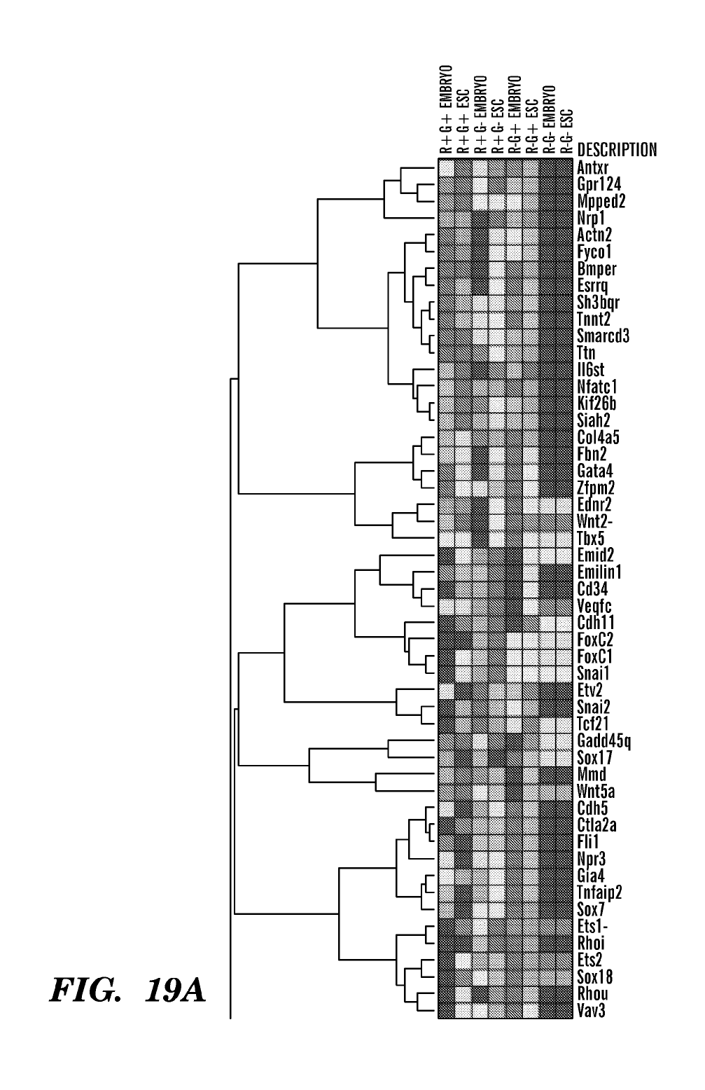

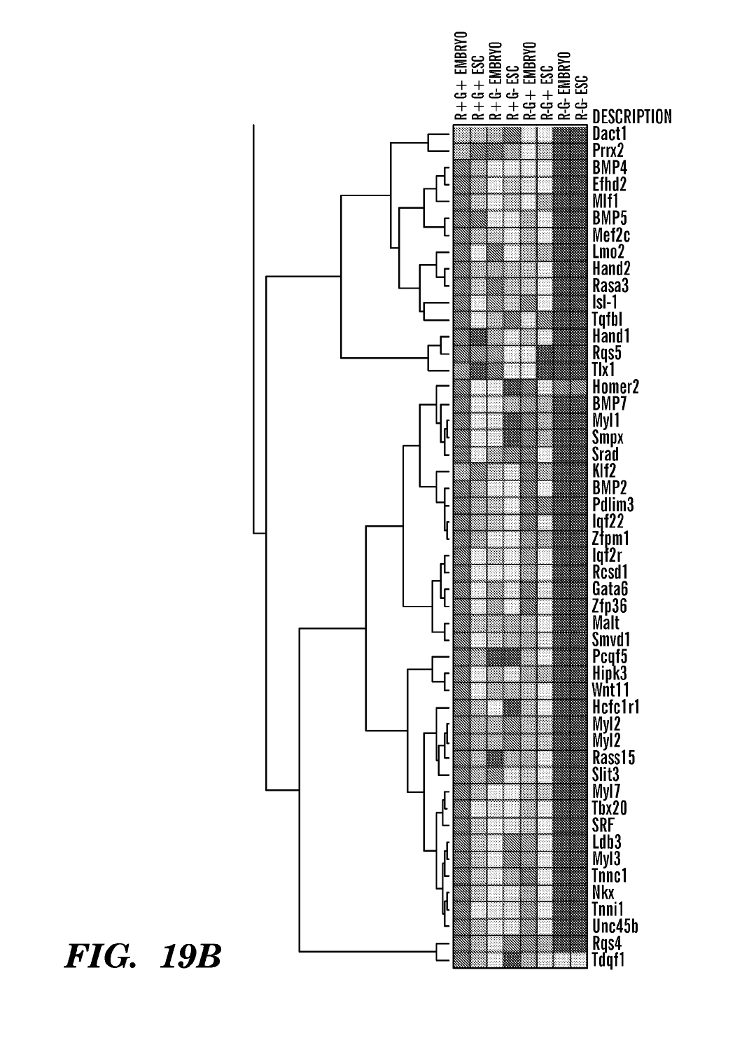

To isolate ESC-derived FHF and SHF progenitor cells, we dissociated day 6 EBs into single cell suspension and FACS-purified four distinct populations of cells: R+G+, R+G−, R−G+, and unlabeled (R−G−) (FIGS. 18A, 18B), and then performed DNA microarray analysis on coding and non-coding RNA. Hierarchical clustering (M. Reich et al., Nature Genetics 38, 500 (2006)) showed distinct reproducible expression patterns for the different cardiac progenitor subsets of mRNAs as well as microRNAs (miRNAs) (FIGS. 3B, 4B, 12, and the Table shown in FIG. 24). Next, the inventors FACS purified ED9.5 embryonic progenitors (FIG. 18A, 18B). Real time PCR (qPCR) analysis on 100 mRNAs and 10 miRNAs revealed that ESC and embryonic derived progenitors, isolated immediately after FACS sorting, displayed similar but non-identical patterns of expression (FIG. 19A, 19B). mRNAs and miRNAs implicated in cardiac development and disease were enriched in the colored cells compared with unlabeled cells. Isl1, a marker for the SHF was appropriately enriched only in the R+G+ and the R+G− populations whereas T-box transcription factor 5 (Tbx5), a marker of the FHF (Bruneau et al., Cell 106, 709, 2001; Mori et al., Dev Biol 297, 566, 2006), was appropriately enriched only in the R−G+population. The R+G+ cells appeared to resemble more closely the myogenic population based on the expression of myocardial markers such as cardiac troponins, cardiogenic transcription factors, and bone morphogenetic protein (BMP) signaling molecules. Further, the R+G− population of the PM expressed high levels of Snai2, a transcription factor regulating epithelial to mesenchymal transition (EMT) and necessary for cell-migration (Barrallo-Gimeno, et al., Development 132, 3151, 2005; Blanco et al., Development 134, 4073, 2007), demonstrating that SHF/PM progenitors undergo EMT prior to migrating during cardiogenesis. In addition, miRNA199a/b were preferentially expressed in the R+G− population and miRNA200a/b in the R−G+ population and may therefore be considered cardiac markers for the SHF and FHF, respectively (FIGS. 3D, 5B).

The inventors discovered that genes encoding contractile proteins as well as known cardiac transcription factors were enriched in the cardiac progenitor cell (CPC) populations compared to the double negative (R−G−) control. Of note, isll a marker for secondary heart field (SHF) progenitors, was appropriately enriched in the dsRed+ (R+G−) and the dsRed+/eGFP+(R+G+) population but not the eGFP+ (R−G+) populations. Furthermore, the dsRed+/eGFP+ (R+G+) appeared to be the most myogenic cell population as evident by the markedly increased level of expression of definitive myocardial markers such as Troponin T, Troponin C, as well as developmentally regulated cardiogenic transcription factor (such as Nkx2.5, Mef2c, Tbx20, GATA4, AND GATA 6) and BMP signaling molecules (FIG. 3C). Similarly the eGFP+(R−G+) population also demonstrated elevated levels of these structural and regulatory proteins, consistent with this population's myogenic potential. In contrast to the dsRed+/eGFP+ (R+G+) population, however, eGFP+ (R−G+) cells express Tbx5, a marker for primary heart field (FHF) progenitors, but not isll, a marker of secondary heart field (SHF) progenitors. Sorted cells from this two-colored system represent distinct FHF and SHF derivatives since they express either Tbx5 or Isl1 markers for FHF and SHF progenitors respectively (6, 13, 24, 25), in addition to their distinct and characteristic pattern of distribution with the dsRed+/eGFP+ (R+G+) cells anatomically located in the primitive RV and OFT whereas the single eGFP+ cells are located in the primitive LV (FIG. 3). Interestingly, the dsRed+ (R+G−) population of the pharyngeal mesoderm (PM) expressed high levels of Snai2, a transcription factor implicated in the regulation of the epithelial to mesenchymal transition (EMT) (26, 27). This demonstrates that secondary heart field (SHF) progenitors of the pharyngeal mesoderm (PM) to undergo the EMT prior to migrating into the developing heart to form the RV and outflow tract. Thus this two-color reporter system has allowed the inventors to unambiguously identify and isolate RV and LV myocardial progenitor cells at different stages of commitment.

In order to identify novel miRNAs involved in cardiac lineage specification, miRNA microarray experiments were performed with the miRCURY™ LNA Array (v.9.2). Hierarchical clustering revealed that the 3 different populations of cardiac progenitors had distinct patterns of miRNA expression. To validate the developmental expression pattern of miRNAs real time PCR analysis using Taqman Assays (Applied Biosystems) was performed on total RNA isolated from FACS sorted purified ED9.5 embryonic progenitors. miRNAs that were found to be differentially expressed in embryonic progenitors are shown in FIG. 5 The dsRed+/eGFP+ progenitor population expressed high levels of miRNAs known to play a role in cardiac development and disease. In addition, miRNA199a and miRNA199b were preferentially expressed in the dsRed+ population whereas miRNA200a and miRNA200b were expressed in the eGFP+ population and as such can be considered markers for the primary heart field.

### Example 5

Identification of a Fully Committed Ventricular Cardiac Progenitor Cell in the Islet-1 Lineage that is Capable of Limited Expansion and Spontaneous Self-Assembly Into Rod Shaped Ventricular Muscle Cells.

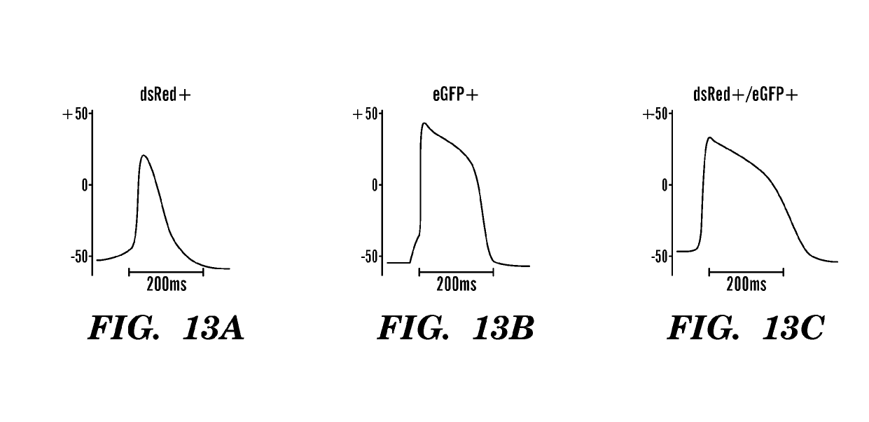

ED 9.5 hearts from double transgenic Nkx2.5-eGFP/SHF-dsRed mice were dissociated into single cell suspension and 2 color FACS sorting was performed. The four different populations were plated onto polydimehylsiloxane (PDMS) elastomer with microcontact-printed surfaces and were allowed to develop for an additional 3-5 days in vitro thereby constructing anisotropic cardiac tissue. Micropatterns of alternating 20 μm-wide lines of high density fibronectin lines and Pluronic F127 resulted in fibers of cells longitudinally aligned (FIG. 9A). Immunofluorescence with cardiac alpha-actinin and smooth muscle Myosin Heavy Chain (sm-MHC) antibodies demonstrated that the dsRed+/eGFP+ population gave rise to >95% cardiomyocytes whereas the dsRed+/eGFP−dsRed−/eGFP+ populations gave rise to a more heterogeneous population consisting of both smooth muscle and cardiomyocytes (FIG. 9), consistent with the transcriptional profile of these populations. In a similar manner, anisotropic cardiac tissue was generated from ES derived dsRed+/eGFP+ cardiac progenitors and showed almost exclusive cardiac myocyte commitment (FIG. 9B). To further specify the properties of the cardiomyocytes derived from the dsRed+/eGFP+ progenitors, we performed single cell patch clamping on anisotropic ESC-derived cardiac tissue. Analysis of 11/12 consecutive cells from the dsRed+/eGFP+ population revealed a mature ventricular-like action potential (FIG. 9C). The dsRed+ and the eGFP+ progenitor populations were not as myogenic with only a few of patch clamped cells showing a ventricular action potential (FIG. 13). These findings further reaffirm the distinctive ventricular myogenic properties of the dsRed+/eGFP+ cardiac progenitors.

A hallmark of progenitor cells is their capacity for cell-expansion in addition to differentiation. In order to evaluate this, Hoechst staining and FACS analysis were used to perform cell cycle analysis of undifferentiated ESC, EB day 6 cardiac progenitors, and their differentiated progeny (d6+5). Both undifferentiated ESC and EB day 6 cardiac progenitors had approximately 40-60% of cells in S or G2 phase but the differentiated progeny had less than <10% of cells in S or G2 phase (data not shown).

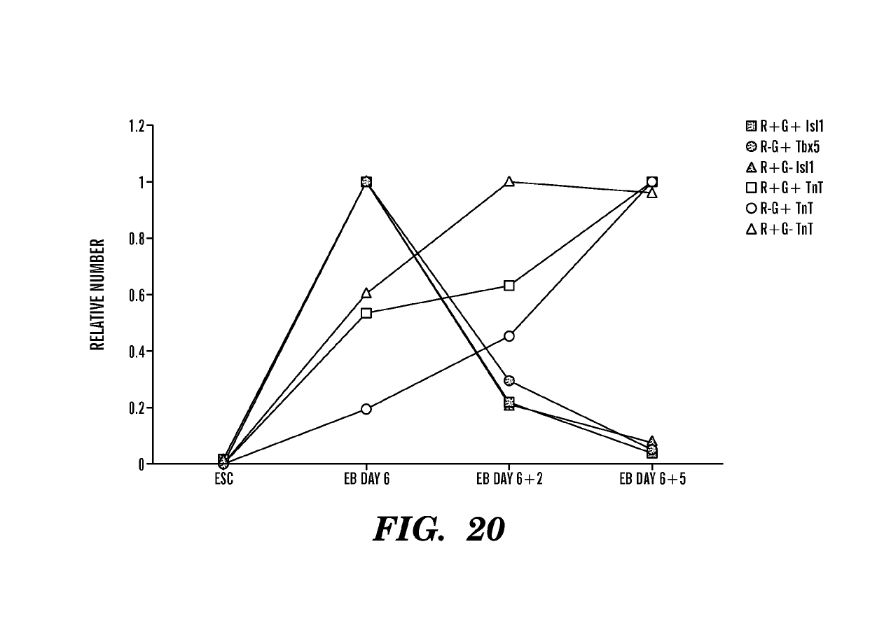

For validation, the inventors isolated EB day 6 progenitors and allowed them to expand in vitro for an additional 5 days. Immunostaining with Ki67, a marker for actively cycling cells, showed that 24 hours after isolation, most cells were actively cycling but this decreased over five days. Conversely, total cell number increased by four-fold (Figure data not shown). Furthermore, the expression of the progenitor markers Isl1 or TbxS was maximal at the time of progenitor isolation but decreased with further differentiation (FIG. 20). In contrast Troponin T expression continued to increase with differentiation (FIG. 20). Thus, the progenitor populations have a real but limited in vitro expansion potential. The drop off in expansion is concomitant with differentiation and loss of progenitor marker expression, demonstrating that an endogenous clock may limit their proliferative capacity.

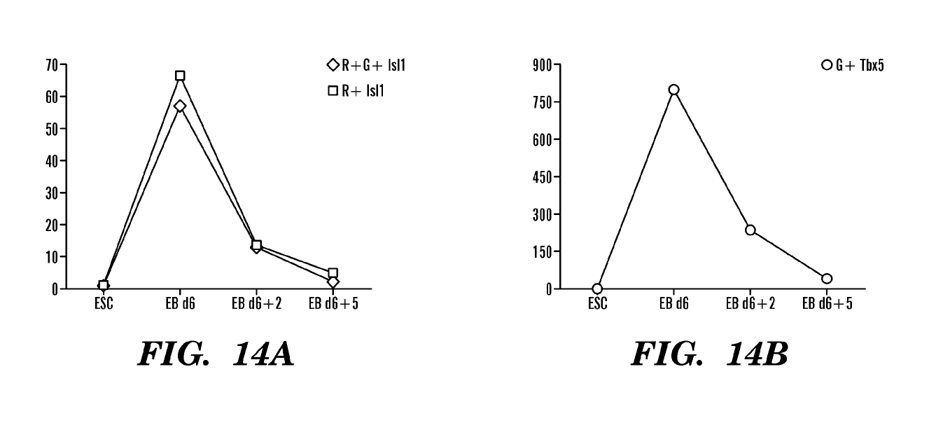

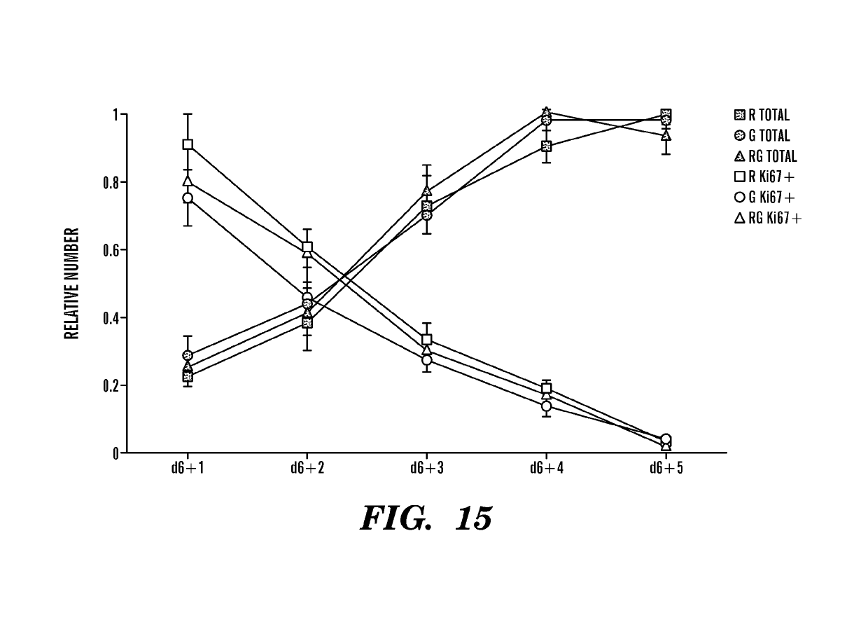

Secondary heart field (SHF) progenitors express Isl1 and this expression decreases with development both in vivo and in vitro (6, 13, 21). In order to evaluate the expression level of isll during the development of the dsRed+/eGFP+(R+G+) and dsRed+ (R+G−) cardiac progenitors, the inventors compared isll levels at EB day 6 and after a further 2-5 days of in vitro differentiation. As shown is FIGS. 14A and 14B, isll is expressed at peak levels on EB day 6 and this wanes with further expansion and differentiation such that it is turned off completely by day 11 of differentiation. In a parallel manner, TbxS (a marker for first heart field progenitors (24, 28)) is expressed at peak levels on EBD 6 of eGFP+ primary heart field progenitors, and its expression also wanes with further differentiation. These findings demonstrate that the identified cardiac progenitor populations expand prior to differentiation and are consistent with the in vivo developmental program where early progenitors can divide during the embryonic phase but that this capacity decreases during development such that mature ventricular myocytes essentially have no capacity for cell division or expansion (29).

### Example 6

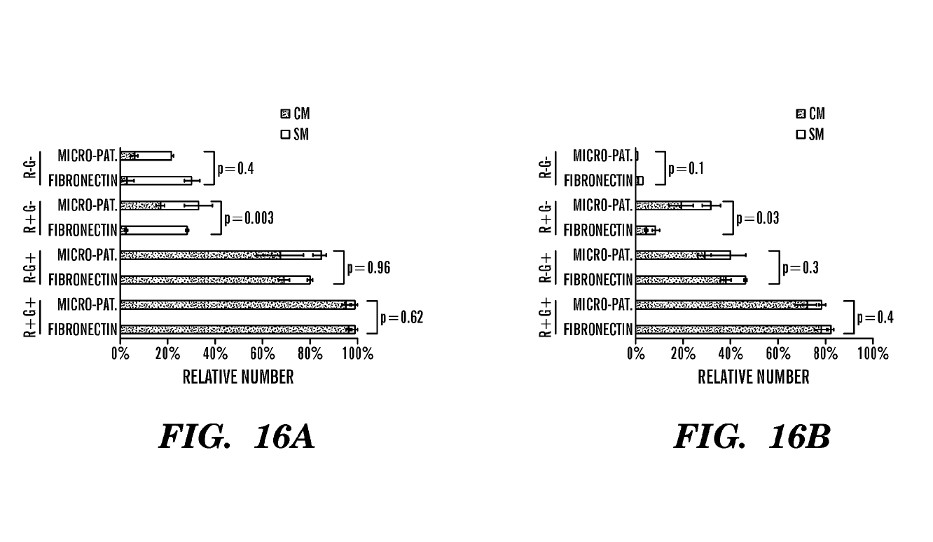

To examine progenitor myogenic potential, the inventors cultured embryonic and ESC-derived progenitors on either fibronectin coated slides or micropatterns of 20 μm wide lines of fibronectin alternating with 20 μm wide lines of Pluronic F127 (a surfactant that blocks cell adhesion). After 5 days of in vitro expansion and differentiation, the inventors performed immunofluorescence staining for sarcomeric-actinin and smooth muscle Myosin Heavy Chain (sm-MHC), labeling cardiomyocytes and smooth muscle, respectively. Plating embryonic and ESC-derived R+G+ cells on micropatterned surfaces resulted in anisotropic tissue consisting of longitudinally aligned myocardial fibers (FIGS. 6D and 9A, and data not shown). In contrast, plating the progenitor populations on un-patterned slides resulted in isotropic unaligned tissue (data not shown). Cell counting showed that embryonic and ESC derived R+G+progenitors primarily gave rise to cardiomyocytes independent of surface culture conditions. In contrast, the R+G− and the R−G+ populations gave rise to a more heterogeneous population of both smooth muscle and cardiomyocytes (FIGS. 16A and 16B). These cells represent either a homogenous populations of multipotent progenitors or a heterogeneous population of unipotent progenitors. Culturing R+G− (but not other) progenitors on micro-patterned surfaces resulted in a statistically significant increase in the proportion of cardiac myocytes suggesting that this population's myogenic potential may be modulated by micro-environmental geometric cues.

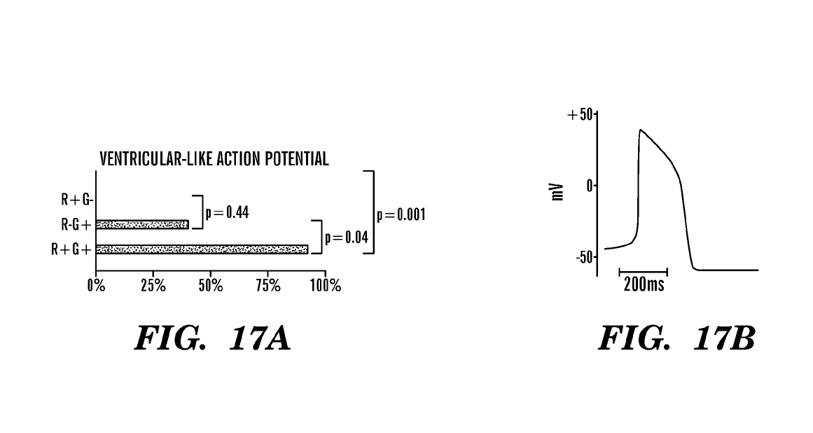

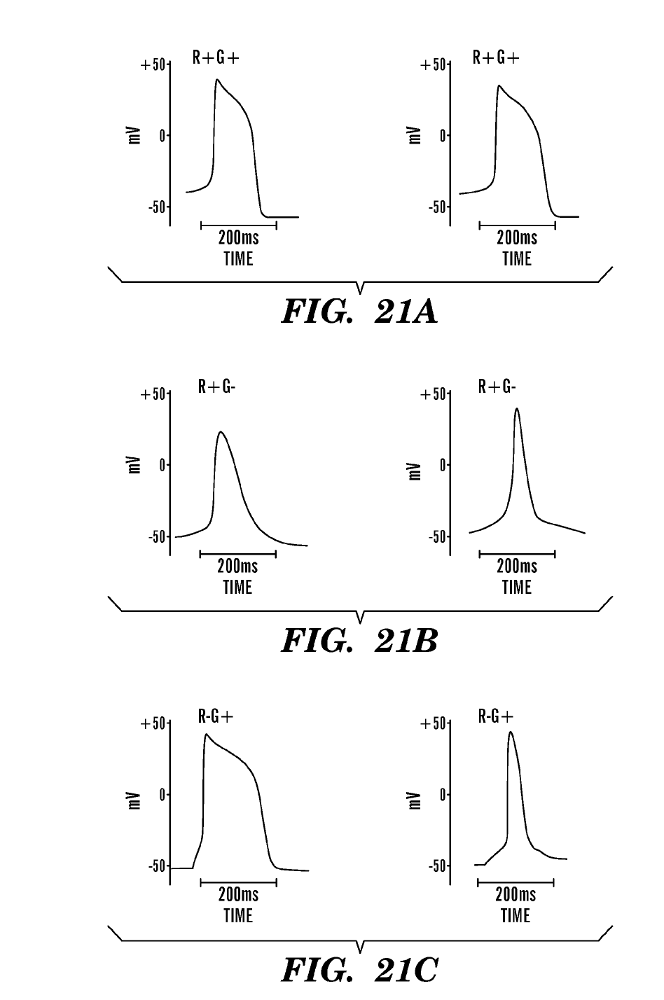

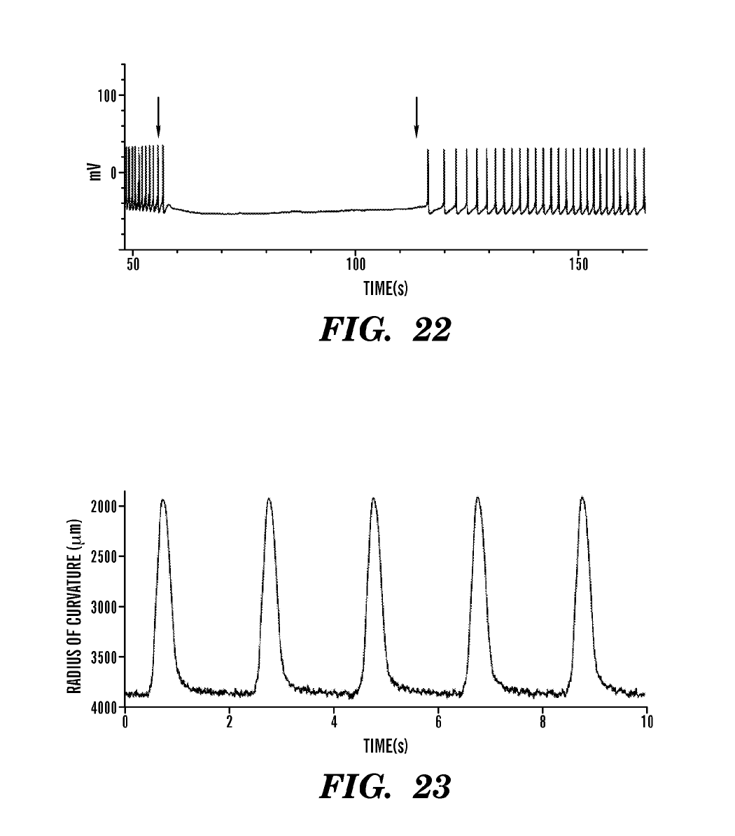

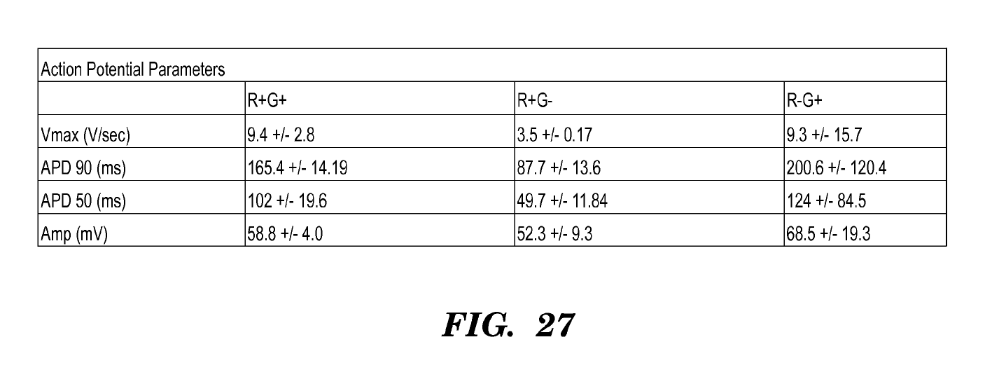

Single cell patch clamp experiments demonstrated that R+G+ progenitors differentiated into ventricular cardiac myocytes with typical four-phase action potential (AP), whereas R+G− and R−G+ progenitors differentiated into more heterogeneous cell types (FIGS. 17A, 17B, 21A-21C, and FIG. 27). Further, R+G+ cardiomyocytes showed sodium channel dependency, consistent with ventricular APs (FIG. 22).

To examine whether ESC-derived ventricular progenitor cells can differentiate into functional, stress generating cardiac muscle, the inventors engineered 2D cardiac tissue anchored on a thin film of PDMS elastomer (as described herein, and in Feinberg et al., Science 317, 1366 (2007), which is incorporated herein in its entirety by reference). EB day 6 dsRed+/eGFP+(R+G+) progenitors were FACS sorted and then allowed to expand and differentiate for an additional 7 days to generate a muscular thin film (MTF). At room temperature the MTF beat spontaneously at a rate of approximately 20 contractions per minute. The MTF could be paced by field stimulation at 0.5 and 1.0 Hz. The stress produced by the cardiac tissue was calculated by measuring the curvature of the MTF, as previously described ((16)). Peak systolic stress generated was measured at −13 kPa at 0.5 Hz pacing (FIG. 9F), comparable to the peak systolic stress generated by thin films engineered from neonatal ventricular cardiomyocytes (16).

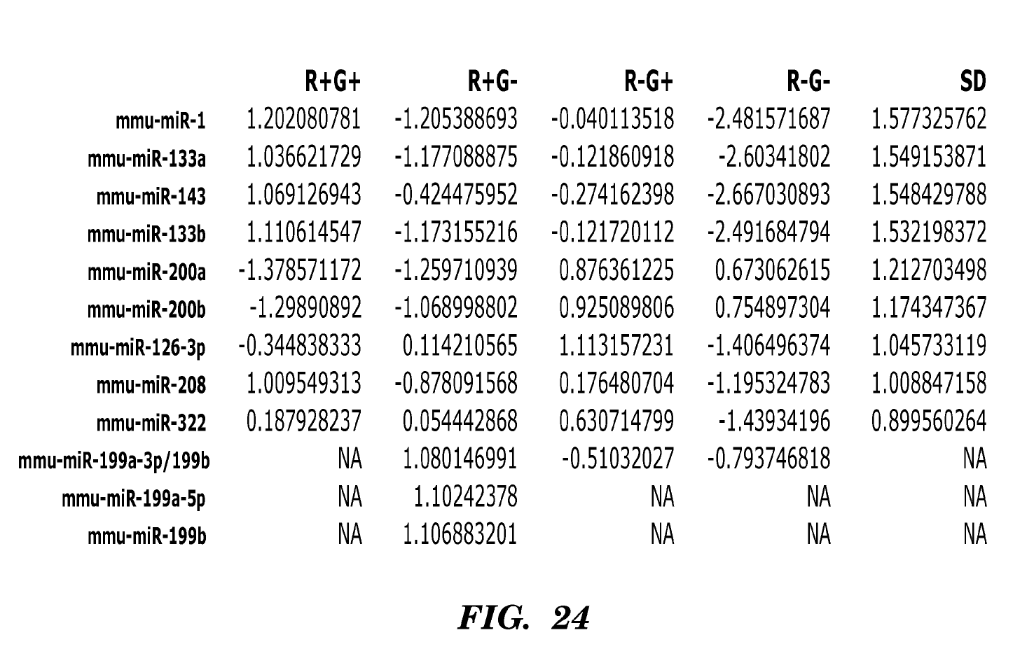

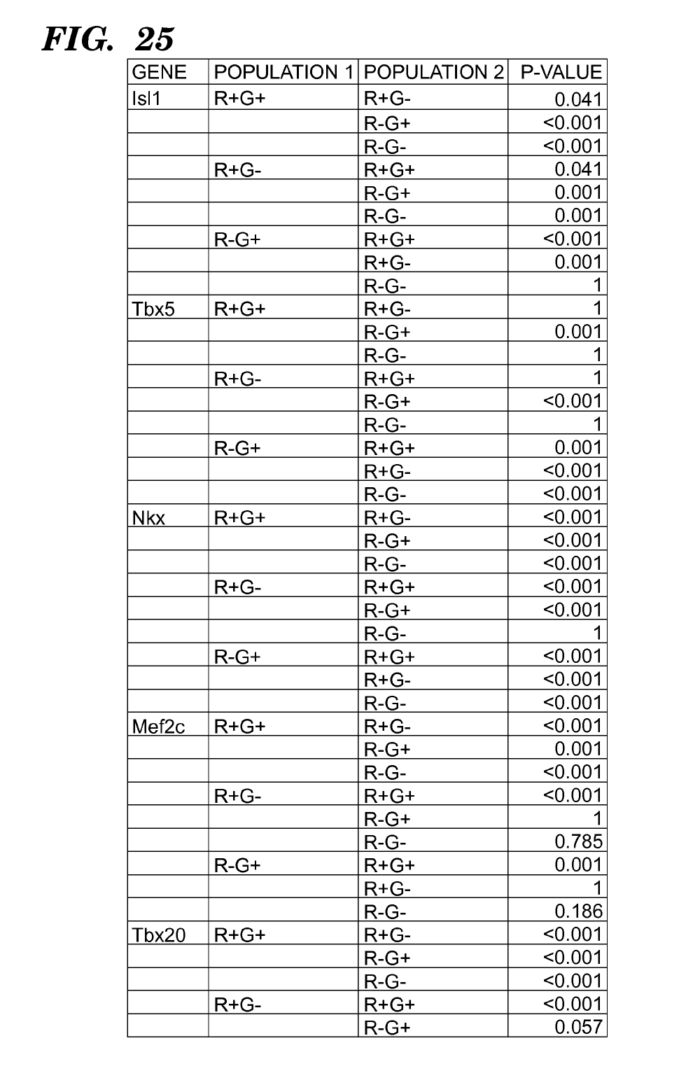

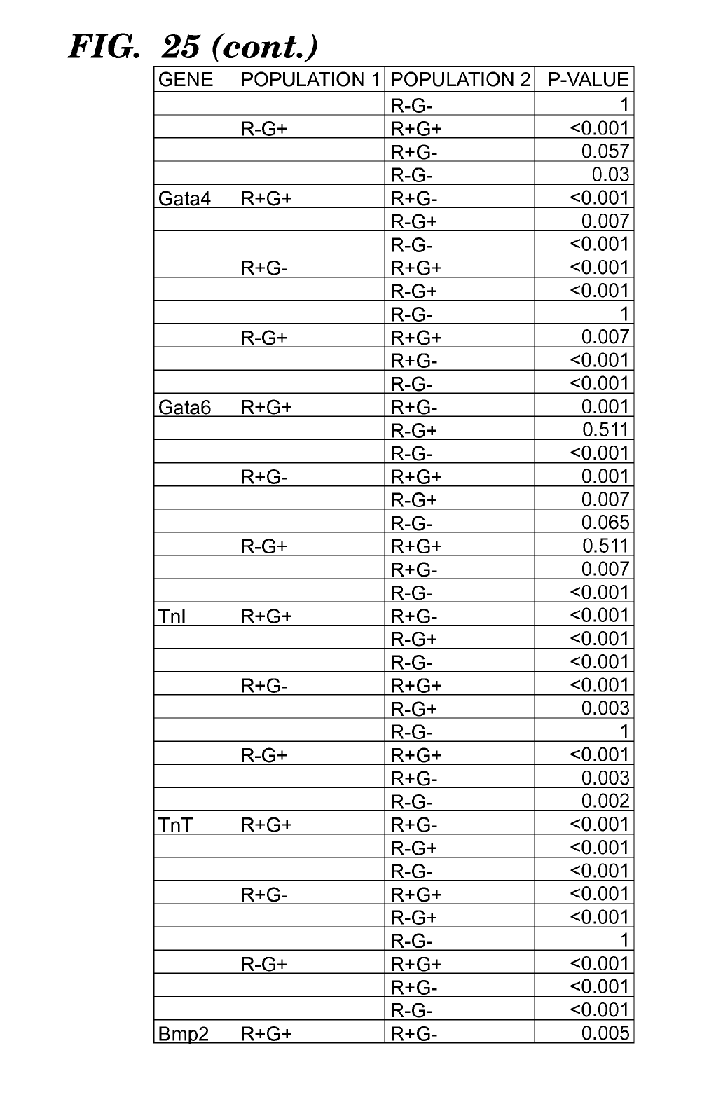

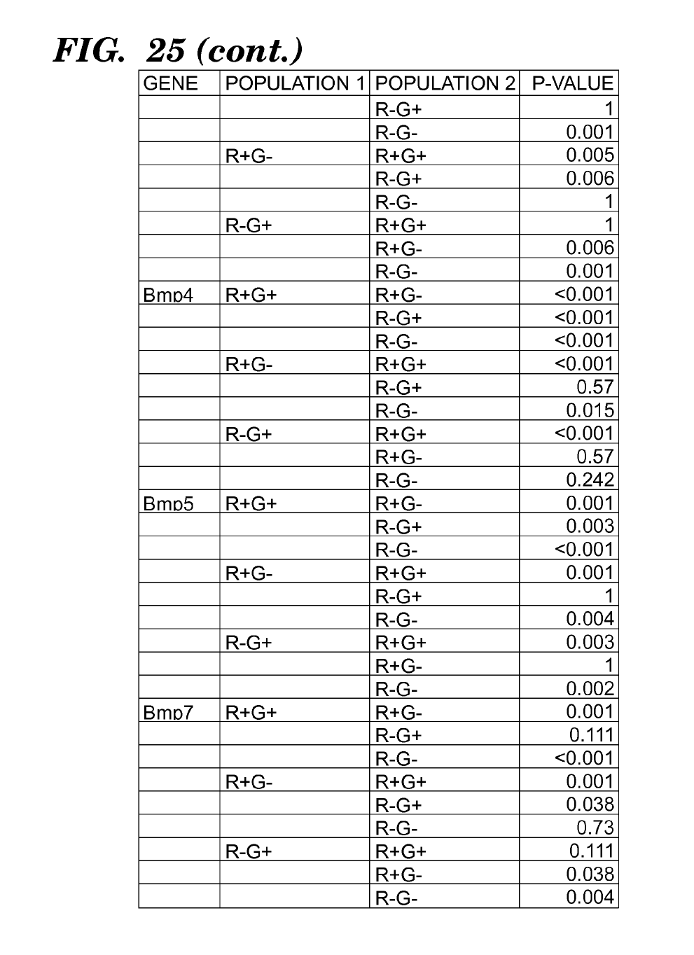

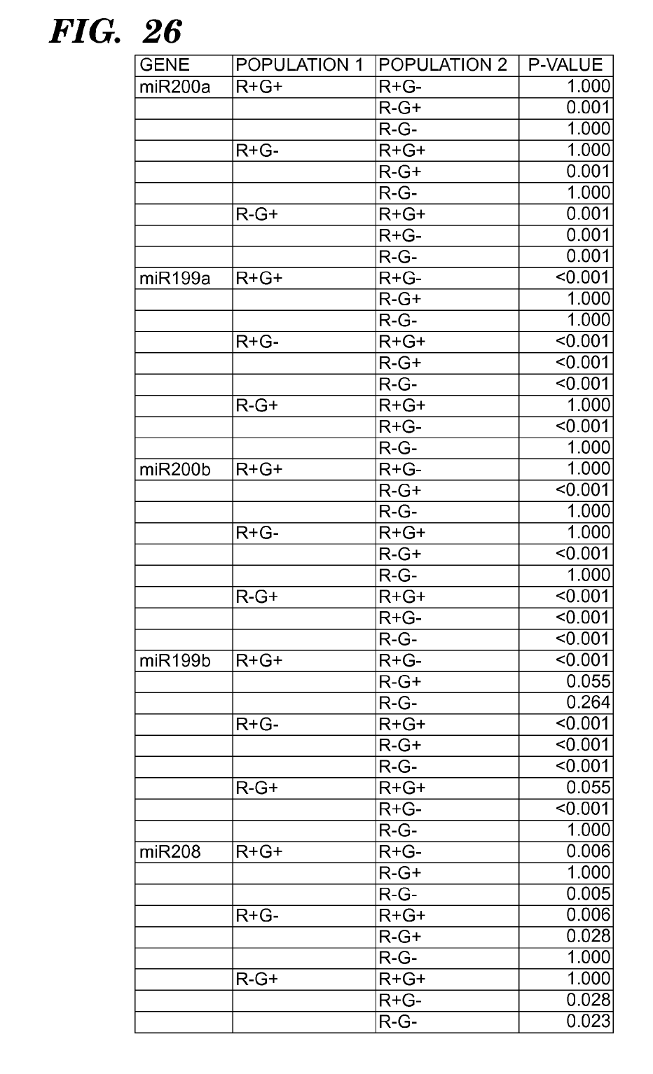

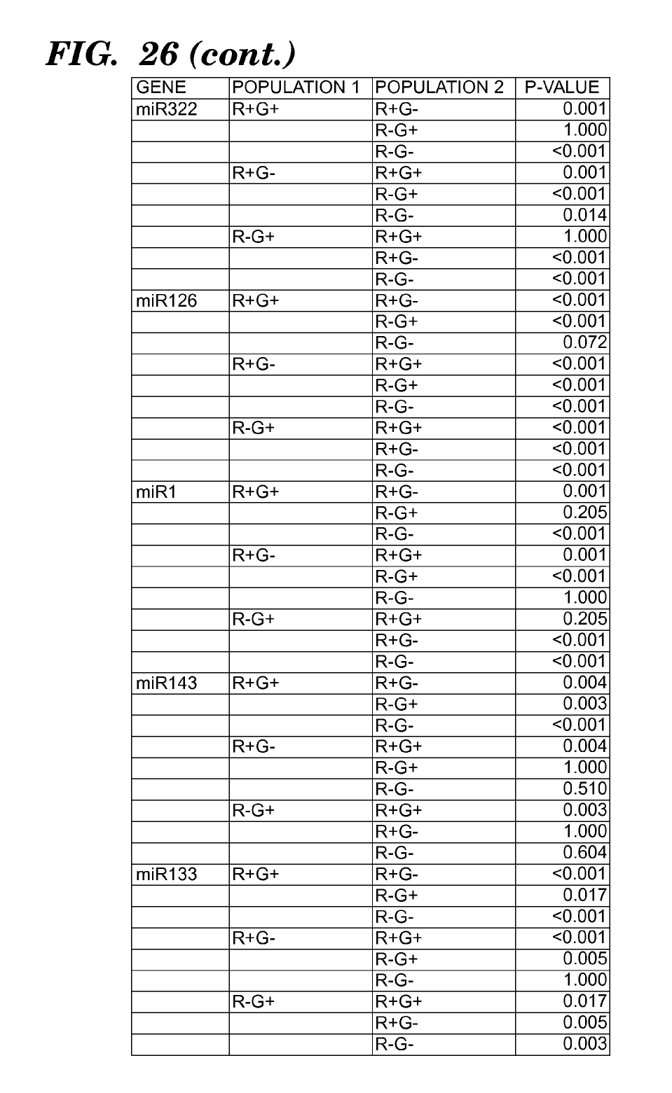

Thus, the inventors have demonstrated the use of the R+G+ progenitors to engineer 2-dimensional (2D) cardiac tissue into a muscular thin film (MTF), using R+G+ progenitors according to the methods as described herein, whereas a Feinberg et al., Science 317, 1366 (2007) engineered MTFs from neonatal rat ventricular cardiomyocytes. The MTF beat spontaneously at a rate of approximately 20 contractions per minute and could be paced by field stimulation at 0.5 and 1.0 Hz. To measure contractility, the MTF was fixed as a cantilever on one end and the contracting cardiomyocytes bent the MTF towards the cell-side during systole (FIG. 9E). During diastole, the elastic polydimethylsiloxane (PDMS) film provided the antagonistic force that returned the MTF back to the relaxed position. The change in radius of curvature is inversely proportional to cardiomyocyte stress generation and was measured at ˜5 kPa for the progenitor-derived cardiac tissue at peak systole (FIG. 23), similar to MTFs engineered from neonatal rat ventricular cardiomyocytes (Feinberg et al., Science 317, 1366 (2007).

The inventors used anisotropic spatial structures such as a thin film of PDMS elastomer, but other structures and other culture surfaces can be used for example, patterned regions of non-adhesive surface chemistry (polyethylene glycol or bovine serum albumin), discrete changes in surface chemistry (protein type, density, activity, etc. . . . ), surface topography, sutures and synthetic or natural fibers or fibrils. These cues can be combined with additional methodologies can be used to enhance muscle generation including electric fields, mechanical stimulation and pharmaceuticals.

Accordingly the inventors herein demonstrate the development and use of an in vivo multicolor reporter system in embryos and corresponding ES cell lines, coupled with FACS analysis of positive and negative signals, to purify distinct subsets of heart field progenitors from the earliest stages of cardiogenesis. Previous studies employing dye labeling, molecular markers, and in vivo lineage tracing have pointed to only two classes of heart progenitors that are localized in the first (FHF) and secondary heart fields (SHF) (19, 30). However, while Islet-1 primarily marks the SHF (6-8, 12, 13), there have been no distinct markers for the first heart field (FHF) lineages that contribute to the left ventricular (LV) chamber. The inventors demonstrate herein, identification and isolation of first heart field (FHF) progenitors which contribute to the ventricular heart chamber, their identification and the direct determination of their relationship to the well-characterized SHF lineages has largely been a source of speculation. The inventors demonstrate distinct transcriptional signatures for the FHF and SHF lineages, including expression of unique subsets of microRNAs, demonstrating that FHF and SHF progenitors have distinct identities. The these unique subsets of microRNAs and expression profiles can be used as FHF markers, and used for identifying FHF progenitors and allow rigorous analysis of the fate of FHF progenitors and their progeny in heard development and disease. In this regard, it was previously suggested that Islet-1 may be transiently expressed even in FHF lineages (31, 32). Contrary to this, the inventors demonstrate herein that isll expression is not definitive marker which can be used to identify cells belonging to the FHF lineage.

Accordingly, the inventors herein have demonstrated and discovered distinct transcriptional signatures for the FHF and SHF lineages that go well beyond the expression of Islet-1, including the expression of unique subsets of microRNAs that have been shown to play critical roles in cardiac muscle cell lineages (33-36). The profiles are sufficiently distinct to indicate that they have non-overlapping identities. The inventors discovery and identification of numerous independent markers for the FHF lineage allows their isolation for any use, such as but not limited to their use to generate tissue engineered myocardium as disclosed herein, as well as in assays to identify agents which affect their function, as well as for the study and analysis of their role in the embryonic, neonatal, and potentially in the adult heart.

A critical step in cardiogenesis is the formation and expansion of ventricular muscle cell lineages (ventricular myocyte lineages), and the subsequent expansion of a sufficient muscle mass to drive and maintain cardiac contractile function. The discovery and purification from embryos and corresponding ES cell lines of committed ventricular muscle cell progenitors (CVPs) from the Islet-1 lineage uncovers a novel mechanistic pathway for the formation and expansion of ventricular muscle mass and for organogeneisis through the expansion and assembly of CVPs into fully functional ventricular muscle tissue. Fully differentiated ventricular muscle cells have an inherently low to negligible proliferative capacity (29), and at the same time, a distinct set of pathways must govern the decision of multipotent islet-1 progenitors to enter the ventricular lineage.

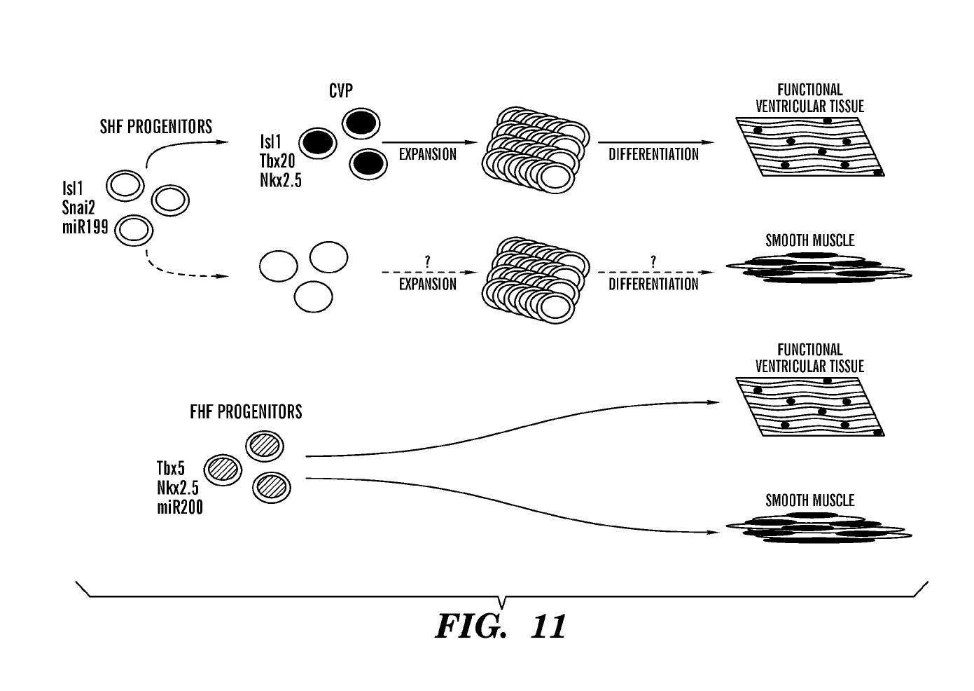

Thus, the inventors have demonstrated that directed differentiation from multipotent islet progenitors to a specific differentiated progeny occurs via the formation of transient committed intermediate progenitor, the CVP intermediate progenitor which is destined to become specific cell types, e.g. ventricular myocytes. The inventors have discovered herein a critical role for committed ventricular progenitor (CVP) cells in development of ventricular myocyte lineages, and demonstrate that the expansion of ventricular cardiac muscle mass occurs via the self renewal and self assembly of CVPs into fully functioning, mature ventricular muscle tissue. The inventors discovery demonstrates a general paradigm for the conversion of multipotent islet progenitors to other differentiated cell types, such as endothelial endocardial, valvular, or conduction system cells (FIG. 11). By creating alternative multicolor reporter systems and FACS analysis, the inventors have demonstrated that it is now become feasible to isolate and directly characterize these progenitors in an analogous fashion. Furthermore, recent work has now identified multiple Isl1 intermediate progenitor populations in human embryonic hearts and human ESC (Bu et al., Nature 460, 113, 2009), indicating that it is possible to isolate self-expanding human ventricular progenitors. Accordingly, using the FHF biomarkers as disclosed herein, CVP cells can be isolated from humans and human embryonic stem cells and from available human embryonic stem cell lines.

Advanced heart failure is a major, unmet clinical need, arising from a loss of viable and/or fully functional cardiac muscle cells (37). Accordingly, designing new approaches to augment the number of functioning human cardiac muscle cells in the failing heart forms a foundation for modern regenerative cardiovascular medicine. Currently, a number of scientific studies and clinical trials have been designed to augment the number of functioning cardiac muscle cells via the transplantation of a diverse group of stem cells and progenitor cells outside of the heart, which might convert to functioning muscle and/or secondarily improve the function to cardiac muscle in the failing heart. To date, while there have been encouraging early suggestions of a small therapeutic benefit, there has not been evidence for the robust regeneration of heart muscle tissue in these clinical studies (38, 39) thereby underscoring the need for new approaches.

A central challenge for cell-based therapy has been the identification of an optimal cell type to drive robust cardiac myogenesis. The ideal heart progenitor cell would be derived from a renewable cell source in sufficient quantities to drive clinically relevant levels of cardiac myogenesis. In addition, it would be critical to direct the differentiation of progenitor cells into functional ventricular myocytes, instead of related lineages such as smooth muscle cells or conduction system muscle cells, that might carry electrophysiological side effects following their implantation. The inventors have demonstrated the ability to generate fully functional ventricular MTF, which can be used for direct chemical screening of novel molecular entities for therapeutic endpoints that can only be measured on intact muscle tissue, including force development and conduction velocity. With recent advances in the generation of induced pluripotent stem cells (iPS) (40-42), the inventors can also isolate CVPs from patients and direct their differentiation into patient and disease specific cardiac progenitors. The combination of tissue engineering technology with stem cell biology, therefore, represents an approach for the development of human models of human disease and a platform for drug discovery and design.

Accordingly, one can generate CVPs for use in the generation of MTF as disclosed herein from iPS sources, and therefore promote the formation of ventricular cardiac myogenesis from cells obtained from a subject which can then be used for direct in vivo cardiac cell transplantation. In addition, the inventors discovery and demonstration of the ability to generate fully functional mature ventricular muscle thin films (MTF) can also be used for the direct chemical screening of novel agents or molecular entities for therapeutic or cardiotoxicity endpoints that can currently only be measured on intact muscle tissue, including tension, force development, work, and conduction velocity.

### REFERENCES

All reference are cited in the specification and the Examples are incorporated in their entirety herein by reference.
1. C. A. Risebro, P. R. Riley, The scientific world journal 6, 1862 (2006).
2. S. Martin-Puig, Z. Wang, K. R. Chien, Cell Stem Cell 2, 320 (2008).
3. S. M. Wu, K. R. Chien, C. Mummery, Cell 132, 537 (2008).
4. D. J. Garry, E. N. Olson, Cell 127, 1101 (Dec. 15, 2006).
5. D. Srivastava, Cell 126, 1037 (Sep. 22, 2006).
6. K. L. Laugwitz et al., Nature 433, 647 (Feb. 10, 2005).
7. A. Moretti et al., Cell 127, 1151 (Dec. 15, 2006).
8. Y. Qyang et al., Cell Stem Cell 1, 165 (2007).
9. S. M. Wu et al., Cell 127, 1137 (Dec. 15, 2006).
10. S. J. Kattman, T. L. Huber, G. M. Keller, Dev Cell 11, 723 (November, 2006).
11. M. Buckingham, S. Meilhac, S. Zaffran, Nat Rev Genet 6, 826 (November, 2005).
12. K. L. Laugwitz, A. Moretti, L. Caron, A. Nakano, K. R. Chien, Development 135, 193 (2008).
13. C. L. Cai et al., Dev Cell 5, 877 (December, 2003).
14. B. Zhou et al., Nature 454, 109 (2008).
15. C. L. Cai et al., Nature 454, 104 (2008).
16. A. W. Feinberg et al., Science 317, 1366 (Sep. 7, 2007).
17. E. Dodou, S. M. Xu, B. L. Black, Mech Dev 120, 1021 (September, 2003).
18. E. Dodou, M. P. Verzi, J. P. Anderson, S. M. Xu, B. L. ack, Development 131, 3931 (August, 2004).
19. K. L. Waldo et al., Development 128, 3179 (August, 2001).
20. C. H. Mjaatvedt et al., Dev Biol 238, 97 (Oct. 1, 2001).
21. R. G. Kelly, N. A. Brown, M. E. Buckingham, Dev Cell 1, 435 (September, 2001).
22. R. G. Kelly, M. E. Buckingham, Trends Genet 18, 210 (April, 2002).
23. C. L. Lien et al., Development 126, 75 (January, 1999).
24. B. G. Bruneau et al., Cell 106, 709 (Sep. 21, 2001).
25. I. P. Moskowitz et al., Development 131, 4107 (August 2004).
26. A. Barrallo-Gimeno, M. A. Nieto, Development 132, 3151 (2005).
27. M. J. Blanco et al., Development 134, 4073 (2007).
28. A. D. Mori et al., Dev Biol 297, 566 (Sep. 15, 2006).
29. G. Brooks, R. A. Poolman, C. J. McGill, J. M. Li, J Mol Cell Cardiol 29, 2261 (August, 1997).
30. K. L. Waldo et al., Dev Biol 281, 78 (May 1, 2005).
31. O. W. Prall et al., Cell 128, 947 (Mar. 9, 2007).
32. Z. B. Ma Q, Pu W T, Dev Biol, (2008).
33. C. Kwon, Z. Han, E. N. Olson, D. Srivastava, Proc Natl Acad Sci USA 102, 18986 (Dec. 27, 2005).
34. E. van Rooij et al., Proc Natl Acad Sci USA 103, 18255 (Nov. 28, 2006).
35. Y. Zhao et al., Cell 129, 303 (Apr. 20, 2007).
36. Y. Zhao, D. Srivastava, Trends Biochem Sci 32, 189 (April, 2007).
37. M. Jessup, S. Brozena, N Engl J Med 348, 2007 (May 15, 2003).
38. P. Menasche, Semin Thorac Cardiovasc Surg 20, 131 (Summer, 2008).
39. J. Pouly et al., J Thorac Cardiovasc Surg 135, 673 (March, 2008).
40. K. Takahashi et al., Cell 131, 861 (Nov. 30, 2007).
41. J. Yu et al., Science 318, 1917 (Dec. 21, 2007).
42. J. Hanna et al., Cell 133, 250 (Apr. 18, 2008).
43. Bentwich, I. (2005). Prediction and validation of microRNAs and their targets. FEBS Lett 579, 5904-5910.
44. Bentwich, I., Avniel, A., Karov, Y., Aharonov, R., Gilad, S., Barad, O., Barzilai, A., Einat, P., Einav, U., Meiri, E., et al. (2005). Identification of hundreds of conserved and nonconserved human microRNAs. Nat Genet 37, 766-770.
45. Bernstein, E., Kim, S. Y., Carmell, M. A., Murchison, E. P., Alcorn, H., Li, M. Z., Mills, A. A., Elledge, S. J., Anderson, K. V., and Hannon, G. J. (2003). Dicer is essential for mouse development. Nat Genet 35, 215-217.
46. Cai, C. L., Liang, X., Shi, Y., Chu, P. H., Pfaff, S. L., Chen, J., and Evans, S. (2003). Isl1 identifies a cardiac progenitor population that proliferates prior to differentiation and contributes a majority of cells to the heart. Dev Cell 5, 877-889.
47. Care, A., Catalucci, D., Felicetti, F., Bonci, D., Addario, A., Gallo, P., Bang, M. L., Segnalini, P., Gu, Y., Dalton, N. D., et al. (2007). MicroRNA-133 controls cardiac hypertrophy. Nat Med 13, 613-618.
48. Carthew, R. W. (2006). Gene regulation by microRNAs. Curr Opin Genet Dev 16, 203-208.
49. Chien, K. R., and Olson, E. N. (2002). Converging pathways and principles in heart development and disease: CV@CSH. Cell 110, 153-162.
50. Cripps, R. M., and Olson, E. N. (2002). Control of cardiac development by an evolutionarily conserved transcriptional network. Dev Biol 246, 14-28.
51. Dodou, E., Verzi, M. P., Anderson, J. P., Xu, S. M., and Black, B. L. (2004). Mef2c is a direct transcriptional target of ISL1 and GATA factors in the anterior heart field during mouse embryonic development. Development 131, 3931-3942.
52. Garry, D. J., and Olson, E. N. (2006). A common progenitor at the heart of development. Cell 127, 1101-1104.
53. Kelly, R. G., and Buckingham, M. E. (2002). The anterior heart-forming field: voyage to the arterial pole of the heart. Trends Genet 18, 210-216.
54. Lee, Y., Ahn, C., Han, J., Choi, H., Kim, J., Yim, J., Lee, J., Provost, P., Radmark, O., Kim, S., and Kim, V. N. (2003). The nuclear RNase III Drosha initiates microRNA processing. Nature 425, 415-419.
55. Lee, Y. S., Nakahara, K., Pham, J. W., Kim, K., He, Z., Sontheimer, E. J., and Carthew, R. W. (2004). Distinct roles for Drosophila Dicer-1 and Dicer-2 in the siRNA/miRNA silencing pathways. Cell 117, 69-81.
56. Lien, C. L., McAnally, J., Richardson, J. A., and Olson, E. N. (2002). Cardiac-specific activity of an Nkx2-5 enhancer requires an evolutionarily conserved Smad binding site. Dev Biol 244, 257-266.
57. Lien, C. L., Wu, C., Mercer, B., Webb, R., Richardson, J. A., and Olson, E. N. (1999). Control of early cardiac-specific transcription of Nkx2-5 by a GATA-dependent enhancer. Development 126, 75-84.
58. Lim, L. P., Lau, N. C., Garrett-Engele, P., Grimson, A., Schelter, J. M., Castle, J., Bartel, D. P., Linsley, P. S., and Johnson, J. M. (2005). Microarray analysis shows that some microRNAs downregulate large numbers of target mRNAs. Nature 433, 769-773.
59. Lin, Q., Schwarz, J., Bucana, C., and Olson, E. N. (1997). Control of mouse cardiac morphogenesis and myogenesis by transcription factor MEF2C. Science 276, 1404-1407.
60. McFadden, D. G., Barbosa, A. C., Richardson, J. A., Schneider, M. D., Srivastava, D., and Olson, E. N. (2005). The Handl and Hand2 transcription factors regulate expansion of the embryonic cardiac ventricles in a gene dosage-dependent manner. Development 132, 189-201.
61. Nakahara, K., and Carthew, R. W. (2004). Expanding roles for miRNAs and siRNAs in cell regulation. Curr Opin Cell Biol 16, 127-133.
62. Olson, E. N. (2006). Gene regulatory networks in the evolution and development of the heart. Science 313, 1922-1927.
63. Sayed, D., Hong, C., Chen, I. Y., Lypowy, J., and Abdellatif, M. (2007). MicroRNAs play an essential role in the development of cardiac hypertrophy. Circ Res 100, 416-424.
64. Srivastava, D., and Olson, E. N. (2000). A genetic blueprint for cardiac development. Nature 407, 221-226.
65. Tam, P. P., and Behringer, R. R. (1997). Mouse gastrulation: the formation of a mammalian body plan. Mech Dev 68, 3-25.
66. van Rooij, E., and Olson, E. N. (2007). MicroRNAs: powerful new regulators of heart disease and provocative therapeutic targets. J Clin Invest 117, 2369-2376.
67. van Rooij, E., Sutherland, L. B., Liu, N., Williams, A. H., McAnally, J., Gerard, R. D., Richardson, J. A., and Olson, E. N. (2006). A signature pattern of stress-responsive microRNAs that can evoke cardiac hypertrophy and heart failure. Proc Natl Acad Sci USA 103, 18255-18260.
68. van Rooij, E., Sutherland, L. B., Qi, X., Richardson, J. A., Hill, J., and Olson, E. N. (2007). Control of stress-dependent cardiac growth and gene expression by a microRNA. Science 316, 575-579.
69. Verzi, M. P., McCulley, D. J., De Val, S., Dodou, E., and Black, B. L. (2005). The right ventricle, outflow tract, and ventricular septum comprise a restricted expression domain within the secondary/anterior heart field. Dev Biol 287, 134-145.
70. Vong, L. H., Ragusa, M. J., and Schwarz, J. J. (2005). Generation of conditional Mef2cloxP/loxP mice for temporal- and tissue-specific analyses. Genesis 43, 43-48.
71. Wu, S. M., Fujiwara, Y., Cibulsky, S. M., Clapham, D. E., Lien, C. L., Schultheiss, T. M., and Orkin, S. H. (2006). Developmental origin of a bipotential myocardial and smooth muscle cell precursor in the mammalian heart. Cell 127, 1137-1150.
72. Xiao, J., Luo, X., Lin, H., Zhang, Y., Lu, Y., Wang, N., Zhang, Y., Yang, B., and Wang, Z. (2007). MicroRNA miR-133 represses HERG K+channel expression contributing to QT prolongation in diabetic hearts. J Biol Chem 282, 12363-12367.
73. Zhao, Y., Ransom, J. F., Li, A., Vedantham, V., von Drehle, M., Muth, A. N., Tsuchihashi, T., McManus, M. T., Schwartz, R. J., and Srivastava, D. (2007). Dysregulation of cardiogenesis, cardiac conduction, and cell cycle in mice lacking miRNA-1-2. Cell 129, 303-317.

1. A composition comprising a substantially pure population of committed ventricular progenitors (CVP), wherein a CVP is positive for the expression of Mef2c+and Nkx2.5+and is capable of differentiating into the right ventricle (RV) and/or outflow tract (OT).

2. The composition of claim 1, further comprising a scaffold.

3. The composition of claim 1, wherein the CVP is positive for the expression of marker genes selected from the group consisting of: Isl1+, Tbx20, GATA4, GATA6, TropininT, Troponin C, BMP7, BMP4 and BMP2.

4. The composition of 1, wherein the CVP is positive for the expression of an miRNA selected from the group consisting of: miRNA-208, miR-143, miR-133a, miR-133b, miR-1, miR-143 and miR-689.

5. The composition of claim 1, wherein the CVP is derived from an ES cell.

6. The composition of claim 1, wherein the CVP is genetically modified.

7. The composition of any of claim 1, wherein the CVP is a mammalian cell.

8. The composition of claim 7, wherein the mammalian cell is a human cell.

9. The composition of claim 1, wherein the CVP is capable of differentiating into a ventricular cardiomyocyte.

10. The composition of claim 1, wherein the composition comprises at least one CVP cell which has a pathological characteristic of a disease or disorder.

11. The composition of claim 2, wherein the scaffold comprises a plurality of freestanding tissue structures, wherein each free standing tissue structure comprises a flexible polymer scaffold imprinted with a predetermined pattern, and the CVPs are arranged in spatially organized manner according to said pattern to yield contractible myocardial tissue.

12. The composition of claim 2, where in the scaffold is a biocompatible substrate, or a biodegradable substrate or a biocompatible and biodegradable substrate.

13. The composition of claim 2, where in the scaffold is a two-dimensional scaffold or a three-dimensional scaffold.

14. The composition of claim 13, wherein the three-dimensional scaffold is a plurality of two dimensional scaffold.

15. The composition of claim 11, wherein the patterned biopolymer structure is a freestanding biopolymer comprising an integral pattern of the biopolymer having repeating features with a dimension of less than 1 mm and without a supporting substrate.

16. The composition of claim 11, wherein the free-standing biopolymer structure comprises an integral pattern of the biopolymer and poly(N-Isopropylacrylamide).

17. The composition of claim 1, wherein the composition forms myocardial tissue which has at least one characteristics which is substantially similar to a characteristic of functional ventricular heart muscle, where a characteristic of functional ventricular heart muscle is selected from the group of: substantially similar contractile force, substantially similar contractile frequency, substantially similar contractile duration and substantially similar contractile stamina.

18. An assay to identify an agent that alters the contractile activity of myocardial tissue, comprising:
a. contacting the myocardial tissue of any of claims 1-18 with at least one agent;
b. measuring the contractile activity of the myocardial tissue in the presence of at least one agent;
c. comparing the contractile activity of the myocardial tissue in the presence of at least one agent with a reference contractile activity of myocardial tissue;
wherein a change in the contractile activity by a statistically significant amount in the presence of the agent as compared to the reference contractile activity identifies an agent that alters the contractile activity.

19. The assay of claim 18, wherein a change in the contractile activity is an increase or decrease in at lease one contractile activity, and wherein a contractile activity is selected from the group consisting of: contractile force, contractile frequency, contractile duration and contractile stamina.

20. The method of claim 18, wherein the reference contractile activity is the contractile activity of the myocardial tissue of claim 17 selected from at least one of: the contractile activity in the absence of an agent, or the contractile activity in the presence of at least one positive control agent, or the contractile activity in the presence of at least one negative control agent.

21. A method of treating a cardiovascular disorder in a subject in need thereof, comprising administering to the subject an effective amount of the composition of any of claims 1 to 17.

22. Use of the composition of any of claims 1 to 17 for the treatment of a cardiovascular disease or disorder in a subject, wherein the composition is administered to the subject by transplantation to the subject in need of treatment.

23. Use of the composition of any of claims 1 to 17 in an assay to identify a cardiotoxic agent.

24. Use of the assay of any of claims 18 to 20 for identifying a cardiotoxic agent.

25. The use of claim 23, wherein an agent which increases or decreases the contractile activity by a statistically significant amount of the composition of any of claims 1-18 is a cardiotoxic agent, and wherein the contractile activity is selected from at least one of the group consisting of: contractile force, contractile frequency, contractile duration and contractile stamina.


SEQUENCE LISTING (32 sequences)

SEQ ID NO: 1 - DNA, 6274 bp, Homo sapiens
aaaactggggggtttctcttcaaagccagctggactggctttattctgcaggaatttttttacctgtcagggtttggacaacaaagccctcagcaggtgctgacgggtacagcttcctggagaagcagaaaggcactggtgccaaagaagagttgcaaactgtgaagtaacttctatgaagagatgaagtaaagaacggaaggcaaatgattgtggcagtaaagaagtgtatgtgcaggaacgaatgcaggaatttgggaactgagctgtgcaagtgctgaagaaggagatttgtttggaggaaacaggaaagagaaagaaaaggaaggaaaaaatacataatttcagggacgagagagagaagaaaaacggggactatggggagaaaaaagattcagattacaaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgcattgaacaagaaagaaaacaaaggctgtgaaagccccgatcccgactcctcttatgcactcaccccacgcactgaagaaaaatacaaaaaaattaatgaagaatttgataatatgatcaagagtcataaaattcctgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacaccaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacatgatcagattattacttactagtttttttttttcttgcagtgtgtgtgtgtgctataccttaatggggaaggggggtcgatatgcattatatgtgccgtgtgtggaaaaaaaaaaagtcaggtactctgttttgtaaaagtacttttaaattgcctcagtgatacagtataaagataaacagaaatgctgagataagcttagcacttgagttgtacaacagaacacttgtacaaaatagattttaaggctaacttcttttcactgttgtgctcctttgcaaaatgtatgttacaatagatagtgtcatgttgcaggttcaacgttatttacatgtaaatagacaaaaggaaacatttgccaaaagcggcagatctttactgaaagagagagcagctgttatgcaacatatagaaaaatgtatagatgcttggacagacccggtaatgggtggccattggtaaatgttaggaacacaccaggtcacctgacatcccaagaatgctcacaaacctgcaggcatatcattggcgtatggcactcattaaaaaggatcagagaccattaaaagaggaccatacctattaaaaaaaaatgtggagttggagggctaacatatttaattaaataaataaataaatctgggtctgcatctcttattaaataaaaatataaaaatatgtacattacattttgcttattttcatataaaaggtaagacagagtttgcaaagcatttgtggctttttgtagtttacttaagccaaaatgtgtttttttccccttgatagcttcgctaatattttaaacagtcctgtaaaaaaccaaaaaggactttttgtatagaaagcactaccctaagccatgaagaactccatgctttgctaaccaagataactgttttctctttgtagaagttttgtttttgaaatgtgtatttctaattatataaaatattaagaatcttttaaaaaaatctgtgaaattaacatgcttgtgtatagctttctaatatatataatattatggtaatagcagaagttttgttatcttaatagcgggaggggggtatatttgtgcagttgcacatttgagtaactattttctttctgttttcttttactctgcttacattttataagtttaaggtcagctgtcaaaaggataacctgtggggttagaacatatcacattgcaacaccctaaattgtttttaatacattagcaatctattgggtcaactgacatccattgtatatactagtttctttcatgctatttttattttgttttttgcatttttatcaaatgcagggcccctttctgatctcaccatttcaccatgcatcttggaattcagtaagtgcatatcctaacttgcccatattctaaatcatctggttggttttcagcctagaatttgatacgctttttagaaatatgcccagaatagaaaagctatgttggggcacatgtcctgcaaatatggccctagaaacaagtgatatggaatttacttggtgaataagttataaattcccacagaagaaaaatgtgaaagactgggtgctagacaagaaggaagcaggtaaagggatagttgctttgtcatccgtttttaattattttaactgacccttgacaatcttgtcagcaatataggactgttgaacaatcccggtgtgtcaggacccccaaatgtcacttctgcataaagcatgtatgtcatctattttttcttcaataaagagatttaatagccatttcaagaaatcccataaagaacctctctatgtccctttttttaatttaaaaaaaatgactcttgtctaatattcgtctataagggattaattttcagaccctttaataagtgagtgccataagaaagtcaatatatattgtttaaaagatatttcagtctaggaaagattttccttctcttggaatgtgaagatctgtcgattcatctccaatcatatgcattgacatacacagcaaagaagatataggcagtaatatcaacactgctatatcatgtgtaggacatttcttatccattttttctcttttacttgcatagttgctatgtgtttctcattgtaaaaggctgccgctgggtggcagaagccaagagaccttattaactaggctatatttttcttaacttgatctgaaatccacaattagaccacaatgcacctttggttgtatccataaaggatgctagcctgccttgtactaatgttttatatattaaaaaaaaaaaaatctatcaaccatttcatatatatcccactactcaaggtatccatggaacatgaaagaataacatttatgcagaggaaaaacaaaaacatccctgaaaatatacacactcatacacacacacgcacaggggaataaaataagaaaaccattttccccaccatagacttgatcccatccttacaacccatccttctaacttgatgtgtataaaatatgcaaacatttcacaaatgttctttgtcatttcaaaatactttagtatatcaatatcagtagataccagtgggtgggaaagggtcattacatgaaaatatgaagaaatagccatattagttttttaacctgcaatttgcctcagcaacaaagaaaaagtgaatttttaatgctgaagataaagtaagctaaagtaccagcagaagccttggctatttatagcagttctgacaatagttttataagaacatgaagagaacagaatcacttgaaaatggatgccagtcatctcttgttcccactactgaattcttataaagtggtggcaagatagggaagggataatctgagaatttttaaaagatgatttaatgagaagaagcacaattttgattttgatgagtcactttctgtaaacaatcttggtctatctttacccttataccttatctgtaatttaccatttattgtatttgcaaagctagtatggtttttaatcacagtaaatcctttgtattccagactttagggcagagccctgagggagtattattttacataacccgtcctagagtaacattttaggcaacattcttcattgcaagtaaaagatccataagtggcattttacacggctgcgagtattgttatatctaatcctattttaaaagatttttggtaatatgaagcttgaatactggtaacagtgatgcaatatacgcaagctgcacaacctgtatattgtatgcattgctgcgtggaggctgtttatttcaacctttttaaaaattgtgttttttagtaaaatggcttattttttcccaaaggtggaatttagcattttgtaatgatgaatataaaaatacctgtcatccccagatcatttaaaagttaactaaagtgagaatgaaaaaacaaaattccaagacactttttaaaagaatgtctgccctcacacacttttatggatttgtttttcttacatacccatcttttaacttagagatagcattttttgccctctttattttgttgtttgtttctccagagagtaaacgctttgtagttctttctttaaaaaacattttttttaaagaagaagaagccacttgaaccctcaataaaggctgttgcctaagcatggcatacttcatctgttctcatttgtgccatctgccgtgatgtcgtcacttttatggcgttaatttcctgccactacagatcttttgaagattgctggaatactggtgtctgttagaatgcttcagactacagatgtaattaaaggcttttcttaatatgttttaaccaaagatgtggagcaatccaagccacatatcttctacatcaaatttttccattttggttattttcataatctggtattgcattttgccttccctgttcatacctcaaattgattcatacctcagtttaattcagagaggtcagttaagtgacggattctgctgtggtttgaatgcagtaccagtgttctcttcgagcaaagtagacctgggtcactgtaggcataggacttggattgcttcagatggtttgctgtatcatttttcttctttttcttttcctggggacttgtttccattaaatgagagtaattaaaatcgcttgtaaatgagggcatacaagcatttgcaacaaatattcaaatagaggctcacagcggcataagctggactttgtcgccactagatgacaagatgttataactaagttaaaccacatctgtgtatctcaagggacttaattcagctgtctgtagtgaataaaagtgggaaattttcaaaagtttctcctgctggaaataaggtataatttgtattttgcagacaattcagtaaagttactggctttcttagtgaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 6458 bp, Homo sapiens
gcgatgtttcaaacgctgtgagctgttctccttttcccattcgtcttctgtcacttccttcctggacgcagttttctggacgagtctggttacttttaatccgaccggccgctgagagccactttctcctcctcctcctcctcctccttctcttcctcctccttcttcctcctcctcctcctcttccgagcggcctcggcgcgcgcgaatgcgcggccccgcgccccccccctcgcgcgcgctcccctcgcgcgcgcgcacacacgcacacatcgtctccagctctctgctcgctctgctcgcagtcacagacacttgagcacacgcgtacacccagacatcttcgggctgctattggattgactttgaaggttctgtgtgggtcgccgtggctgcatgtttgaatcaggtggagaagcacttcaacgctggacgaagtaaagattattgttgttattttttttttctctctctctctctcttaagaaaggaaaatatcccaaggactaatctgatcgggtcttccttcatcaggaacgaatgcaggaatttgggaactgagctgtgcaagtgctgaagaaggagatttgtttggaggaaacaggaaagagaaagaaaaggaaggaaaaaatacataatttcagggacgagagagagaagaaaaacggggactatggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacatgatcagattattacttactagtttttttttttttcttgcagtgtgtgtgtgtgctataccttaatggggaaggggggtcgatatgcattatatgtgccgtgtgtggaaaaaaaaaaagtcaggtactctgttttgtaaaagtacttttaaattgcctcagtgatacagtataaagataaacagaaatgctgagataagcttagcacttgagttgtacaacagaacacttgtacaaaatagattttaaggctaacttcttttcactgttgtgctcctttgcaaaatgtatgttacaatagatagtgtcatgttgcaggttcaacgttatttacatgtaaatagacaaaaggaaacatttgccaaaagcggcagatctttactgaaagagagagcagctgttatgcaacatatagaaaaatgtatagatgcttggacagacccggtaatgggtggccattggtaaatgttaggaacacaccaggtcacctgacatcccaagaatgctcacaaacctgcaggcatatcattggcgtatggcactcattaaaaaggatcagagaccattaaaagaggaccatacctattaaaaaaaaatgtggagttggagggctaacatatttaattaaataaataaataaatctgggtctgcatctcttattaaataaaaatataaaaatatgtacattacattttgcttattttcatataaaaggtaagacagagtttgcaaagcatttgtggctttttgtagtttacttaagccaaaatgtgtttttttccccttgatagcttcgctaatattttaaacagtcctgtaaaaaaccaaaaaggactttttgtatagaaagcactaccctaagccatgaagaactccatgctttgctaaccaagataactgttttctctttgtagaagttttgtttttgaaatgtgtatttctaattatataaaatattaagaatcttttaaaaaaatctgtgaaattaacatgcttgtgtatagctttctaatatatataatattatggtaatagcagaagttttgttatcttaatagcgggaggggggtatatttgtgcagttgcacatttgagtaactattttctttctgttttcttttactctgcttacattttataagtttaaggtcagctgtcaaaaggataacctgtggggttagaacatatcacattgcaacaccctaaattgtttttaatacattagcaatctattgggtcaactgacatccattgtatatactagtttctttcatgctatttttattttgttttttgcatttttatcaaatgcagggcccctttctgatctcaccatttcaccatgcatcttggaattcagtaagtgcatatcctaacttgcccatattctaaatcatctggttggttttcagcctagaatttgatacgctttttagaaatatgcccagaatagaaaagctatgttggggcacatgtcctgcaaatatggccctagaaacaagtgatatggaatttacttggtgaataagttataaattcccacagaagaaaaatgtgaaagactgggtgctagacaagaaggaagcaggtaaagggatagttgctttgtcatccgtttttaattattttaactgacccttgacaatcttgtcagcaatataggactgttgaacaatcccggtgtgtcaggacccccaaatgtcacttctgcataaagcatgtatgtcatctattttttcttcaataaagagatttaatagccatttcaagaaatcccataaagaacctctctatgtccctttttttaatttaaaaaaaatgactcttgtctaatattcgtctataagggattaattttcagaccctttaataagtgagtgccataagaaagtcaatatatattgtttaaaagatatttcagtctaggaaagattttccttctcttggaatgtgaagatctgtcgattcatctccaatcatatgcattgacatacacagcaaagaagatataggcagtaatatcaacactgctatatcatgtgtaggacatttcttatccattttttctcttttacttgcatagttgctatgtgtttctcattgtaaaaggctgccgctgggtggcagaagccaagagaccttattaactaggctatatttttcttaacttgatctgaaatccacaattagaccacaatgcacctttggttgtatccataaaggatgctagcctgccttgtactaatgttttatatattaaaaaaaaaaaatctatcaaccatttcatatatatcccactactcaaggtatccatggaacatgaaagaataacatttatgcagaggaaaaacaaaaacatccctgaaaatatacacactcatacacacacacgcacaggggaataaaataagaaaatcattttcctcaccatagacttgatcccatccttacaacccatccttctaacttgatgtgtataaaatatgcaaacatttcacaaatgttctttgtcatttcaaaatactttagtatatcaatatcagtagataccagtgggtgggaaagggtcattacatgaaaatatgaagaaatagccatattagttttttaacctgcaatttgcctcagcaacaaagaaaaagtgaatttttaatgctgaagataaagtaagctaaagtaccagcagaagccttggctatttatagcagttctgacaatagttttataagaacatgaagagaacagaatcacttgaaaatggatgccagtcatctcttgttcccactactgaattcttataaagtggtggcaagatagggaagggataatctgagaatttttaaaagatgatttaatgagaagaagcacaattttgattttgatgagtcactttctgtaaacaatcttggtctatctttacccttataccttatctgtaatttaccatttattgtatttgcaaagctagtatggtttttaatcacagtaaatcctttgtattccagactttagggcagagccctgagggagtattattttacataacccgtcctagagtaacattttaggcaacattcttcattgcaagtaaaagatccataagtggcattttacacggctgcgagtattgttatatctaatcctattttaaaagatttttggtaatatgaagcttgaatactggtaacagtgatgcaatatacgcaagctgcacaacctgtatattgtatgcattgctgcgtggaggctgtttatttcaacctttttaaaaattgtgttttttagtaaaatggcttattttttcccaaaggtggaatttagcattttgtaatgatgaatataaaaatacctgtcatccccagatcatttaaaagttaactaaagtgagaatgaaaaaacaaaattccaagacactttttaaaagaatgtctgccctcacacacttttatggatttgtttttcttacatacccatcttttaacttagagatagcattttttgccctctttattttgttgtttgtttctccagagagtaaacgctttgtagttctttctttaaaaaacattttttttaaagaagaagaagccacttgaaccctcaataaaggctgttgcctaagcatggcatacttcatctgttctcatttgtgccatctgccgtgatgtcgtcacttttatggcgttaatttcctgccactacagatcttttgaagattgctggaatactggtgtctgttagaatgcttcagactacagatgtaattaaaggcttttcttaatatgttttaaccaaagatgtggagcaatccaagccacatatcttctacatcaaatttttccattttggttattttcataatctggtattgcattttgccttccctgttcatacctcaaattgattcatacctcagtttaattcagagaggtcagttaagtgacggattctgttgtggtttgaatgcagtaccagtgttctcttcgagcaaagtagacctgggtcactgtaggcataggacttggattgcttcagatggtttgctgtatcatttttcttctttttcttttcctggggacttgtttccattaaatgagagtaattaaaatcgcttgtaaatgagggcatacaagcatttgcaacaaatattcaaatagaggctcacagcggcataagctggactttgtcgccactagatgacaagatgttataactaagttaaaccacatctgtgtatctcaagggacttaattcagctgtctgtagtgaataaaagtgggaaattttcaaaagtttctcctgctggaaataaggtataatttgtattttgcagacaattcagtaaagttactggctttcttagtga

SEQ ID NO: 3 - DNA, 975 bp, Homo sapiens
atgttccccagccctgctctcacgcccacgcccttctcagtcaaagacatcctaaacctggagcagcagcagcgcagcctggctgccgccggagagctctctgcccgcctggaggcgaccctggcgccctcctcctgcatgctggccgccttcaagccagaggcctacgctgggcccgaggcggctgcgccgggcctcccagagctgcgcgcagagctgggccgcgcgccttcaccggccaagtgtgcgtctgcctttcccgccgcccccgccttctatccacgtgcctacagcgaccccgacccagccaaggaccctagagccgaaaagaaagagctgtgcgcgctgcagaaggcggtggagctggagaagacagaggcggacaacgcggagcggccccgggcgcgacggcggaggaagccgcgcgtgctcttctcgcaggcgcaggtctatgaactggagcggcgcttcaagcaacagcggtacctgtcggcccccgaacgcgaccagctggccagcgtgctgaaactcacgtccacgcaggtcaagatctggttccagaaccggcgctacaagtgcaagcggcagcggcaggaccagactctggagctggtggggctgcccccgccgccgccgccgcctgcccgcaggatcgcggtgccagtgctggtgcgcgatggcaagccatgcctaggggactcggcgccctacgcgcctgcctacggcgtgggcctcaatccctacggttataacgcctaccccgcctatccgggttacggcggcgcggcctgcagccctggctacagctgcactgccgcttaccccgccgggccttccccagcgcagccggccactgccgccgccaacaacaacttcgtgaacttcggcgtcggggacttgaatgcggttcagagccccgggattccgcagagcaactcgggagtgtccacgctgcatggtatccgagcctggtag

SEQ ID NO: 4 - DNA, 1585 bp, Homo sapiens
gcctggtcccgcctctcctgccccttgtgctcagcgctacctgctgcccggacacatccagagctggccgacgggtgcgcgggcgggcggcggcaccatgcagggaagctgccaggggccgtgggcagcgccgctttctgccgcccacctggcgctgtgagactggcgctgccaccatgttccccagccctgctctcacgcccacgcccttctcagtcaaagacatcctaaacctggaacagcagcagcgcagcctggctgccgccggagagctctctgcccgcctggaggcgaccctggcgccctcctcctgcatgctggccgccttcaagccagaggcctacgctgggcccgaggcggctgcgccgggcctcccagagctgcgcgcagagctgggccgcgcgccttcaccggccaagtgtgcgtctgcctttcccgccgcccccgccttctatccacgtgcctacagcgaccccgacccagccaaggaccctagagccgaaaagaaagagctgtgcgcgctgcagaaggcggtggagctggagaagacagaggcggacaacgcggagcggccccgggcgcgacggcggaggaagccgcgcgtgctcttctcgcaggcgcaggtctatgagctggagcggcgcttcaagcagcagcggtacctgtcggcccccgaacgcgaccagctggccagcgtgctgaaactcacgtccacgcaggtcaagatctggttccagaaccggcgctacaagtgcaagcggcagcggcaggaccagactctggagctggtggggctgcccccgccgccgccgccgcctgcccgcaggatcgcggtgccagtgctggtgcgcgatggcaagccatgcctaggggactcggcgccctacgcgcctgcctacggcgtgggcctcaatccctacggttataacgcctaccccgcctatccgggttacggcggcgcggcctgcagccctggctacagctgcactgccgcttaccccgccgggccttccccagcgcagccggccactgccgccgccaacaacaacttcgtgaacttcggcgtcggggacttgaatgcggttcagagccccgggattccgcagagcaactcgggagtgtccacgctgcatggtatccgagcctggtagggaagggacccgcgtggcgcgaccctgaccgatcccacctcaacagctccctgactctcggggggagaaggggctcccaacatgaccctgagtcccctggattttgcattcactcctgcggagacctaggaactttttctgtcccacgcgcgtttgttcttgcgcacgggagagtttgtggcggcgattatgcagcgtgcaatgagtgatcctgcagcctggtgtcttagctgtccccccaggagtgccctccgagagtccatgggcacccccggttggaactgggactgagctcgggcacgcagggcctgagatctggccgcccattccgcgagccagggccgggcgcccgggcctttgctatctcgccgtcgcccgcccacgcacccacccgtatttatgtttttacctattgctgtaagaaatgacgatcc

SEQ ID NO: 5 - DNA, 21 bp, Artificial Sequence
  Description of Artificial Sequence Synthetic primer
gcttccgtccctttcatttct

SEQ ID NO: 6 - DNA, 23 bp, Artificial Sequence
  Description of Artificial Sequence Synthetic primer
agcctccatttttggtaaggttt

SEQ ID NO: 7 - DNA, 21 bp, Artificial Sequence
  Description of Artificial Sequence Synthetic primer
ttcctggtaaccgaatgctga

SEQ ID NO: 8 - DNA, 21 bp, Artificial Sequence
  Description of Artificial Sequence Synthetic primer
cctgaatctcggcgacttttt

SEQ ID NO: 9 - DNA, 22 bp, Artificial Sequence
  Description of Artificial Sequence Synthetic primer
ttacttaggggtattgtgggct

SEQ ID NO: 10 - DNA, 21 bp, Artificial Sequence
  Description of Artificial Sequence Synthetic primer
ccgtctctcatggttccgtag

SEQ ID NO: 11 - DNA, 20 bp, Artificial Sequence
  Description of Artificial Sequence Synthetic primer
acggacagggcttctcctac

SEQ ID NO: 12 - DNA, 21 bp, Artificial Sequence
  Description of Artificial Sequence Synthetic primer
atggtggtatcgagggtggaa

SEQ ID NO: 13 - DNA, 21 bp, Artificial Sequence
  Description of Artificial Sequence Synthetic primer
tctgccaactaccgagcctat

SEQ ID NO: 14 - DNA, 21 bp, Artificial Sequence
  Description of Artificial Sequence Synthetic primer
ctcttctgcctctcgttccat

SEQ ID NO: 15 - DNA, 32 bp, Artificial Sequence
  Description of Artificial Sequence Synthetic primer
ttaaggcgcgccgttcttgtctgcctcgtgct

SEQ ID NO: 16 - DNA, 32 bp, Artificial Sequence
  Description of Artificial Sequence Synthetic primer
gcgcttaattaaccgagcaggaatttgaagag

SEQ ID NO: 17 - DNA, 20 bp, Artificial Sequence
  Description of Artificial Sequence Synthetic primer
ttgctccggtaacagcagtg

SEQ ID NO: 18 - DNA, 21 bp, Artificial Sequence
  Description of Artificial Sequence Synthetic primer
gtggtcgcttgtgtagaagga

SEQ ID NO: 19 - DNA, 23 bp, Artificial Sequence
  Description of Artificial Sequence Synthetic primer
atgatggtggtttacaggctaac

SEQ ID NO: 20 - DNA, 21 bp, Artificial Sequence
  Description of Artificial Sequence Synthetic primer
tcgatgctacttcactgccag

SEQ ID NO: 21 - DNA, 33 bp, Artificial Sequence
  Description of Artificial Sequence Synthetic primer
ttaaggcgcgccgagagaagaaacacggggact

SEQ ID NO: 22 - DNA, 31 bp, Artificial Sequence
  Description of Artificial Sequence Synthetic primer
gcgcttaattaaaaaggggtggatgttgagc

SEQ ID NO: 23 - DNA, 32 bp, Artificial Sequence
  Description of Artificial Sequence Synthetic primer
ttaaggcgcgccagcaccactctctgctaccc

SEQ ID NO: 24 - DNA, 32 bp, Artificial Sequence
  Description of Artificial Sequence Synthetic primer
gcgcttaattaatcatcgcccttctcctaaag

SEQ ID NO: 25 - DNA, 23 bp, Artificial Sequence
  Description of Artificial Sequence Synthetic primer
tggtcaagaaacatttcaacgcc

SEQ ID NO: 26 - DNA, 23 bp, Artificial Sequence
  Description of Artificial Sequence Synthetic primer
ggtgaggatctctggttttggta

SEQ ID NO: 27 - DNA, 22 bp, Artificial Sequence
  Description of Artificial Sequence Synthetic primer
aaacccctggaacaatttgtgg

SEQ ID NO: 28 - DNA, 21 bp, Artificial Sequence
  Description of Artificial Sequence Synthetic primer
catctcttcgctggggatgat

SEQ ID NO: 29 - DNA, 32 bp, Artificial Sequence
  Description of Artificial Sequence Synthetic primer
ttaaggcgcgcccccctgtacagagcgagaat

SEQ ID NO: 30 - DNA, 32 bp, Artificial Sequence
  Description of Artificial Sequence Synthetic primer
gcgcttaattaatctctctctctccccacacc

SEQ ID NO: 31 - DNA, 21 bp, Artificial Sequence
  Description of Artificial Sequence Synthetic primer
cagaggaggccaacgtagaag

SEQ ID NO: 32 - DNA, 20 bp, Artificial Sequence
  Description of Artificial Sequence Synthetic primer
ctccatcggggatcttgggt